SEQUENCE LISTING (286 sequences)

SEQ ID NO: 1 - protein, 574 aa, unidentified
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 2 - protein, 60 aa, unidentified
SKVLHLEGEVNKIKSALLSTNKAVVSLSNGVSVLTSKVLDLKNYIDKQLLPIVNKQSCSI

SEQ ID NO: 3 - protein, 49 aa, unidentified
NFYDPLVFPSDEFDASISQVNEKINQSLAFIRKSDELLHNVNAGKSTTN

SEQ ID NO: 4 - protein, 35 aa, synthetic construct
  SITE 3: note = (R)-alpha-(7'-octenyl)alanine
  SITE 10: note = (S)-alpha-(4'-pentenyl)alanine
FDXSISQVNXKINQSLAFIRKSDELLHNVNAGKST

SEQ ID NO: 5 - protein, 30 aa, synthetic construct
  SITE 3: note = (R)-alpha-(7'-octenyl)alanine
  SITE 10: note = (S)-alpha-(4'-pentenyl)alanine
  SITE 30: note = Residue side chain modified at the epsilon carbon with 4x polyethylene glycol attached to thiocholesterol
FDXSISQVNXKINQSLAFIRKSDELLHNVK

SEQ ID NO: 6 - protein, 30 aa, synthetic construct
  SITE 1: note = (R)-alpha-(7'-octenyl)alanine
  SITE 8: note = (S)-alpha-(4'-pentenyl)alanine
  SITE 30: note = Residue side chain modified at the epsilon carbon with 4x polyethylene glycol attached to thiocholesterol
XDASISQXNEKINQSLAFIRKSDELLHNVK

SEQ ID NO: 7 - protein, 30 aa, synthetic construct
  SITE 2: note = (R)-alpha-(7'-octenyl)alanine
  SITE 9: note = (S)-alpha-(4'-pentenyl)alanine
  SITE 30: note = Residue side chain modified at the epsilon carbon with 4x polyethylene glycol attached to thiocholesterol
FXASISQVXEKINQSLAFIRKSDELLHNVK

SEQ ID NO: 8 - protein, 30 aa, synthetic construct
  SITE 4: note = (R)-alpha-(7'-octenyl)alanine
  SITE 11: note = (S)-alpha-(4'-pentenyl)alanine
  SITE 30: note = Residue side chain modified at the epsilon carbon with 4x polyethylene glycol attached to thiocholesterol
FDAXISQVNEXINQSLAFIRKSDELLHNVK

SEQ ID NO: 9 - protein, 30 aa, synthetic construct
  SITE 5: note = (R)-alpha-(7'-octenyl)alanine
  SITE 12: note = (S)-alpha-(4'-pentenyl)alanine
  SITE 30: note = Residue side chain modified at the epsilon carbon with 4x polyethylene glycol attached to thiocholesterol
FDASXSQVNEKXNQSLAFIRKSDELLHNVK

SEQ ID NO: 10 - protein, 30 aa, synthetic construct
  SITE 6: note = (R)-alpha-(7'-octenyl)alanine
  SITE 13: note = (S)-alpha-(4'-pentenyl)alanine
  SITE 30: note = Residue side chain modified at the epsilon carbon with 4x polyethylene glycol attached to thiocholesterol
FDASIXQVNEKIXQSLAFIRKSDELLHNVK

SEQ ID NO: 11 - protein, 30 aa, synthetic construct
  SITE 7: note = (R)-alpha-(7'-octenyl)alanine
  SITE 14: note = (S)-alpha-(4'-pentenyl)alanine
  SITE 30: note = Residue side chain modified at the epsilon carbon with 4x polyethylene glycol attached to thiocholesterol
FDASISXVNEKINXSLAFIRKSDELLHNVK

SEQ ID NO: 12 - protein, 30 aa, synthetic construct
  SITE 8: note = (R)-alpha-(7'-octenyl)alanine
  SITE 15: note = (S)-alpha-(4'-pentenyl)alanine
  SITE 30: note = Residue side chain modified at the epsilon carbon with 4x polyethylene glycol attached to thiocholesterol
FDASISQXNEKINQXLAFIRKSDELLHNVK

SEQ ID NO: 13 - protein, 30 aa, synthetic construct
  SITE 9: note = (R)-alpha-(7'-octenyl)alanine
  SITE 16: note = (S)-alpha-(4'-pentenyl)alanine
  SITE 30: note = Residue side chain modified at the epsilon carbon with 4x polyethylene glycol attached to thiocholesterol
FDASISQVXEKINQSXAFIRKSDELLHNVK

SEQ ID NO: 14 - protein, 30 aa, synthetic construct
  SITE 10: note = (R)-alpha-(7'-octenyl)alanine
  SITE 17: note = (S)-alpha-(4'-pentenyl)alanine
  SITE 30: note = Residue side chain modified at the epsilon carbon with 4x polyethylene glycol attached to thiocholesterol
FDASISQVNXKINQSLXFIRKSDELLHNVK

SEQ ID NO: 15 - protein, 30 aa, synthetic construct
  SITE 11: note = (R)-alpha-(7'-octenyl)alanine
  SITE 18: note = (S)-alpha-(4'-pentenyl)alanine
  SITE 30: note = Residue side chain modified at the epsilon carbon with 4x polyethylene glycol attached to thiocholesterol
FDASISQVNEXINQSLAXIRKSDELLHNVK

SEQ ID NO: 16 - protein, 30 aa, synthetic construct
  SITE 12: note = (R)-alpha-(7'-octenyl)alanine
  SITE 19: note = (S)-alpha-(4'-pentenyl)alanine
  SITE 30: note = Residue side chain modified at the epsilon carbon with 4x polyethylene glycol attached to thiocholesterol
FDASISQVNEKXNQSLAFXRKSDELLHNVK

SEQ ID NO: 17 - protein, 30 aa, synthetic construct
  SITE 13: note = (R)-alpha-(7'-octenyl)alanine
  SITE 20: note = (S)-alpha-(4'-pentenyl)alanine
  SITE 30: note = Residue side chain modified at the epsilon carbon with 4x polyethylene glycol attached to thiocholesterol
FDASISQVNEKIXQSLAFIXKSDELLHNVK

SEQ ID NO: 18 - protein, 30 aa, synthetic construct
  SITE 14: note = (R)-alpha-(7'-octenyl)alanine
  SITE 21: note = (S)-alpha-(4'-pentenyl)alanine
  SITE 30: note = Residue side chain modified at the epsilon carbon with 4x polyethylene glycol attached to thiocholesterol
FDASISQVNEKINXSLAFIRXSDELLHNVK

SEQ ID NO: 19 - protein, 30 aa, synthetic construct
  SITE 15: note = (R)-alpha-(7'-octenyl)alanine
  SITE 22: note = (S)-alpha-(4'-pentenyl)alanine
  SITE 30: note = Residue side chain modified at the epsilon carbon with 4x polyethylene glycol attached to thiocholesterol
FDASISQVNEKINQXLAFIRKXDELLHNVK

SEQ ID NO: 20 - protein, 30 aa, synthetic construct
  SITE 16: note = (R)-alpha-(7'-octenyl)alanine
  SITE 23: note = (S)-alpha-(4'-pentenyl)alanine
  SITE 30: note = Residue side chain modified at the epsilon carbon with 4x polyethylene glycol attached to thiocholesterol
FDASISQVNEKINQSXAFIRKSXELLHNVK

SEQ ID NO: 21 - protein, 30 aa, synthetic construct
  SITE 17: note = (R)-alpha-(7'-octenyl)alanine
  SITE 24: note = (S)-alpha-(4'-pentenyl)alanine
  SITE 30: note = Residue side chain modified at the epsilon carbon with 4x polyethylene glycol attached to thiocholesterol
FDASISQVNEKINQSLXFIRKSDXLLHNVK

SEQ ID NO: 22 - protein, 30 aa, synthetic construct
  SITE 18: note = (R)-alpha-(7'-octenyl)alanine
  SITE 25: note = (S)-alpha-(4'-pentenyl)alanine
  SITE 30: note = Residue side chain modified at the epsilon carbon with 4x polyethylene glycol attached to thiocholesterol
FDASISQVNEKINQSLAXIRKSDEXLHNVK

SEQ ID NO: 23 - protein, 30 aa, synthetic construct
  SITE 19: note = (R)-alpha-(7'-octenyl)alanine
  SITE 26: note = (S)-alpha-(4'-pentenyl)alanine
  SITE 30: note = Residue side chain modified at the epsilon carbon with 4x polyethylene glycol attached to thiocholesterol
FDASISQVNEKINQSLAFXRKSDELXHNVK

SEQ ID NO: 24 - protein, 30 aa, synthetic construct
  SITE 20: note = (R)-alpha-(7'-octenyl)alanine
  SITE 27: note = (S)-alpha-(4'-pentenyl)alanine
  SITE 30: note = Residue side chain modified at the epsilon carbon with 4x polyethylene glycol attached to thiocholesterol
FDASISQVNEKINQSLAFIXKSDELLXNVK

SEQ ID NO: 25 - protein, 30 aa, synthetic construct
  SITE 21: note = (R)-alpha-(7'-octenyl)alanine
  SITE 28: note = (S)-alpha-(4'-pentenyl)alanine
  SITE 30: note = Residue side chain modified at the epsilon carbon with 4x polyethylene glycol attached to thiocholesterol
FDASISQVNEKINQSLAFIRXSDELLHXVK

SEQ ID NO: 26 - protein, 30 aa, synthetic construct
  SITE 22: note = (R)-alpha-(7'-octenyl)alanine
  SITE 29: note = (S)-alpha-(4'-pentenyl)alanine
  SITE 30: note = Residue side chain modified at the epsilon carbon with 4x polyethylene glycol attached to thiocholesterol
FDASISQVNEKINQSLAFIRKXDELLHNXK

SEQ ID NO: 27 - protein, 29 aa, synthetic construct
  VARIANT 1: note = Alpha, alpha-disubstituted non-natural amino acid with an olefinic side chain
  VARIANT 8: note = Alpha, alpha-disubstituted non-natural amino acid with an olefinic side chain
  SITE 1..8: note = Residues are crosslinked
XDASISQXNEKINQSLAFIRKSDELLHNV

SEQ ID NO: 28 - protein, 29 aa, synthetic construct
  VARIANT 2: note = Alpha, alpha-disubstituted non-natural amino acid with an olefinic side chain
  VARIANT 9: note = Alpha, alpha-disubstituted non-natural amino acid with an olefinic side chain
  SITE 2..9: note = Residues are crosslinked
FXASISQVXEKINQSLAFIRKSDELLHNV

SEQ ID NO: 29 - protein, 29 aa, synthetic construct
  VARIANT 3: note = Alpha, alpha-disubstituted non-natural amino acid with an olefinic side chain
  VARIANT 10: note = Alpha, alpha-disubstituted non-natural amino acid with an olefinic side chain
  SITE 3..10: note = Residues are crosslinked
FDXSISQVNXKINQSLAFIRKSDELLHNV

SEQ ID NO: 30 - protein, 29 aa, synthetic construct
  VARIANT 4: note = Alpha, alpha-disubstituted non-natural amino acid with an olefinic side chain
  VARIANT 11: note = Alpha, alpha-disubstituted non-natural amino acid with an olefinic side chain
  SITE 4..11: note = Residues are crosslinked
FDAXISQVNEXINQSLAFIRKSDELLHNV

SEQ ID NO: 31 - protein, 29 aa, synthetic construct
  VARIANT 5: note = Alpha, alpha-disubstituted non-natural amino acid with an olefinic side chain
  VARIANT 12: note = Alpha, alpha-disubstituted non-natural amino acid with an olefinic side chain
  SITE 5..12: note = Residues are crosslinked
FDASXSQVNEKXNQSLAFIRKSDELLHNV

SEQ ID NO: 32 - protein, 29 aa, synthetic construct
  VARIANT 6: note = Alpha, alpha-disubstituted non-natural amino acid with an olefinic side chain
  VARIANT 13: note = Alpha, alpha-disubstituted non-natural amino acid with an olefinic side chain
  SITE 6..13: note = Residues are crosslinked
FDASIXQVNEKIXQSLAFIRKSDELLHNV

SEQ ID NO: 33 - protein, 29 aa, synthetic construct
  VARIANT 7: note = Alpha, alpha-disubstituted non-natural amino acid with an olefinic side chain
  VARIANT 14: note = Alpha, alpha-disubstituted non-natural amino acid with an olefinic side chain
  SITE 7..14: note = Residues are crosslinked
FDASISXVNEKINXSLAFIRKSDELLHNV

SEQ ID NO: 34 - protein, 29 aa, synthetic construct
  VARIANT 8: note = Alpha, alpha-disubstituted non-natural amino acid with an olefinic side chain
  VARIANT 15: note = Alpha, alpha-disubstituted non-natural amino acid with an olefinic side chain
  SITE 8..15: note = Residues are crosslinked
FDASISQXNEKINQXLAFIRKSDELLHNV

SEQ ID NO: 35 - protein, 29 aa, synthetic construct
  VARIANT 9: note = Alpha, alpha-disubstituted non-natural amino acid with an olefinic side chain
  VARIANT 16: note = Alpha, alpha-disubstituted non-natural amino acid with an olefinic side chain
  SITE 9..16: note = Residues are crosslinked
FDASISQVXEKINQSXAFIRKSDELLHNV

SEQ ID NO: 36 - protein, 29 aa, synthetic construct
  VARIANT 10: note = Alpha, alpha-disubstituted non-natural amino acid with an olefinic side chain
  VARIANT 17: note = Alpha, alpha-disubstituted non-natural amino acid with an olefinic side chain
  SITE 10..17: note = Residues are crosslinked
FDASISQVNXKINQSLXFIRKSDELLHNV

SEQ ID NO: 37 - protein, 29 aa, synthetic construct
  VARIANT 11: note = Alpha, alpha-disubstituted non-natural amino acid with an olefinic side chain
  VARIANT 18: note = Alpha, alpha-disubstituted non-natural amino acid with an olefinic side chain
  SITE 11..18: note = Residues are crosslinked
FDASISQVNEXINQSLAXIRKSDELLHNV

SEQ ID NO: 38 - protein, 29 aa, synthetic construct
  VARIANT 12: note = Alpha, alpha-disubstituted non-natural amino acid with an olefinic side chain
  VARIANT 19: note = Alpha, alpha-disubstituted non-natural amino acid with an olefinic side chain
  SITE 12..19: note = Residues are crosslinked
FDASISQVNEKXNQSLAFXRKSDELLHNV

SEQ ID NO: 39 - protein, 29 aa, synthetic construct
  VARIANT 13: note = Alpha, alpha-disubstituted non-natural amino acid with an olefinic side chain
  VARIANT 20: note = Alpha, alpha-disubstituted non-natural amino acid with an olefinic side chain
  SITE 13..20: note = Residues are crosslinked
FDASISQVNEKIXQSLAFIXKSDELLHNV

SEQ ID NO: 40 - protein, 29 aa, synthetic construct
  VARIANT 14: note = Alpha, alpha-disubstituted non-natural amino acid with an olefinic side chain
  VARIANT 21: note = Alpha, alpha-disubstituted non-natural amino acid with an olefinic side chain
  SITE 14..21: note = Residues are crosslinked
FDASISQVNEKINXSLAFIRXSDELLHNV

SEQ ID NO: 41 - protein, 29 aa, synthetic construct
  VARIANT 15: note = Alpha, alpha-disubstituted non-natural amino acid with an olefinic side chain
  VARIANT 22: note = Alpha, alpha-disubstituted non-natural amino acid with an olefinic side chain
  SITE 15..22: note = Residues are crosslinked
FDASISQVNEKINQXLAFIRKXDELLHNV

SEQ ID NO: 42 - protein, 29 aa, synthetic construct
  VARIANT 16: note = Alpha, alpha-disubstituted non-natural amino acid with an olefinic side chain
  VARIANT 23: note = Alpha, alpha-disubstituted non-natural amino acid with an olefinic side chain
  SITE 16..23: note = Residues are crosslinked
FDASISQVNEKINQSXAFIRKSXELLHNV

SEQ ID NO: 43 - protein, 29 aa, synthetic construct
  VARIANT 17: note = Alpha, alpha-disubstituted non-natural amino acid with an olefinic side chain
  VARIANT 24: note = Alpha, alpha-disubstituted non-natural amino acid with an olefinic side chain
  SITE 17..24: note = Residues are crosslinked
FDASISQVNEKINQSLXFIRKSDXLLHNV

SEQ ID NO: 44 - protein, 29 aa, synthetic construct
  VARIANT 18: note = Alpha, alpha-disubstituted non-natural amino acid with an olefinic side chain
  VARIANT 25: note = Alpha, alpha-disubstituted non-natural amino acid with an olefinic side chain
  SITE 18..25: note = Residues are crosslinked
FDASISQVNEKINQSLAXIRKSDEXLHNV

SEQ ID NO: 45 - protein, 29 aa, synthetic construct
  VARIANT 19: note = Alpha, alpha-disubstituted non-natural amino acid with an olefinic side chain
  VARIANT 26: note = Alpha, alpha-disubstituted non-natural amino acid with an olefinic side chain
  SITE 19..26: note = Residues are crosslinked
FDASISQVNEKINQSLAFXRKSDELXHNV

SEQ ID NO: 46 - protein, 29 aa, synthetic construct
  VARIANT 20: note = Alpha, alpha-disubstituted non-natural amino acid with an olefinic side chain
  VARIANT 27: note = Alpha, alpha-disubstituted non-natural amino acid with an olefinic side chain
  SITE 20..27: note = Residues are crosslinked
FDASISQVNEKINQSLAFIXKSDELLXNV

SEQ ID NO: 47 - protein, 29 aa, synthetic construct
  VARIANT 21: note = Alpha, alpha-disubstituted non-natural amino acid with an olefinic side chain
  VARIANT 28: note = Alpha, alpha-disubstituted non-natural amino acid with an olefinic side chain
  SITE 21..28: note = Residues are crosslinked
FDASISQVNEKINQSLAFIRXSDELLHXV

SEQ ID NO: 48 - protein, 29 aa, synthetic construct
  VARIANT 22: note = Alpha, alpha-disubstituted non-natural amino acid with an olefinic side chain
  VARIANT 29: note = Alpha, alpha-disubstituted non-natural amino acid with an olefinic side chain
  SITE 22..29: note = Residues are crosslinked
FDASISQVNEKINQSLAFIRKXDELLHNX

SEQ ID NO: 49 - protein, 29 aa, synthetic construct
  SITE 1: note = (R)-alpha-(7'-octenyl)alanine
  SITE 8: note = (S)-alpha-(4'-pentenyl)alanine
XDASISQXNEKINQSLAFIRKSDELLHNV

SEQ ID NO: 50 - protein, 29 aa, synthetic construct
  SITE 2: note = (R)-alpha-(7'-octenyl)alanine
  SITE 9: note = (S)-alpha-(4'-pentenyl)alanine
FXASISQVXEKINQSLAFIRKSDELLHNV

SEQ ID NO: 51 - protein, 29 aa, synthetic construct
  SITE 3: note = (R)-alpha-(7'-octenyl)alanine
  SITE 10: note = (S)-alpha-(4'-pentenyl)alanine
FDXSISQVNXKINQSLAFIRKSDELLHNV

SEQ ID NO: 52 - protein, 29 aa, synthetic construct
  SITE 4: note = (R)-alpha-(7'-octenyl)alanine
  SITE 11: note = (S)-alpha-(4'-pentenyl)alanine
FDAXISQVNEXINQSLAFIRKSDELLHNV

SEQ ID NO: 53 - protein, 29 aa, synthetic construct
  SITE 5: note = (R)-alpha-(7'-octenyl)alanine
  SITE 12: note = (S)-alpha-(4'-pentenyl)alanine
FDASXSQVNEKXNQSLAFIRKSDELLHNV

SEQ ID NO: 54 - protein, 29 aa, synthetic construct
  SITE 6: note = (R)-alpha-(7'-octenyl)alanine
  SITE 13: note = (S)-alpha-(4'-pentenyl)alanine
FDASIXQVNEKIXQSLAFIRKSDELLHNV

SEQ ID NO: 55 - protein, 29 aa, synthetic construct
  SITE 7: note = (R)-alpha-(7'-octenyl)alanine
  SITE 14: note = (S)-alpha-(4'-pentenyl)alanine
FDASISXVNEKINXSLAFIRKSDELLHNV

SEQ ID NO: 56 - protein, 29 aa, synthetic construct
  SITE 8: note = (R)-alpha-(7'-octenyl)alanine
  SITE 15: note = (S)-alpha-(4'-pentenyl)alanine
FDASISQXNEKINQXLAFIRKSDELLHNV

SEQ ID NO: 57 - protein, 29 aa, synthetic construct
  SITE 9: note = (R)-alpha-(7'-octenyl)alanine
  SITE 16: note = (S)-alpha-(4'-pentenyl)alanine
FDASISQVXEKINQSXAFIRKSDELLHNV

SEQ ID NO: 58 - protein, 29 aa, synthetic construct
  SITE 10: note = (R)-alpha-(7'-octenyl)alanine
  SITE 17: note = (S)-alpha-(4'-pentenyl)alanine
FDASISQVNXKINQSLXFIRKSDELLHNV

SEQ ID NO: 59 - protein, 29 aa, synthetic construct
  SITE 11: note = (R)-alpha-(7'-octenyl)alanine
  SITE 18: note = (S)-alpha-(4'-pentenyl)alanine
FDASISQVNEXINQSLAXIRKSDELLHNV

SEQ ID NO: 60 - protein, 29 aa, synthetic construct
  SITE 12: note = (R)-alpha-(7'-octenyl)alanine
  SITE 19: note = (S)-alpha-(4'-pentenyl)alanine
FDASISQVNEKXNQSLAFXRKSDELLHNV

SEQ ID NO: 61 - protein, 29 aa, synthetic construct
  SITE 13: note = (R)-alpha-(7'-octenyl)alanine
  SITE 20: note = (S)-alpha-(4'-pentenyl)alanine
FDASISQVNEKIXQSLAFIXKSDELLHNV

SEQ ID NO: 62 - protein, 29 aa, synthetic construct
  SITE 14: note = (R)-alpha-(7'-octenyl)alanine
  SITE 21: note = (S)-alpha-(4'-pentenyl)alanine
FDASISQVNEKINXSLAFIRXSDELLHNV

SEQ ID NO: 63 - protein, 29 aa, synthetic construct
  SITE 15: note = (R)-alpha-(7'-octenyl)alanine
  SITE 22: note = (S)-alpha-(4'-pentenyl)alanine
FDASISQVNEKINQXLAFIRKXDELLHNV

SEQ ID NO: 64 - protein, 29 aa, synthetic construct
  SITE 16: note = (R)-alpha-(7'-octenyl)alanine
  SITE 23: note = (S)-alpha-(4'-pentenyl)alanine
FDASISQVNEKINQSXAFIRKSXELLHNV

SEQ ID NO: 65 - protein, 29 aa, synthetic construct
  SITE 17: note = (R)-alpha-(7'-octenyl)alanine
  SITE 24: note = (S)-alpha-(4'-pentenyl)alanine
FDASISQVNEKINQSLXFIRKSDXLLHNV

SEQ ID NO: 66 - protein, 29 aa, synthetic construct
  SITE 18: note = (R)-alpha-(7'-octenyl)alanine
  SITE 25: note = (S)-alpha-(4'-pentenyl)alanine
FDASISQVNEKINQSLAXIRKSDEXLHNV

SEQ ID NO: 67 - protein, 29 aa, synthetic construct
  SITE 19: note = (R)-alpha-(7'-octenyl)alanine
  SITE 26: note = (S)-alpha-(4'-pentenyl)alanine
FDASISQVNEKINQSLAFXRKSDELXHNV

SEQ ID NO: 68 - protein, 29 aa, synthetic construct
  SITE 20: note = (R)-alpha-(7'-octenyl)alanine
  SITE 27: note = (S)-alpha-(4'-pentenyl)alanine
FDASISQVNEKINQSLAFIXKSDELLXNV

SEQ ID NO: 69 - protein, 29 aa, synthetic construct
  SITE 21: note = (R)-alpha-(7'-octenyl)alanine
  SITE 28: note = (S)-alpha-(4'-pentenyl)alanine
FDASISQVNEKINQSLAFIRXSDELLHXV

SEQ ID NO: 70 - protein, 29 aa, synthetic construct
  SITE 22: note = (R)-alpha-(7'-octenyl)alanine
  SITE 29: note = (S)-alpha-(4'-pentenyl)alanine
FDASISQVNEKINQSLAFIRKXDELLHNX

SEQ ID NO: 71 - protein, 30 aa, synthetic construct
  VARIANT 1: note = Alpha, alpha-disubstituted non-natural amino acid with an olefinic side chain
  VARIANT 8: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 1..8: note = Residues are crosslinked
XDASISQXNEKINQSLAFIRKSDELLHNVX

SEQ ID NO: 72 - protein, 30 aa, synthetic construct
  VARIANT 2: note = Alpha, alpha-disubstituted non-natural amino acid with an olefinic side chain
  VARIANT 9: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 2..9: note = Residues are crosslinked
FXASISQVXEKINQSLAFIRKSDELLHNVX

SEQ ID NO: 73 - protein, 30 aa, synthetic construct
  VARIANT 3: note = Alpha, alpha-disubstituted non-natural amino acid with an olefinic side chain
  VARIANT 10: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 3..10: note = Residues are crosslinked
FDXSISQVNXKINQSLAFIRKSDELLHNVX

SEQ ID NO: 74 - protein, 30 aa, synthetic construct
  VARIANT 4: note = Alpha, alpha-disubstituted non-natural amino acid with an olefinic side chain
  VARIANT 11: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 4..11: note = Residues are crosslinked
FDAXISQVNEXINQSLAFIRKSDELLHNVX

SEQ ID NO: 75 - protein, 30 aa, synthetic construct
  VARIANT 5: note = Alpha, alpha-disubstituted non-natural amino acid with an olefinic side chain
  VARIANT 12: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 5..12: note = Residues are crosslinked
FDASXSQVNEKXNQSLAFIRKSDELLHNVX

SEQ ID NO: 76 - protein, 30 aa, synthetic construct
  VARIANT 6: note = Alpha, alpha-disubstituted non-natural amino acid with an olefinic side chain
  VARIANT 13: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 6..13: note = Residues are crosslinked
FDASIXQVNEKIXQSLAFIRKSDELLHNVX

SEQ ID NO: 77 - protein, 30 aa, synthetic construct
  VARIANT 7: note = Alpha, alpha-disubstituted non-natural amino acid with an olefinic side chain
  VARIANT 14: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 7..14: note = Residues are crosslinked
FDASISXVNEKINXSLAFIRKSDELLHNVX

SEQ ID NO: 78 - protein, 30 aa, synthetic construct
  VARIANT 8: note = Alpha, alpha-disubstituted non-natural amino acid with an olefinic side chain
  VARIANT 15: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 8..15: note = Residues are crosslinked
FDASISQXNEKINQXLAFIRKSDELLHNVX

SEQ ID NO: 79 - protein, 30 aa, synthetic construct
  VARIANT 9: note = Alpha, alpha-disubstituted non-natural amino acid with an olefinic side chain
  VARIANT 16: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 9..16: note = Residues are crosslinked
FDASISQVXEKINQSXAFIRKSDELLHNVX

SEQ ID NO: 80 - protein, 30 aa, synthetic construct
  VARIANT 10: note = Alpha, alpha-disubstituted non-natural amino acid with an olefinic side chain
  VARIANT 17: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 10..17: note = Residues are crosslinked
FDASISQVNXKINQSLXFIRKSDELLHNVX

SEQ ID NO: 81 - protein, 30 aa, synthetic construct
  VARIANT 11: note = Alpha, alpha-disubstituted non-natural amino acid with an olefinic side chain
  VARIANT 18: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 11..18: note = Residues are crosslinked
FDASISQVNEXINQSLAXIRKSDELLHNVX

SEQ ID NO: 82 - protein, 30 aa, synthetic construct
  VARIANT 12: note = Alpha, alpha-disubstituted non-natural amino acid with an olefinic side chain
  VARIANT 19: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 12..19: note = Residues are crosslinked
FDASISQVNEKXNQSLAFXRKSDELLHNVX

SEQ ID NO: 83 - protein, 30 aa, synthetic construct
  VARIANT 13: note = Alpha, alpha-disubstituted non-natural amino acid with an olefinic side chain
  VARIANT 20: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 13..20: note = Residues are crosslinked
FDASISQVNEKIXQSLAFIXKSDELLHNVX

SEQ ID NO: 84 - protein, 30 aa, synthetic construct
  VARIANT 14: note = Alpha, alpha-disubstituted non-natural amino acid with an olefinic side chain
  VARIANT 21: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 14..21: note = Residues are crosslinked
FDASISQVNEKINXSLAFIRXSDELLHNVX

SEQ ID NO: 85 - protein, 30 aa, synthetic construct
  VARIANT 15: note = Alpha, alpha-disubstituted non-natural amino acid with an olefinic side chain
  VARIANT 22: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 15..22: note = Residues are crosslinked
FDASISQVNEKINQXLAFIRKXDELLHNVX

SEQ ID NO: 86 - protein, 30 aa, synthetic construct
  VARIANT 16: note = Alpha, alpha-disubstituted non-natural amino acid with an olefinic side chain
  VARIANT 23: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 16..23: note = Residues are crosslinked
FDASISQVNEKINQSXAFIRKSXELLHNVX

SEQ ID NO: 87 - protein, 30 aa, synthetic construct
  VARIANT 17: note = Alpha, alpha-disubstituted non-natural amino acid with an olefinic side chain
  VARIANT 24: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 17..24: note = Residues are crosslinked
FDASISQVNEKINQSLXFIRKSDXLLHNVX

SEQ ID NO: 88 - protein, 30 aa, synthetic construct
  VARIANT 18: note = Alpha, alpha-disubstituted non-natural amino acid with an olefinic side chain
  VARIANT 25: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 18..25: note = Residues are crosslinked
FDASISQVNEKINQSLAXIRKSDEXLHNVX

SEQ ID NO: 89 - protein, 30 aa, synthetic construct
  VARIANT 19: note = Alpha, alpha-disubstituted non-natural amino acid with an olefinic side chain
  VARIANT 26: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 19..26: note = Residues are crosslinked
FDASISQVNEKINQSLAFXRKSDELXHNVX

SEQ ID NO: 90 - protein, 30 aa, synthetic construct
  VARIANT 20: note = Alpha, alpha-disubstituted non-natural amino acid with an olefinic side chain
  VARIANT 27: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 20..27: note = Residues are crosslinked
FDASISQVNEKINQSLAFIXKSDELLXNVX

SEQ ID NO: 91 - protein, 30 aa, synthetic construct
  VARIANT 21: note = Alpha, alpha-disubstituted non-natural amino acid with an olefinic side chain
  VARIANT 28: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 21..28: note = Residues are crosslinked
FDASISQVNEKINQSLAFIRXSDELLHXVX

SEQ ID NO: 92 - protein, 30 aa, synthetic construct
  VARIANT 22: note = Alpha, alpha-disubstituted non-natural amino acid with an olefinic side chain
  VARIANT 29: note = Alpha, alpha-disubstituted non-natural amino acid with an olefinic side chain
  VARIANT 30: note = Any diamino acid modified with 1-36x polyethylene glycol units attached to cholesterol or thiocholesterol
  SITE 22..29: note = Residues are crosslinked
FDASISQVNEKINQSLAFIRKXDELLHNXX

SEQ ID NO: 93 - protein, 60 aa, unidentified
SKVLHLEGEVNKIKNALLSTNKAVVSLSNGVSVLTSKVLDLKNYINNRLLPIVNQQSCRI

SEQ ID NO: 94 - protein, 60 aa, unidentified
SKVLHLEGEVNKIKNALLSTNKAVVSLSNGVSVLTSKVLDLKNYINNQLLPIVNQQSCRI

SEQ ID NO: 95 - protein, 60 aa, unidentified
SKVLHLEGEVNKIKSALLSTNKAVVSLSNGVSVLTSKVLDLKNYIDKQLLPIVNKQSCRI

SEQ ID NO: 96 - protein, 49 aa, unidentified
NFYDPLVFPSDEFDASISQVNEKINQSLAFIRKSDELLHHVNAGKSTTN

SEQ ID NO: 97 - protein, 49 aa, unidentified
NYYDPLVFPSDEFDASISQVNEKINQSLAFIRRSDELLHNVNTGKSTTN

SEQ ID NO: 98 - protein, 60 aa, synthetic construct
  VARIANT 15: note = S or N
  VARIANT 46: note = D or N
  VARIANT 47: note = K or N
  VARIANT 48: note = Q or R
  VARIANT 55: note = K or Q
  VARIANT 59: note = S or R
SKVLHLEGEVNKIKXALLSTNKAVVSLSNGVSVLTSKVLDLKNYIXXXLLPIVNXQSCXI

SEQ ID NO: 99 - protein, 49 aa, synthetic construct
  VARIANT 2: note = F or Y
  VARIANT 33: note = K or R
  VARIANT 40: note = N or H
  VARIANT 43: note = A or T
NXYDPLVFPSDEFDASISQVNEKINQSLAFIRXSDELLHXVNXGKSTTN

SEQ ID NO: 100 - protein, 29 aa, synthetic construct
FDASISQVNEKINQSLAFIRKSDELLHNV

SEQ ID NO: 101-199 - skipped (99 entries)

SEQ ID NO: 200 - protein, 4 aa, synthetic construct
FDAS

SEQ ID NO: 201 - protein, 5 aa, synthetic construct
FDASI

SEQ ID NO: 202 - protein, 6 aa, synthetic construct
FDASIS

SEQ ID NO: 203 - protein, 7 aa, synthetic construct
FDASISQ

SEQ ID NO: 204 - protein, 8 aa, synthetic construct
FDASISQV

SEQ ID NO: 205 - protein, 9 aa, synthetic construct
FDASISQVN

SEQ ID NO: 206 - protein, 10 aa, synthetic construct
FDASISQVNE

SEQ ID NO: 207 - protein, 11 aa, synthetic construct
FDASISQVNEK

SEQ ID NO: 208 - protein, 12 aa, synthetic construct
FDASISQVNEKI

SEQ ID NO: 209 - protein, 13 aa, synthetic construct
FDASISQVNEKIN

SEQ ID NO: 210 - protein, 14 aa, synthetic construct
FDASISQVNEKINQ

SEQ ID NO: 211 - protein, 15 aa, synthetic construct
FDASISQVNEKINQS

SEQ ID NO: 212 - protein, 16 aa, synthetic construct
FDASISQVNEKINQSL

SEQ ID NO: 213 - protein, 17 aa, synthetic construct
FDASISQVNEKINQSLA

SEQ ID NO: 214 - protein, 18 aa, synthetic construct
FDASISQVNEKINQSLAF

SEQ ID NO: 215 - protein, 19 aa, synthetic construct
FDASISQVNEKINQSLAFI

SEQ ID NO: 216 - protein, 20 aa, synthetic construct
FDASISQVNEKINQSLAFIR

SEQ ID NO: 217 - protein, 21 aa, synthetic construct
FDASISQVNEKINQSLAFIRK

SEQ ID NO: 218 - protein, 6 aa, synthetic construct
DASISQ

SEQ ID NO: 219 - protein, 6 aa, synthetic construct
ASISQV

SEQ ID NO: 220 - protein, 6 aa, synthetic construct
SISQVN

SEQ ID NO: 221 - protein, 6 aa, synthetic construct
ISQVNE

SEQ ID NO: 222 - protein, 6 aa, synthetic construct
SQVNEK

SEQ ID NO: 223 - protein, 6 aa, synthetic construct
QVNEKI

SEQ ID NO: 224 - protein, 6 aa, synthetic construct
VNEKIN

SEQ ID NO: 225 - protein, 6 aa, synthetic construct
NEKINQ

SEQ ID NO: 226 - protein, 6 aa, synthetic construct
EKINQS

SEQ ID NO: 227 - protein, 6 aa, synthetic construct
KINQSL

SEQ ID NO: 228 - protein, 6 aa, synthetic construct
INQSLA

SEQ ID NO: 229 - protein, 6 aa, synthetic construct
NQSLAF

SEQ ID NO: 230 - protein, 6 aa, synthetic construct
QSLAFI

SEQ ID NO: 231 - protein, 6 aa, synthetic construct
SLAFIR

SEQ ID NO: 232 - protein, 6 aa, synthetic construct
LAFIRK

SEQ ID NO: 233 - protein, 6 aa, synthetic construct
AFIRKS

SEQ ID NO: 234 - protein, 6 aa, synthetic construct
FIRKSD

SEQ ID NO: 235 - protein, 6 aa, synthetic construct
IRKSDE

SEQ ID NO: 236 - protein, 6 aa, synthetic construct
RKSDEL

SEQ ID NO: 237 - protein, 6 aa, synthetic construct
KSDELL

SEQ ID NO: 238 - protein, 6 aa, synthetic construct
SDELLH

SEQ ID NO: 239 - protein, 6 aa, synthetic construct
DELLHN

SEQ ID NO: 240 - protein, 21 aa, synthetic construct
NEKINQSLAFIRKSDELLHNV

SEQ ID NO: 241 - protein, 20 aa, synthetic construct
EKINQSLAFIRKSDELLHNV

SEQ ID NO: 242 - protein, 19 aa, synthetic construct
KINQSLAFIRKSDELLHNV

SEQ ID NO: 243 - protein, 18 aa, synthetic construct
INQSLAFIRKSDELLHNV

SEQ ID NO: 244 - protein, 17 aa, synthetic construct
NQSLAFIRKSDELLHNV

SEQ ID NO: 245 - protein, 16 aa, synthetic construct
QSLAFIRKSDELLHNV

SEQ ID NO: 246 - protein, 15 aa, synthetic construct
SLAFIRKSDELLHNV

SEQ ID NO: 247 - protein, 14 aa, synthetic construct
LAFIRKSDELLHNV

SEQ ID NO: 248 - protein, 13 aa, synthetic construct
AFIRKSDELLHNV

SEQ ID NO: 249 - protein, 12 aa, synthetic construct
FIRKSDELLHNV

SEQ ID NO: 250 - protein, 11 aa, synthetic construct
IRKSDELLHNV

SEQ ID NO: 251 - protein, 10 aa, synthetic construct
RKSDELLHNV

SEQ ID NO: 252 - protein, 9 aa, synthetic construct
KSDELLHNV

SEQ ID NO: 253 - protein, 8 aa, synthetic construct
SDELLHNV

SEQ ID NO: 254 - protein, 7 aa, synthetic construct
DELLHNV

SEQ ID NO: 255 - protein, 6 aa, synthetic construct
ELLHNV

SEQ ID NO: 256 - protein, 5 aa, synthetic construct
LLHNV

SEQ ID NO: 257 - protein, 4 aa, synthetic construct
LHNV

SEQ ID NO: 258 - protein, 6 aa, synthetic construct
NAGKST

SEQ ID NO: 259 - protein, 5 aa, synthetic construct
NAGKS

SEQ ID NO: 260 - protein, 4 aa, synthetic construct
NAGK

SEQ ID NO: 261 - protein, 30 aa, synthetic construct; identical to SEQ ID NO: 5
  SITE 3: note = (R)-alpha-(7'-octenyl)alanine
  SITE 10: note = (S)-alpha-(4'-pentenyl)alanine
  VARIANT 30: note = Lysine modified with 1-36x polyethylene glycol units attached to thiocholesterol or cholesterol
  SITE 3..10: note = Residues are crosslinked

SEQ ID NO: 262 - protein, 30 aa, synthetic construct; identical to SEQ ID NO: 5
  SITE 1: note = Fluorenylmethoxycarbonyl protecting group modified residue
  SITE 3: note = (R)-alpha-(7'-octenyl)alanine
  SITE 10: note = (S)-alpha-(4'-pentenyl)alanine
  SITE 30: note = Lys-N-epsilon-1-(4,4-dimethyl-2,6-dioxocyclohex-1-y lidene)ethyl

SEQ ID NO: 263 - protein, 30 aa, synthetic construct; identical to SEQ ID NO: 5
  SITE 1: note = Acetylated residue
  SITE 3: note = (R)-alpha-(7'-octenyl)alanine
  SITE 10: note = (S)-alpha-(4'-pentenyl)alanine
  SITE 30: note = Residue attached to 4x polyethylene glycol with fluorenylmethoxycarbonyl protecting group

SEQ ID NO: 264 - protein, 30 aa, synthetic construct; identical to SEQ ID NO: 5
  SITE 1: note = Acetylated residue
  SITE 3: note = (R)-alpha-(7'-octenyl)alanine
  SITE 10: note = (S)-alpha-(4'-pentenyl)alanine
  SITE 30: note = Residue attached to 4x polyethylene glycol thiocholesterol

SEQ ID NO: 265 - protein, 30 aa, synthetic construct; identical to SEQ ID NO: 6
  SITE 1: note = (R)-alpha-(7'-octenyl)alanine
  SITE 8: note = (S)-alpha-(4'-pentenyl)alanine
  VARIANT 30: note = Lysine modified with 1-36x polyethylene glycol units attached to thiocholesterol or cholesterol
  SITE 1..8: note = Residues are crosslinked

SEQ ID NO: 266 - protein, 30 aa, synthetic construct; identical to SEQ ID NO: 21
  SITE 17: note = (R)-alpha-(7'-octenyl)alanine
  SITE 24: note = (S)-alpha-(4'-pentenyl)alanine
  VARIANT 30: note = Lysine modified with 1-36x polyethylene glycol units attached to thiocholesterol or cholesterol
  SITE 17..24: note = Residues are crosslinked

SEQ ID NO: 267 - protein, 30 aa, synthetic construct; identical to SEQ ID NO: 5
  SITE 3: note = (R)-alpha-(7'-octenyl)alanine
  SITE 10: note = (S)-alpha-(4'-pentenyl)alanine
  SITE 30: note = Residue attached to thiocholesterol

SEQ ID NO: 268 - protein, 30 aa, synthetic construct; identical to SEQ ID NO: 5
  SITE 3: note = (R)-alpha-(7'-octenyl)alanine
  SITE 10: note = (S)-alpha-(4'-pentenyl)alanine
  SITE 30: note = Residue attached to 4x polyethylene glycol thiocholesterol

SEQ ID NO: 269 - protein, 30 aa, synthetic construct; identical to SEQ ID NO: 5
  SITE 3: note = (R)-alpha-(7'-octenyl)alanine
  SITE 10: note = (S)-alpha-(4'-pentenyl)alanine
  SITE 30: note = Residue attached to 8x polyethylene glycol thiocholesterol

SEQ ID NO: 270 - protein, 30 aa, synthetic construct; identical to SEQ ID NO: 5
  SITE 3: note = (R)-alpha-(7'-octenyl)alanine
  SITE 10: note = (S)-alpha-(4'-pentenyl)alanine
  SITE 30: note = Residue attached to 12x polyethylene glycol thiocholesterol

SEQ ID NO: 271 - protein, 30 aa, synthetic construct; identical to SEQ ID NO: 5
  SITE 3: note = (R)-alpha-(7'-octenyl)alanine
  SITE 10: note = (S)-alpha-(4'-pentenyl)alanine
  SITE 30: note = Residue attached to 16x polyethylene glycol thiocholesterol

SEQ ID NO: 272 - protein, 30 aa, synthetic construct; identical to SEQ ID NO: 5
  SITE 3: note = (R)-alpha-(7'-octenyl)alanine
  SITE 10: note = (S)-alpha-(4'-pentenyl)alanine
  SITE 30: note = Residue attached to 20x polyethylene glycol thiocholesterol

SEQ ID NO: 273 - protein, 30 aa, synthetic construct; identical to SEQ ID NO: 21
  SITE 17: note = (R)-alpha-(7'-octenyl)alanine
  SITE 24: note = (S)-alpha-(4'-pentenyl)alanine
  SITE 30: note = Residue attached to thiocholesterol

SEQ ID NO: 274 - protein, 30 aa, synthetic construct; identical to SEQ ID NO: 21
  SITE 17: note = (R)-alpha-(7'-octenyl)alanine
  SITE 24: note = (S)-alpha-(4'-pentenyl)alanine
  SITE 30: note = Residue attached to 4x polyethylene glycol thiocholesterol

SEQ ID NO: 275 - protein, 30 aa, synthetic construct; identical to SEQ ID NO: 21
  SITE 17: note = (R)-alpha-(7'-octenyl)alanine
  SITE 24: note = (S)-alpha-(4'-pentenyl)alanine
  SITE 30: note = Residue attached to 8x polyethylene glycol thiocholesterol

SEQ ID NO: 276 - protein, 30 aa, synthetic construct; identical to SEQ ID NO: 21
  SITE 17: note = (R)-alpha-(7'-octenyl)alanine
  SITE 24: note = (S)-alpha-(4'-pentenyl)alanine
  SITE 30: note = Residue attached to 12x polyethylene glycol thiocholesterol

SEQ ID NO: 277 - protein, 30 aa, synthetic construct; identical to SEQ ID NO: 21
  SITE 17: note = (R)-alpha-(7'-octenyl)alanine
  SITE 24: note = (S)-alpha-(4'-pentenyl)alanine
  SITE 30: note = Residue attached to 16x polyethylene glycol thiocholesterol

SEQ ID NO: 278 - protein, 30 aa, synthetic construct; identical to SEQ ID NO: 21
  SITE 17: note = (R)-alpha-(7'-octenyl)alanine
  SITE 24: note = (S)-alpha-(4'-pentenyl)alanine
  SITE 30: note = Residue attached to 20x polyethylene glycol thiocholesterol

SEQ ID NO: 279 - protein, 6 aa, synthetic construct
HHHHHH

SEQ ID NO: 280 - protein, 32 aa, synthetic construct
SDEFDASISQVNEKINQSLAFIRKSDELLHNV

SEQ ID NO: 281 - protein, 32 aa, synthetic construct
  SITE 20: note = (R)-alpha-(7'-octenyl)alanine
  SITE 27: note = (S)-alpha-(4'-pentenyl)alanine
  SITE 32: note = Residue attached to a lipid via a linker
SDEFDASISQVNEKINQSLXFIRKSDXLLHNV

SEQ ID NO: 282 - protein, 32 aa, synthetic construct
  VARIANT 1: note = Any amino acid
  VARIANT 2: note = Any negatively charged amino acid
  VARIANT 3: note = Any negatively charged amino acid
XXXFDASISQVNEKINQSLAFIRKSDELLHNV

SEQ ID NO: 283 - protein, 29 aa, synthetic construct
  SITE 17: note = (R)-alpha-(7'-octenyl)alanine
  SITE 24: note = (S)-alpha-(4'-pentenyl)alanine
  SITE 29: note = Residue attached to a lipid via a linker
FDASISQVNEKINQSLXFIRKSDXLLHNV

SEQ ID NO: 284 - protein, 32 aa, synthetic construct
  VARIANT 1: note = Any amino acid
  VARIANT 2: note = Any negatively charged amino acid
  VARIANT 3: note = Any negatively charged amino acid
  SITE 20: note = (R)-alpha-(7'-octenyl)alanine
  SITE 27: note = (S)-alpha-(4'-pentenyl)alanine
XXXFDASISQVNEKINQSLXFIRKSDXLLHNV

SEQ ID NO: 285 - protein, 32 aa, synthetic construct; identical to SEQ ID NO: 281
  SITE 20: note = (R)-alpha-(7'-octenyl)alanine
  SITE 27: note = (S)-alpha-(4'-pentenyl)alanine

SEQ ID NO: 286 - protein, 32 aa, synthetic construct; identical to SEQ ID NO: 284
  VARIANT 1: note = Any amino acid
  VARIANT 2: note = Any negatively charged amino acid
  VARIANT 3: note = Any negatively charged amino acid
  SITE 20: note = (R)-alpha-(7'-octenyl)alanine
  SITE 27: note = (S)-alpha-(4'-pentenyl)alanine
  SITE 32: note = Residue attached to a lipid via a linker

(19) United States
(12) Patent Application Publication
(10) Pub. No.: US 2026/0041753 A1
(43) Pub. Date: Feb. 12, 2026

(54) RESPIRATORY SYNCYTIAL VIRUS SURFACE GLYCOPROTEIN PEPTIDES, CONJUGATES, AND USES THEREOF

(71) Applicant: Dana-Farber Cancer Institute, Inc., Boston, MA (US)

(72) Inventors: Gregory H. Bird, Pelham, NH (US); Loren D. Walensky, Newton, MA (US)

(21) Appl. No.: 19/162,474

(22) PCT Filed: Mar. 7, 2024
(86) PCT No.: PCT/US2024/018803

§ 371 Date: Sep. 5, 2025

Related U.S. Application Data
(60) Provisional application No. 63/489,096, filed on Mar. 8, 2023.

(51) Int. Cl. A61K 39/12; A61K 39/00; C07K 14/005

(52) U.S. Cl. CPC A61K 39/12; C07K 14/005; A61K 2039/60; A61K 2039/627; C12N 2760/18522; C12N 2760/18534

(57) ABSTRACT

This disclosure relates to structurally-stabilized (e.g., stapled, e.g., hydrocarbon stapled) respiratory syncytial virus (RSV) peptides and variants thereof, and structurally-stabilized (e.g., stapled, e.g., hydrocarbon stapled) RSV peptides and variants thereof, conjugated with polyethylene glycol (PEG) and/or cholesterol (or a variant thereof, e.g., thiocholesterol), e.g., a PEG(n)-cholesterol or PEG(n)-thiocholesterol derivatization, and methods for using such structurally-stabilized peptides and conjugates in the prevention and treatment of an RSV infection in a subject (e.g., human).

### CROSS-REFERENCE TO RELATED APPLICATION

This application claims the benefit of priority of U.S. Provisional Appl. No. 63/489,096, filed Mar. 8, 2023, the contents of which are incorporated by reference herein in their entirety.

### SEQUENCE LISTING

The instant application contains a Sequence Listing which has been submitted electronically in XML file format and is hereby incorporated by reference in its entirety. Said XML copy, created on Mar. 6, 2024, is named 00530-0418W01_SL.xml and is 332,258 bytes in size.

### TECHNICAL FIELD

This disclosure relates to structurally-stabilized (e.g., stapled, e.g., hydrocarbon stapled) respiratory syncytial virus (RSV) peptides and structurally-stabilized (e.g., stapled, e.g., hydrocarbon stapled) RSV peptides conjugated with polyethylene glycol (PEG) and/or cholesterol (or a variant thereof, e.g., thiocholesterol), e.g., a generated PEG(n)-cholesterol or PEG(n)-thiocholesterol derivatization and methods for using such structurally-stabilized peptide conjugates in the prevention and treatment of an RSV infection in a subject (e.g., human).

### BACKGROUND

Respiratory Syncytial Virus (RSV) infection causes 64 million cases of respiratory disease and 166,000 deaths annually worldwide. Drug resistant RSV strains have been reported (Adams et al., Clin. Infect. Disease, 51:185-188, 2010; Douglas et al., J. Virol., 49:2560-2466, 2005).

New and improved strategies for the prophylaxis and/or treatment of RSV infection are required.

### SUMMARY

This application relates to compositions and methods disclosing peptide stabilizing technology (e.g., stapling, e.g., hydrocarbon stapling) that recapitulates and fortifies the structure of bioactive helices. In some instances, the peptide stapling is combined with a method for cholesterol or a cholesterol variant (e.g., thiocholesterol) (e.g., PEG(n)-cholesterol or PEG(n)thiocholesterol) derivatization to generate an optimized and targeted prophylactic and therapeutic agent for prevention and/or treatment of RSV infection. By inserting “staples” (e.g., all-hydrocarbon staples) into RSV peptides, bioactive-helical structure can be restored and remarkable protease resistance can be conferred by burying the otherwise labile amide bonds at the core of the helical structure and/or restraining amide bonds in a manner that precludes their recognition and proteolysis by the body's proteases. Here, hydrocarbon-stapled and PEG(n)-cholesterol or PEG(n)thiocholesterol derivatized (hydrocarbon-stapled conjugates) peptide inhibitors of RSV are disclosed. These structurally-stabilized peptides and conjugates are used to prevent and/or treat RSV infection.

Provided herein is a conjugate comprising (i) a structurally-stabilized peptide and (ii) cholesterol or thiocholesterol; wherein the cholesterol or thiocholesterol are linked, directly or via a linker, to the C-terminal amino acid of the structurally-stabilized peptide; wherein the structurally-stabilized peptide comprises an internally cross-linked amino acid sequence comprising 25 to 29 contiguous amino acids of the sequence FDASISQVNEKINQSLAFIRKSDELLHNV (SEQ ID NO:100), except for two to six amino acid substitutions relative to the sequence of SEQ ID NO:100 and wherein the internally cross-linked amino acid sequence does not include the sequence NAGKST (SEQ ID NO:258);wherein two of the two to six amino acid substitutions are with α, α-disubstituted non-natural amino acids with olefinic side chains cross-linked to each other, wherein the α, α-disubstituted non-natural amino acids with olefinic side chains cross-linked to each other are separated by three or six amino acids; wherein the conjugate binds to an RSV 5-helix bundle protein and/or wherein the conjugate inhibits infection of a cell by RSV and/or prevents infection of a cell by RSV; and wherein the conjugate is 25 to 45 amino acids in length, optionally wherein the conjugate is 30 amino acids in length. In some instances, the two substitutions with α, α-disubstituted non-natural amino acids with olefinic side chains cross-linked to each other are at the amino acids corresponding to positions 1 and 8 of the sequence set forth in SEQ ID NO: 100, at the amino acids corresponding to 3 and 10 of the sequence set forth in SEQ ID NO:100, or at the amino acids corresponding to positions 17 and 24 of the sequence set forth in SEQ ID NO:100.

Also provided herein is a conjugate comprising (i) a structurally-stabilized peptide and (ii) cholesterol or thiocholesterol; wherein the cholesterol or thiocholesterol are linked, directly or via a linker, to the C-terminal amino acid of the structurally-stabilized peptide; wherein the structurally-stabilized peptide comprises an internally cross-linked amino acid sequence comprising the sequence X1X2X3FDASISQVNEKINQSLAFIRKSDELLHNV (SEQ ID NO: 282) [wherein X1 is any amino acid, optionally S; X2 and X3 are any negatively charged amino acid], except for two to six amino acid substitutions relative to the sequence of SEQ ID NO: 282 and wherein the internally cross-linked amino acid sequence does not include the sequence NAGKST (SEQ ID NO:258);wherein two of the two to six amino acid substitutions are with α, α-disubstituted non-natural amino acids with olefinic side chains cross-linked to each other, wherein the α, α-disubstituted non-natural amino acids with olefinic side chains cross-linked to each other are separated by three or six amino acids; wherein the conjugate binds to an RSV 5-helix bundle protein and/or wherein the conjugate inhibits infection of a cell by RSV and/or prevents infection of a cell by RSV; and wherein the conjugate is 32 to 45 amino acids in length, optionally wherein the conjugate is 32 amino acids in length. In some instances, the two substitutions with α, α-disubstituted non-natural amino acids with olefinic side chains cross-linked to each other are at the amino acids corresponding to positions 4 and 11 of the sequence set forth in SEQ ID NO: 282, at the amino acids corresponding to 6 and 13 of the sequence set forth in SEQ ID NO: 282, or at the amino acids corresponding to positions 20 and 27 of the sequence set forth in SEQ ID NO: 282. In one instance, the structurally-stabilized peptide comprises or consists of SDEFDASISQVNEKINQSLAFIRKSDELLHNV (SEQ ID NO:280) except for two to six amino acid substitutions relative to the sequence of SEQ ID NO:280 (e.g., amino acids corresponding to positions 4 and 11 of the sequence set forth in SEQ ID NO: 282; amino acids corresponding to 6 and 13 of the sequence set forth in SEQ ID NO: 282; or at the amino acids corresponding to positions 20 and 27 of the sequence set forth in SEQ ID NO: 282) and wherein the internally cross-linked amino acid sequence does not include the sequence NAGKST (SEQ ID NO:258); wherein two of the two to six amino acid substitutions are with α, α-disubstituted non-natural amino acids with olefinic side chains cross-linked to each other, wherein the α, α-disubstituted non-natural amino acids with olefinic side chains cross-linked to each other are separated by three or six amino acids; wherein the conjugate binds to an RSV 5-helix bundle protein and/or wherein the conjugate inhibits infection of a cell by RSV and/or prevents infection of a cell by RSV; and wherein the conjugate is 32 to 45 amino acids in length, optionally wherein the conjugate is 30 amino acids in length.

Also provided herein is a conjugate comprising (i) a structurally-stabilized peptide and (ii) cholesterol or thiocholesterol; wherein the cholesterol or thiocholesterol are linked, directly or via a linker, to the C-terminal amino acid of the structurally-stabilized peptide; wherein the structurally-stabilized peptide comprises an internally cross-linked amino acid sequence having the formula:

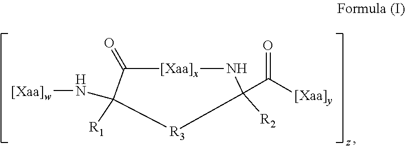

or a pharmaceutically acceptable salt thereof; wherein each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted; wherein x is 3 or 6; wherein each R₃ is independently alkylene, alkenylene, or alkynylene, any of which is substituted or unsubstituted; wherein z is 1, 2, 3, 4, 5, 6, 7, 8, 9, or 10; wherein the internally cross-linked amino sequence comprises 25 to 29 contiguous amino acids of the sequence of SEQ ID NO:100, except for two to six amino acid substitutions relative to the sequence of SEQ ID NO:100 and wherein the internally cross-linked amino acid sequence does not include the sequence NAGKST (SEQ ID NO:258); wherein the conjugate binds to an RSV 5-helix bundle protein and/or wherein the conjugate inhibits infection of a cell by RSV and/or prevents infection of a cell by RSV; and wherein the conjugate is 25 to 45 amino acids in length, optionally wherein the conjugate is 30 amino acids in length. In some instances, R₃ is an internal cross-link between the amino acids corresponding to positions 1 and 8 of the sequence set forth in SEQ ID NO:100, between the amino acids corresponding to positions 3 and 10 of the sequence set forth in SEQ ID NO:100, or between the positions 17 and 24 of the sequence set forth in SEQ ID NO:100 In some instances, the internally cross-linked amino acid sequence further comprises or consists of the sequence X1X2X3 wherein X1 is any amino acid, optionally S; X2 and X3 are any negatively charged amino acid immediately upstream of the first amino acid of SEQ ID NO:100 (i.e., immediately upstream of the first F of SEQ ID NO:100). In some cases X1X2X3=SDE. In one instance, the amino acid sequence comprises or consists of SDEFDASISQVNEKINQSLAFIRKSDELLHNV (SEQ ID NO:280) except for two to six amino acid substitutions relative to the sequence of SEQ ID NO:280 and wherein the internally cross-linked amino acid sequence does not include the sequence NAGKST (SEQ ID NO:258); wherein two of the two to six amino acid substitutions are with α, α-disubstituted non-natural amino acids with olefinic side chains cross-linked to each other, wherein the α, α-disubstituted non-natural amino acids with olefinic side chains cross-linked to each other are separated by three or six amino acids; wherein the conjugate binds to an RSV 5-helix bundle protein and/or wherein the conjugate inhibits infection of a cell by RSV and/or prevents infection of a cell by RSV; and wherein the conjugate is 32 to 45 amino acids in length, optionally wherein the conjugate is 30 amino acids in length.

In some instances of the foregoing conjugates, the conjugate comprises the cholesterol. In some instances, the conjugate comprises the cholesterol and the linker comprises PEG. In some instances, the conjugate comprises PEG(n)-cholesterol directly linked to the C-terminal amino acid of the structurally-stabilized peptide, wherein n is 1-36, optionally wherein n is 4, 5, 6, 7, 8, 12, 16, or 20. In some instances, the conjugate comprises Formula II directly linked to the C-terminal amino acid of the structurally-stabilized peptide:

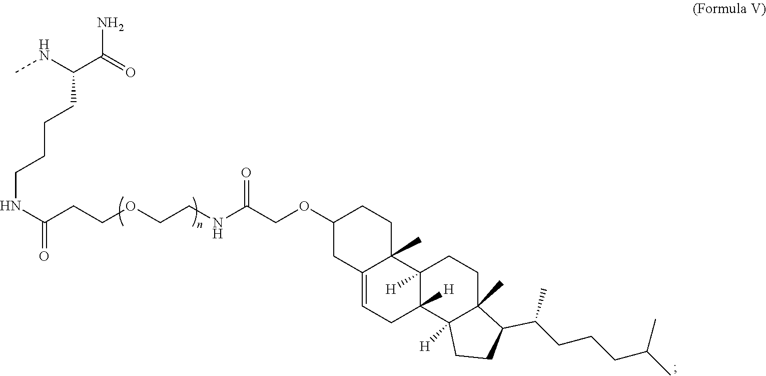

wherein n is 1-36, optionally wherein n is 4, 5, 6, 7, 8, 12, 16, or 20.

In some instances of the foregoing conjugates, the conjugate comprises the thiocholesterol. In some instances, the conjugate comprises the thiocholesterol and the linker comprises PEG. In some instances, the conjugate comprises PEG(n)-thiocholesterol directly linked to the C-terminal amino acid of the structurally-stabilized peptide, wherein n is 1-36, optionally wherein n is 4, 5, 6, 7, 8, 12, 16, or 20. In some instances, the conjugate comprises Formula III directly linked to the C-terminal amino acid of the structurally-stabilized peptide:

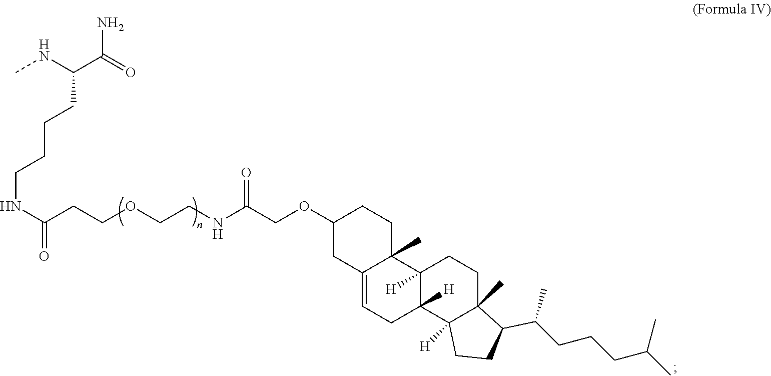

wherein n is 1-36, optionally wherein n is 4, 5, 6, 7, 8, 12, 16, or 20.

In some instances of the foregoing conjugates, the α, α-disubstituted non-natural amino acids with olefinic side chains cross-linked to each other are separated by three amino acids, optionally wherein each of the α, α-disubstituted non-natural amino acids with olefinic side chains cross-linked to each other is (S)-α-(4′-pentenyl)alanine.

In some instances of the foregoing conjugates, the α, α-disubstituted non-natural amino acids with olefinic side chains cross-linked to each other are separated by six amino acids, optionally wherein the α, α-disubstituted non-natural amino acids with olefinic side chains cross-linked to each other are (R)-α-(7′-octenyl)alanine and (S)-α-(4′-pentenyl)alanine.

In some instances of the foregoing conjugates, the structurally-stabilized peptide comprises the sequence set forth in any one of SEQ ID NOs:27, 29, 36, 43, and 46. In some instances of the foregoing conjugates, the structurally-stabilized peptide comprises the sequence set forth in any one of SEQ ID NOs:49, 51, 58, 65, and 68.

In some instances of the foregoing conjugates, the conjugate comprises the sequence set forth in any one of SEQ ID NOs: 5-26 and 71-92. In some instances, the conjugate comprises the sequence set forth in any one of SEQ ID NOs:71, 73, 80, 87, and 90. In some instances, the conjugate comprises the sequence set forth in any one of SEQ ID NOs:5, 6, 14, 21, and 24. In some instances, the conjugate comprises the sequence set forth in SEQ ID NO:21 or 281.

In some instances of the foregoing conjugates, the conjugate comprises an internally cross-linked peptide that is 25 to 34, 26 to 33, 27 to 32, 28 to 31, 29, 30, 31, or 32 amino acids in length.

Also provided herein is a conjugate comprising or consisting of 8DASISQXNEKINQSLAFIRKSDELLHNV* (SEQ ID NO:265), wherein 8 is internally cross-linked to X, wherein 8 is (R)-α-(7′-octenyl)alanine, wherein X is (S)-α-(4′-pentenyl)alanine, wherein * is the formula

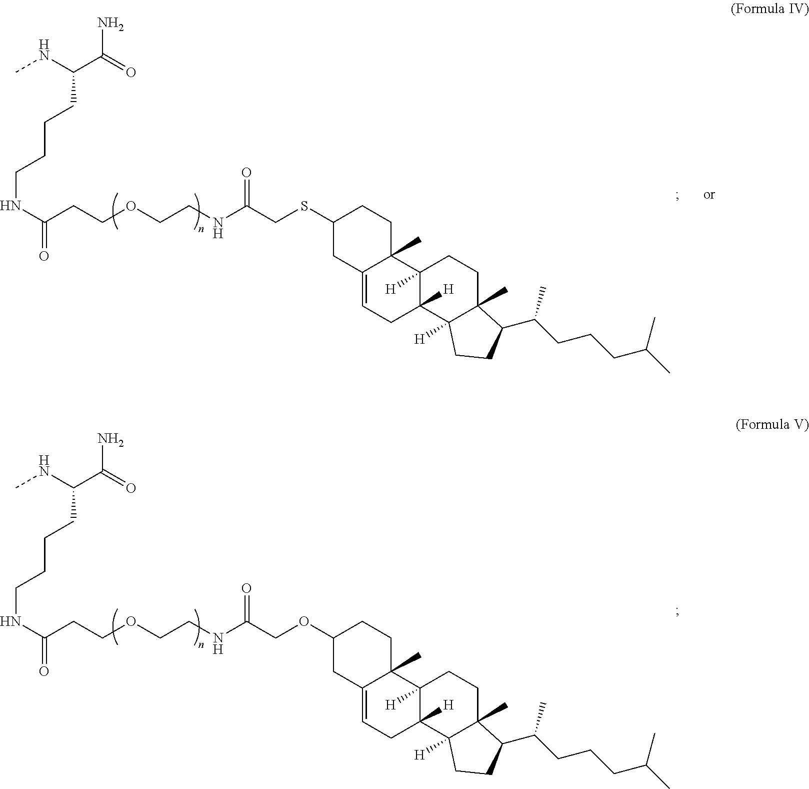

wherein n is 1-36, optionally wherein n is 16 or 20.

Also provided herein is a conjugate comprising or consisting of FD8SISQVNXKINQSLAFIRKSDELLHNV * (SEQ ID NO:261), wherein 8 is internally cross-linked to X, wherein 8 is (R)-α-(7′-octenyl)alanine, wherein X is (S)-α-(4′-pentenyl)alanine, wherein * is the formula

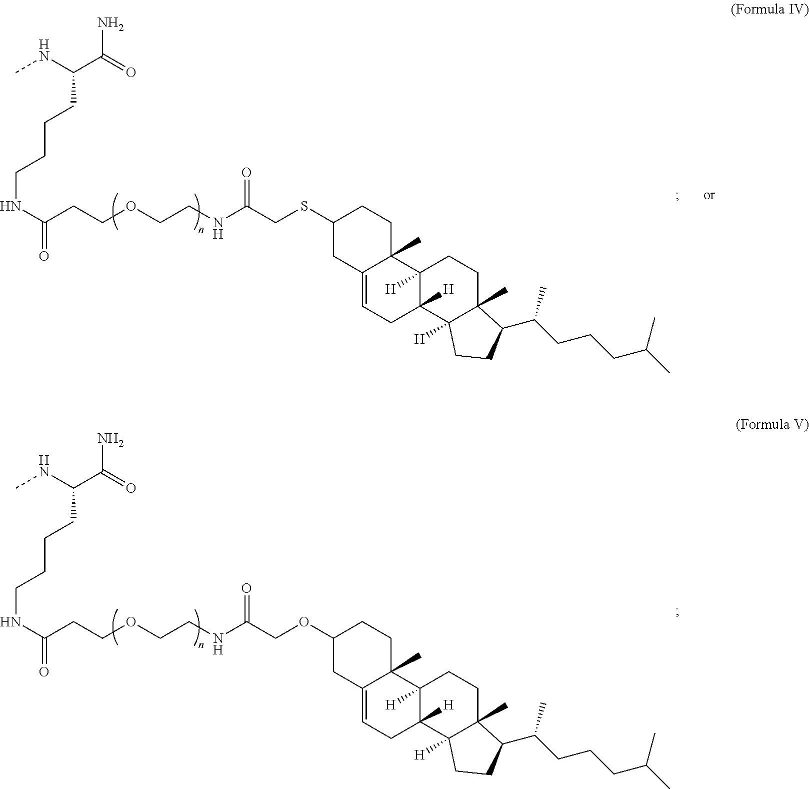

wherein n is 1-36, optionally wherein n is 16 or 20.

Also provided herein is a conjugate comprising or consisting of FDASISQVNEKINQSL8FIRKSDXLLHNV* (SEQ ID NO:266), wherein 8 is internally cross-linked to X, wherein 8 is (R)-α-(7′-octenyl)alanine, wherein X is (S)-α-(4′-pentenyl)alanine, wherein * is the formula

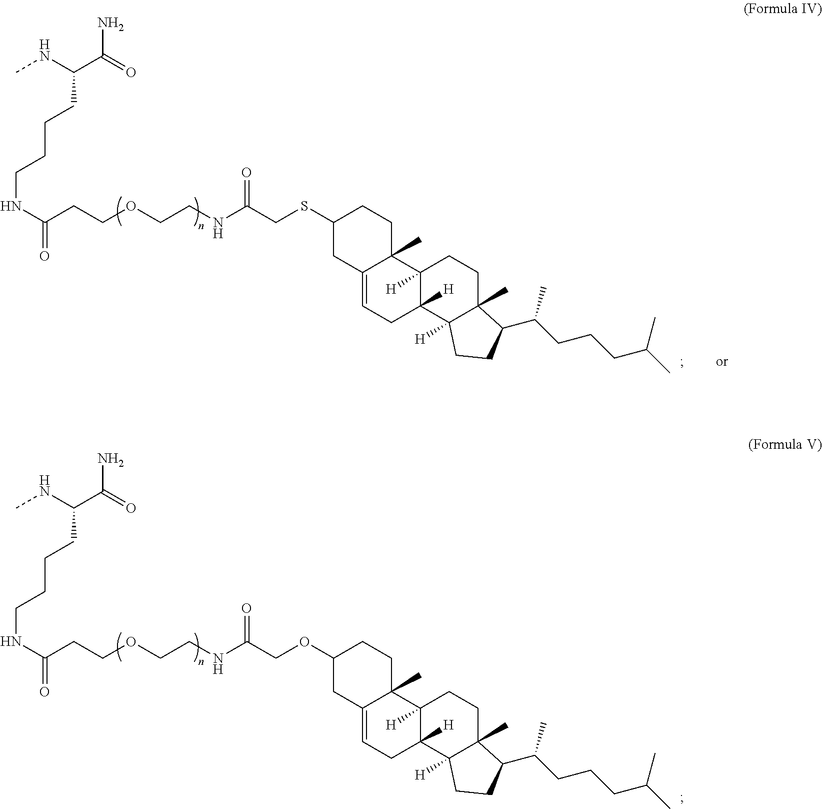

wherein n is 1-36, optionally wherein n is 16 or 20.

Also provided herein is a structurally-stabilized peptide, comprising: an internally cross-linked amino acid sequence comprising 25 to 29 contiguous amino acids of the sequence FDASISQVNEKINQSLAFIRKSDELLHNV (SEQ ID NO:100), except for two to six amino acid substitutions relative to the sequence of SEQ ID NO:100; wherein two of the two to six amino acid substitutions are with α, α-disubstituted non-natural amino acids with olefinic side chains cross-linked to each other, wherein the α, α-disubstituted non-natural amino acids with olefinic side chains cross-linked to each other are separated by three or six amino acids; wherein the structurally-stabilized peptide binds to an RSV 5-helix bundle protein and/or wherein the structurally-stabilized peptide inhibits infection of a cell by RSV and/or prevents infection of a cell by RSV; and wherein the structurally-stabilized peptide is 25 to 30 amino acids in length, optionally wherein the structurally-stabilized peptide is 29 amino acids in length. In some instances, the internally cross-linked amino acid sequence further comprises or consists of the sequence X1X2X3 wherein X1 is any amino acid, optionally S; X2 and X3 are any negatively charged amino acid immediately upstream of the first amino acid of SEQ ID NO:100 (i.e., immediately upstream of the first F of SEQ ID NO:100). In some cases X1X2X3=SDE. In one instance, the amino acid sequence comprises or consists of SDEFDASISQVNEKINQSLAFIRKSDELLHNV (SEQ ID NO:280) except for two to six amino acid substitutions relative to the sequence of SEQ ID NO: 100 and wherein the internally cross-linked amino acid sequence does not include the sequence NAGKST (SEQ ID NO:258); wherein two of the two to six amino acid substitutions are with α, α-disubstituted non-natural amino acids with olefinic side chains cross-linked to each other, wherein the α, α-disubstituted non-natural amino acids with olefinic side chains cross-linked to each other are separated by three or six amino acids; wherein the conjugate binds to an RSV 5-helix bundle protein and/or wherein the conjugate inhibits infection of a cell by RSV and/or prevents infection of a cell by RSV; and wherein the conjugate is 25 to 45 amino acids in length, optionally wherein the conjugate is 30 amino acids in length.

Also provided herein is a structurally-stabilized peptide, comprising: an internally cross-linked amino acid sequence having the formula:

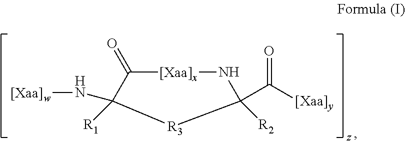

or a pharmaceutically acceptable salt thereof;

wherein each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted; wherein x is 3 or 6; wherein each R₃ is independently alkylene, alkenylene, or alkynylene, any of which is substituted or unsubstituted; wherein z is 1, 2, 3, 4, 5, 6, 7, 8, 9, or 10; and wherein the internally cross-linked amino sequence comprises 25 to 29 contiguous amino acids of the sequence of SEQ ID NO:100, except for two to six amino acid substitutions relative to the sequence of SEQ ID NO:100; wherein the structurally-stabilized peptide binds to an RSV 5-helix bundle protein and/or wherein the structurally-stabilized peptide inhibits infection of a cell by RSV and/or prevents infection of a cell by RSV; and wherein the structurally-stabilized peptide is 25 to 30 amino acids in length, optionally wherein the structurally-stabilized peptide is 29 amino acids in length.

In some instances of the foregoing structurally-stabilized peptide, the structurally-stabilized peptide does not comprise the amino acids corresponding to positions 517-522, 517-521, 517-520, 517-519, or 517-518 of an RSV-F protein (numbered according to the sequence set forth in SEQ ID NO:1). In some instances, the internally cross-linked amino acid sequence does not include the sequence NAGKST (SEQ ID NO:258).

In some instances of the foregoing structurally stabilized peptide, the structurally-stabilized peptide is 29 amino acids in length and comprises 29 contiguous amino acids of the sequence of SEQ ID NO:100, except for two to six amino acid substitutions relative to the sequence of SEQ ID NO:100. In some instances of the foregoing structurally stabilized peptide, the structurally-stabilized peptide is 32 amino acids in length and comprises 32 contiguous amino acids of the sequence of SEQ ID NO:280, except for two to six amino acid substitutions relative to the sequence of SEQ ID NO:280.

In some instances of the foregoing structurally-stabilized peptide, the structurally-stabilized peptide comprises the amino acid sequence set forth in any one of SEQ ID NOs: 27-70. In some instances, the structurally-stabilized peptide comprises the amino acid sequence set forth in any one of SEQ ID NOs: 27, 29, 36, 43, and 46. In some instances, the structurally-stabilized peptide comprises the amino acid sequence set forth in any one of SEQ ID NOs: 49, 51, 58, 65, and 68.

Also provided herein is a peptide, comprising the amino acid sequence of any one of SEQ ID NOs:27-70, except for zero to six additional substitutions, wherein the peptide does not include the sequence NAGKST (SEQ ID NO:258).

Also provided herein is a pharmaceutical composition comprising any one of the foregoing conjugates, any one of the foregoing structurally-stabilized peptides, or any one of the foregoing peptides, and a pharmaceutically acceptable carrier.

Also provided herein is a method of treating an RSV infection in a subject in need thereof, the method comprising administering to the subject a therapeutically-effective amount of any one of the foregoing conjugates, any one of the foregoing structurally-stabilized peptides, or any one of the foregoing peptides. In some instances, the subject is a human.

Also provided herein is a method of preventing an RSV infection in a subject in need thereof, the method comprising administering to the subject a therapeutically-effective amount of any one of the foregoing conjugates, any one of the foregoing structurally-stabilized peptides, or any one of the foregoing peptides. In some instances, the subject is a human.

Also provided herein is a method of making a structurally-stabilized peptide, the method comprising: (a) providing a peptide having an amino acid sequence comprising 25 to 29 contiguous amino acids of the sequence FDASISQVNEKINQSLAFIRKSDELLHNV (SEQ ID NO:100), except for two to six amino acid substitutions relative to the sequence of SEQ ID NO:100; wherein two of the two to six amino acid substitutions are with α, α-disubstituted non-natural amino acids with olefinic side chains, wherein the α, α-disubstituted non-natural amino acids with olefinic side chains are separated by three or six amino acids; and (b) cross-linking the peptide, thereby making the structurally-stabilized peptide, and optionally purifying the structurally-stabilized peptide. In some instances, the cross-linking is by a ruthenium catalyzed metathesis reaction. In some instances, the method further comprises derivatizing a resin bound amine of the structurally-stabilized peptide with PEG and/or cholesterol or thiocholesterol containing a carboxylic acid on a resin. In some instances, the method further comprises formulating the structurally-stabilized peptide as a sterile pharmaceutical composition.

Also provided herein is a pharmaceutical composition comprising (a) a means for treating or preventing an RSV infection in a subject, and (b) a pharmaceutically acceptable carrier; optionally wherein the subject is a human. In some instances, the means for treating or preventing an RSV infection are structurally-stabilized RSV peptides or cholesterol- or thiocholesterol-conjugates thereof.

In another aspect the disclosure features a peptide comprising or consisting of the amino acid sequence X1X2X3FDASISQVNEKINQSLAFIRKSDELLHNV (SEQ ID NO: 282) with 0, 1, 2, or 3 amino acid substitutions (optionally wherein the substitutions include positions 20 and 27 of SEQ ID NO:282), wherein X1 is any amino acid, optionally S, and X2 and X3 are negatively charged amino acids. In one case, the peptide comprises or consists of the amino acid sequence SDEFDASISQVNEKINQSLAFIRKSDELLHNV (SEQ ID NO:280) with 0, 1, 2, or 3 amino acid substitutions (optionally wherein the substitutions include positions 20 and 27 of SEQ ID NO:280).

In another aspect, the disclosure relates to an internally cross-linked peptide comprising or consisting of the sequence XIX2X3FDASISQVNEKINQSL8FIRKSDXLLHNV (SEQ ID NO:284), wherein X1 is any amino acid, optionally S; X2 and X3 are negatively charged amino acids; 8″=(R)-a-(7′-octenyl)alanine; and “X”=(S)-α-(4′-pentenyl)alanine. In one case, the peptide is an internally cross-linked peptide that comprises or consists of the amino acid sequence SDEFDASISQVNEKINQSL8FIRKSDXLLHNV (SEQ ID NO: 285), wherein “8″=(R)-α-(7′-octenyl)alanine; and “X”=(S)-α-(4′-pentenyl)alanine.

In yet another aspect, the disclosure features a conjugate comprising the amino acid sequence X1X2X3FDASISQVNEKINQSL8FIRKSDXLLHNV* (SEQ ID NO:286), wherein X1 is any amino acid, optionally S; X2 and X3 are negatively charged amino acids; 8″=(R)-α-(7′-octenyl)alanine; “X”=(S)-α-(4′-pentenyl)alanine; and * =linker-lipid. In one instance, the * comprises Z-PEG(n)-cholesterol or Z-PEG(n)-thiocholesterol, wherein n=1-36, and wherein in Z is a diamino acid (e.g., lysine or ornithine. In one case, the conjugate comprises or consists of the amino acid sequence SDEFDASISQVNEKINQSL8FIRKSDXLLHNV* (SEQ ID NO: 281), wherein “8″=(R)-α-(7′-octenyl)alanine; “X”=(S)-α-(4′-pentenyl)alanine; and * =linker-lipid. In one instance, the * comprises Z-PEG(n)-cholesterol or Z-PEG(n)-thiocholesterol, wherein n=1-36, and wherein in Z is a diamino acid (e.g., lysine or ornithine.

In some cases, the above peptides, internally cross-linked peptides, or conjugates are useful in methods of treating or preventing n RSV infection in a subject in need thereof, the method comprising administering to the subject a therapeutically-effective amount of any one of the foregoing peptides, internally cross-linked peptides, or conjugates. In some instances, the subject is a human.

The disclosure also features pharmaceutical compositions comprising any one of the foregoing peptides, internally cross-linked peptides, or conjugate, and a pharmaceutically acceptable carrier.

### BRIEF DESCRIPTION OF DRAWINGS

FIG. 1 depicts a mechanism of action of RSV viral-host membrane fusion (top) and RSV-F stapled lipopeptide inhibition of membrane fusion and viral infection (bottom).

FIG. 2 depicts the amino acid sequence of the F protein (SEQ ID NO: 1) of RSV. Heptad repeat domain 1 (HR1) (SEQ ID NO:2) is in bold. Heptad repeat domain 2 (HR2) (SEQ ID NO:3) is underlined.

FIG. 3 is a schematic representation of the RSV glycoprotein (F) protein, including the sequence of the HR1 (SEQ ID NO: 2) and HR2 (SEQ ID NO: 3) fusion domains.

FIG. 4 depicts an alignment of the HR1 (top) and HR2 (bottom) regions of various RSV virus strains. HR1 sequences, from top to bottom: SEQ ID NOs:2, 2, 93, 94, and 95, respectively; HR2 sequences, from top to bottom: SEQ ID NOs:3, 3, 96, 97, and 97, respectively. For each of HR1 and HR2, the bottom sequences (SEQ ID NOs:98 and 99, respectively, represent conserved amino acids.

FIG. 5 depicts a variety of stapling amino acids containing olefinic tethers that can be used to generate hydrocarbon stapled RSV-F HR2 peptides bearing staples spanning i, i+3; i, i+4; and i, i+7 positions. Top row, from left to right: (R)-α-(7′-octenyl)alanine, (S)-α-(7′-octenyl)alanine, (R)-α-(4′-pentenyl)alanine, (S)-α-(4′-pentenyl)alanine, (R)-α-(2′-propenyl)alanine, and bis-pentenyl glycine, respectively.

FIG. 6 depicts a variety of staple compositions in multiply stapled peptides and staple scanning to generate a library of multiply stapled RSV-F HR2 peptides for conjugation to cholesterol or cholesterol variant moieties (e.g., PEG-thiocholesterol or PEG-cholesterol).

FIG. 7 depicts a variety of staple compositions in tandem stitched peptides to generate a library of stitched RSV-F HR2 peptides for conjugation to cholesterol or cholesterol variant moieties (e.g., PEG-thiocholesterol or PEG-cholesterol).

FIG. 8 is an illustration of an exemplary approach to designing, synthesizing, and identifying optimal stapled peptide constructs to target the RSV-F fusion apparatus, including the generation of Ala scan, staple scan, and variable N- and C-terminal deletion, addition, and derivatization libraries for conjugation to cholesterol or cholesterol variant moieties (e.g., PEG-thiocholesterol or PEG-cholesterol). Singly and doubly stapled and stitched constructs, including alanine and staple and stitch scans, are used to identify optimal stapled peptides for conjugation to cholesterol or cholesterol variant moieties (e.g., PEG-thiocholesterol or PEG-cholesterol moieties of variable PEG chain length), and application in in vitro and in vivo analyses.

FIG. 9 depicts a dose response curve of a single stapled peptide (SEQ ID NO: 4; 8 is (R)-α-(7′-octenyl)alanine) and X is (S)-α-(4′-pentenyl)alanine) upon treatment of A549 cells infected by a GFP-RSV virus.

FIG. 10 depicts the crystal structure of the RSV six helix bundle (RCSB PDB: 1G2C). The first and last amino acids of the extended helix (corresponding to Phe-488 and Val-516 of the sequence of SEQ ID NO:1) are denoted.

FIG. 11 depicts a synthetic schema for converting thiocholesterol or cholesterol into a carboxylic acid for facile on-resin derivatization of stapled peptides with cholesterol- (or cholesterol variant-, e.g., thiocholesterol) containing moieties. DCM: dichloromethane; TFA: trifluoroacetic acid; eq: equivalents; RT: room temperature; min: minutes; hr: hours; vol: volume; A: heat.

FIG. 12 depicts a synthetic schema of the steps for on-resin derivatization of a stapled peptide sequence (exemplified with the sequence of SEQ ID NO: 5) with a PEG-linked thiocholesterol moiety. DMF: Dimethylformamide; HATU: 1-[Bis(dimethylamino)methylene]-1H-1,2,3-triazolo[4,5-b]pyridinium 3-oxide hexafluorophosphate; DIEA: N,N-Diisopropylethylamine. Figure discloses SEQ ID NOS 262-264, respectively, in order of appearance.

FIG. 13 depicts a dose response curve of a peptide having the sequence of SEQ ID NO:5 with a C-terminal (PEG)4 linker and thiocholesterol (“PEG4-TC”) appended. Dashed line at 50 represents vehicle infection level.

FIG. 14 depicts exemplary structurally-stabilized RSV-HR2 peptide sequences bearing C-terminal derivatization with * =Lys(PEG4-thiocholesterol; 8 is (R)-a-(7′-octenyl)alanine) and X is (S)-α-(4′-pentenyl)alanine) (SEQ ID NOs: 5-26).

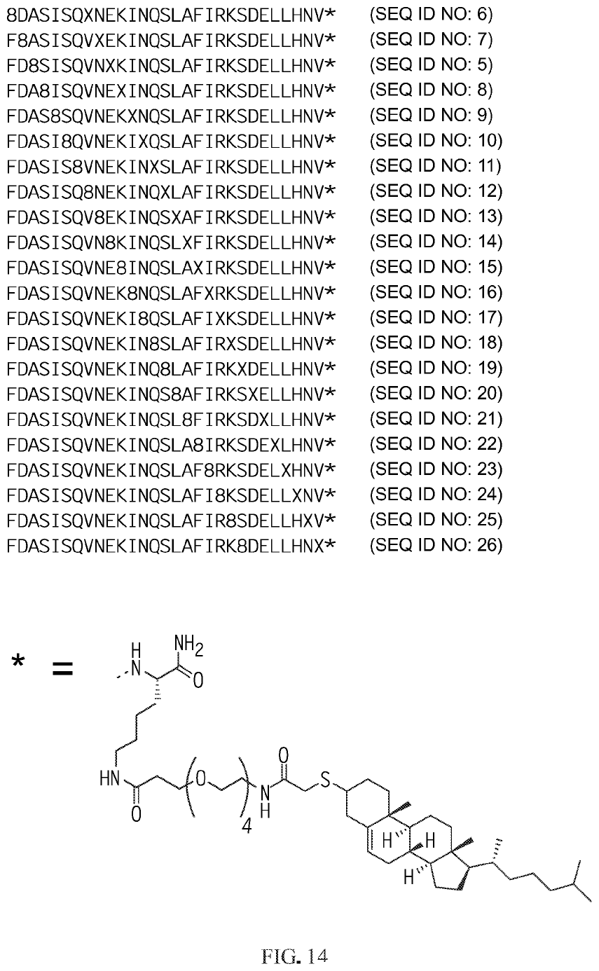

FIG. 15 is a graph depicting the differential antiviral activity of stapled peptides having the depicted sequences (* =Lys(PEG4-thiocholesterol; 8 is (R)-α-(7′-octenyl)alanine) and X is (S)-α-(4′-pentenyl)alanine), at a dose of 2 μM, against RSV-GFP in A549 cells after 48 hours.

FIG. 16 is a graph depicting a dose response curve of a stapled peptide having the amino acid sequence of SEQ ID NO:51 with an appended PEG(n)-thiocholesterol (“TC”) linked to the stapled peptide via a C-terminal lysine, wherein n is 0, 4, 8, 12, 16 or 20 (SEQ ID NOS 267-272, from top to bottom, respectively).

FIG. 17 is a graph depicting a dose response curve of a stapled peptide having the amino acid sequence of SEQ ID NO:65 with an appended PEG(n)-thiocholesterol linked to the stapled peptide via a C-terminal lysine, wherein n is 0, 4, 8, 12, 16 or 20 (SEQ ID NOS 273-278, from top to bottom, respectively).

FIG. 18 documents the solubility of two peptides SEQ ID NO: 21 (visible pellet) and SEQ ID NO: 281 (fully soluble). Buffer conditions are 15 mg/mL in 15% DMSO, PBS pH 7.4.

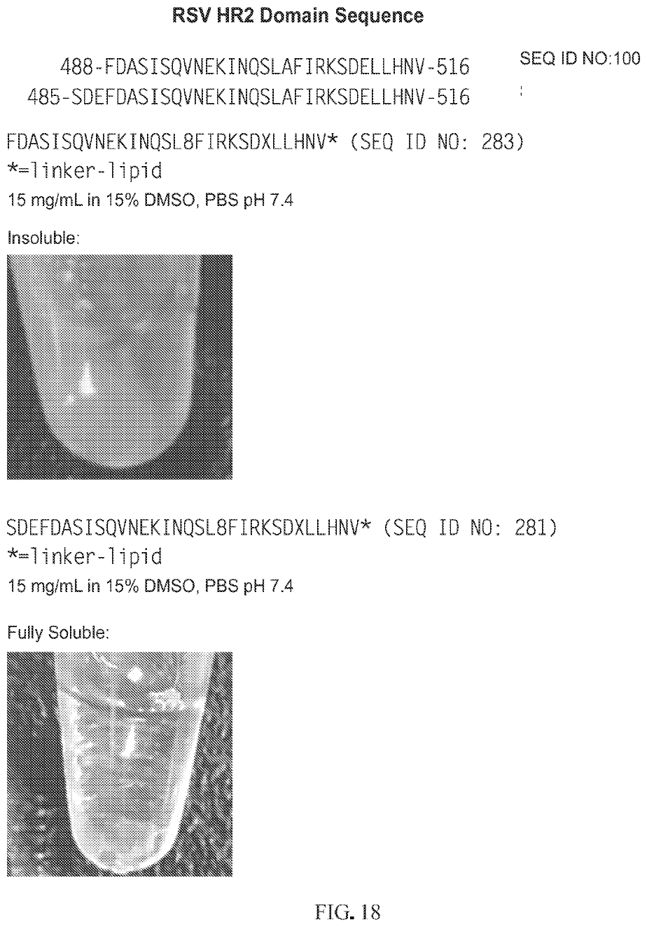

### DETAILED DESCRIPTION

The present disclosure is based, inter alia, on structurally-stabilized (e.g., stapled, e.g., hydrocarbon stapled) RSV peptides and the discovery that they may be lipidated (e.g., with PEG and/or cholesterol (or a variant of cholesterol, e.g., thiocholesterol), e.g., PEG(n)-cholesterol or PEG(n)-thiocholesterol) to selectively bind to RSV and exhibit antiviral activity against RSV. Accordingly, the present disclosure provides methods (e.g., approaches to convert cholesterol/thiocholesterol into carboxylic acids for on-resin derivatization) and compositions (e.g., structurally-stabilized RSV peptides and PEG(n)-cholesterol or PEG(n)-thiocholesterol conjugates) for treating, for developing treatments for, and for preventing infection or disease with an RSV. Thus, the peptides and compositions disclosed herein can be used to prevent and/or treat an RSV infection.

### RSV Peptides

RSV deploys discrete heptad repeat domains of its F protein to form the RSV-SFB, whose structure enables the virus to penetrate the host cell membrane. Furin-like protease cleaves the RSV-F precursor, resulting in the formation of two subunits stabilized by a disulfide bridge (Gonzalez-Reyes et al., Proc. Natl. Acad. Sci., USA, 98:9859-9864, 2003; Sugrue et al., Gen. Virol., 82:1375-1386, 2001). This cleavage also unveils an otherwise hidden peptidic fusion motif located at the N-terminus of the F1 subunit leading to formation of the FSV-F lollipop structure (Matthews et al., J. Virol., 74:5911-5920, 2000; Smith et al., Protein Eng., 15:365-371, 2002). Once the fusion peptide inserts into the host cell membrane, the F protein refolds itself to form a trimeric hairpin or “6-helix bundle.” The trimeric hairpin derives from a poorly characterized conformational change that brings the C-terminal heptad repeat (HR2) region into anti-parallel assembly with the N-terminal heptad repeat (HR1), which is juxtaposed to the fusion peptide (Cianci et al., Proc. Natl. Acad. Sci., USA, 101:15046-15051, 2004). After pore opening, the HR-induced conformational change achieves a new equilibrium state believed to be essential to stabilizing and enlarging the pore (Cianci, supra, Melikyan, Retrovirology, 5:111, 2008; Melikyan et al., Proc. Natl. Acad. Sci., USA, 102:8728-8733, 2005), and penetrating the host cell. The compositions and methods disclosed herein can be used to prevent or treat RSV infection by inhibiting this process.

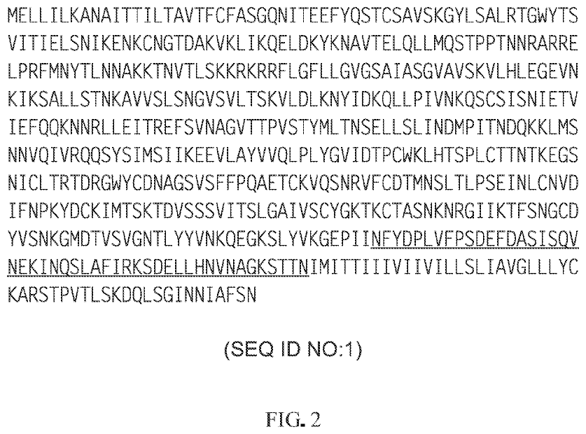

Provided herein are RSV-F HR2 peptides. RSV infection is mediated at the cell surface by the RSV-F protein, which has two heptad repeat domains: HR1 and HR2. The amino acid sequence (SEQ ID NO:1) of an exemplary RSV-F protein sequence is depicted in FIG. 2. The amino acid sequence of an exemplary RSV-F protein HR1 is set forth in SEQ ID NO:2. The amino acid sequence of an exemplary RSV-F protein HR2 is set forth in SEQ ID NO:3.

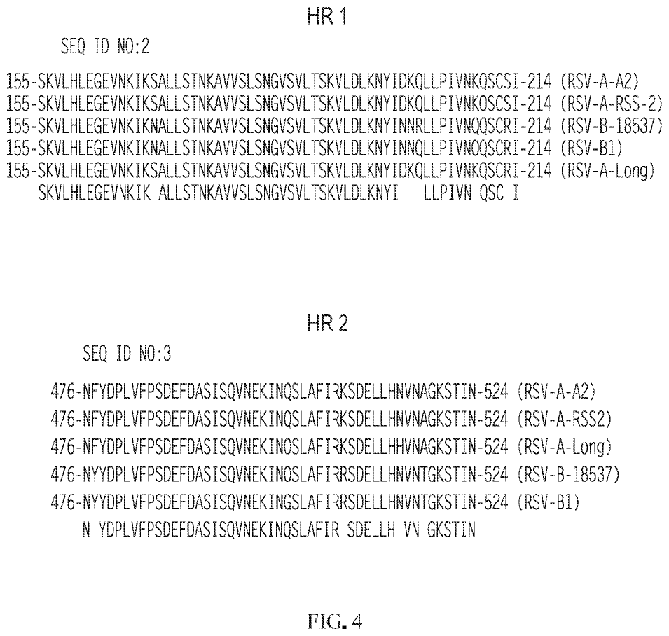

The RSV-F protein HR1 and HR2 among the different RSV strains have high homology. See FIG. 4 for an alignment of exemplary amino acid sequences for HR1 and HR2 sequences of exemplary RSV strains.

In some instances, an HR2 peptide described herein comprises or consists of the amino acid sequence of SEQ ID NO:100. In some instances, the HR2 peptide consists of the amino acid sequence of SEQ ID NO:100. In some instances, the HR2 peptide does not comprise the amino acid sequence NAGKST (SEQ ID NO:258). In some instances, the HR2 peptide does not comprise the amino acid sequence NAGKS (SEQ ID NO:259). In some instances, the HR2 peptide does not comprise the amino acid sequence NAGK (SEQ ID NO:260). In some instances, the HR2 peptide does not comprise the amino acid sequence NAG. In some instances, the HR2 peptide does not comprise the amino acid sequence NA. In some instances, the HR2 peptide does not comprise the amino acids corresponding to positions 517-522, 517-521, 517-520, 517-519, or 517-518 of SEQ ID NO:1 (or the corresponding amino acids of an RSV-F protein of another strain (see, e.g., FIG. 4)). In some instances, the HR2 peptide is 25 to 34 (e.g., 25, 26, 27, 28, 29, 30, 31, 32, 33, or 34) amino acids in length. In some instances, the HR2 peptide is 30 amino acids in length. In certain instances, the HR2 peptide further comprises or consists of the sequence X1X2X3 wherein X1 is any amino acid, optionally S; X2 and X3 are any negatively charged amino acid immediately upstream of the first amino acid of SEQ ID NO: 100 (i.e., immediately upstream of the first F of SEQ ID NO:100). In some cases X1X2X3=SDE. In one instance, the amino acid sequence comprises or consists of SDEFDASISQVNEKINQSLAFIRKSDELLHNV (SEQ ID NO:280).

In certain instances, an RSV-F HR2 peptide described herein comprises or consists of the amino acid sequence of SEQ ID NO: 100 or 280, except that it contains one or more (e.g., 1, 2, 3, 4, 5, 6, 7, 8, 9, 10) amino acid substitutions (e.g., relative to the amino acid sequence of SEQ ID NO: 100 or 280), e.g., one or more (e.g., 1, 2, 3, 4, 5, 6, 7, 8, 9, 10) conservative and/or non-conservative amino acid substitutions.

A “conservative amino acid substitution” means that the substitution replaces one amino acid with another amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), aromatic side chains (e.g., tyrosine, phenylalanine, tryptophan, histidine), and acidic side chains and their amides (e.g., aspartic acid, glutamic acid, asparagine, glutamine).

The skilled artisan will appreciate that an alignment of the F proteins of different RSV strains is useful in identifying conserved residues and residues amenable to substitution (e.g., conservative or non-conservative substitution). For instance, a residue that is unchanged between two or more different RSV strains (see, e.g., FIG. 4) in such an alignment may be either unmodified or substituted with a non-natural amino acid or with a conservative amino acid substitution. A residue that differs by a conservative amino acid substitution in two or more different RSV strains (see, e.g., FIG. 4) in such an alignment may be either unmodified or substituted with a conservative amino acid substitution. A residue that is not conserved in two or more different RSV strains (see, e.g., FIG. 4) in such an alignment may be either unmodified or replaced by any amino acid. In some instances, residues that are conserved between two or more different RSV strains in such an alignment but which are located on the non-interacting face of HR2 can be replaced by any amino acid. For example, in view of the alignment in FIG. 4, a conservative amino acid substitution is permitted at the amino acids corresponding to position 33 of SEQ ID NO:3 and a non-conservative amino acid substitution is permitted at position 40 of SEQ ID NO:3. In certain instances, the substituted amino acid(s) are selected from the group consisting of L-Ala, D-Ala, Aib, Sar, Ser, a substituted alanine, or a substituted glycine derivative. Methods for identifying the interactive face of a peptide are known in the art (see, e.g., Broglia et al., Protein sci., 14(10):2668-81, 2005; Hammond et al., J. Pharm. Sci., 98(1):4589-603, 2009; Ng and Yang, J. Phys. Chem. B., 111(50):13886-93, 2007; and Bird et al., PNAS USA, 197:14093, 2010).

In some instances, a peptide described herein comprises an amino acid sequence that is at least at least 70%, at least 75%, at least 80%, at least 85%, at least 90%, or at least 93% identical to sequence set forth in SEQ ID NO:100 or 280. In some instances, a peptide as described above has one or more (e.g., 1, 2, 3, 4, 5) of the following properties: (i) are alpha-helical; (ii) are protease resistant; (iii) bind to an RSV 5-helix bundle protein; (iv) inhibits infection of a cell by RSV; and/or (v) prevents infection of a cell by RSV. In some instances, the peptides inhibit infection of a cell by RSV in pseudovirus and/or live RSV virus assays and/or prevent infection of a cell by RSV in the pseudovirus and/or the live RSV virus assays. RSV pseudovirus assays are known in the art, see, e.g., Haid et al., 2015. J Virol 90:3065-3073, which is incorporated by reference herein in its entirety.

Methods for determining percent identity between amino acid sequences are known in the art. For example, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The determination of percent identity between two amino acid sequences is accomplished using the BLAST 2.0 program. Sequence comparison is performed using an ungapped alignment and using the default parameters (Blossom 62 matrix, gap existence cost of 11, per residue gapped cost of 1, and a lambda ratio of 0.85). The mathematical algorithm used in BLAST programs is described in Altschul et al. (Nucleic Acids Res. 25:3389-3402, 1997).

In some instances, an RSV-F HR2 peptide described herein (e.g., SEQ ID NO: 100, 280) contains at least one, at least 2, at least 3, at least 4, or at least 5 (e.g., 1, 2, 3, 4, 5, 6) amino acids added to the N-terminus of the peptide. In some instances, an RSV-F HR2 peptide described herein (e.g., SEQ ID NO: 100, 280) contains at least one, at least 2, at least 3, at least 4, or at least 5 (e.g., 1, 2, 3, 4, 5, 6) amino acids added to the C-terminus of the peptide. In some instances, an RSV-F HR2 peptide described herein (e.g., SEQ ID NO: 100, 280) contains at least one, at least 2, at least 3, at least 4, or at least 5 amino acids (e.g., 1, 2, 3, 4, 5, 6) deleted at the N-terminus of the peptide. In some instances, an RSV-F HR2 peptide described herein (e.g., SEQ ID NO: 100) contains at least one, at least 2, at least 3, at least 4, or at least 5 amino acids (e.g., 1, 2, 3, 4, 5, 6) deleted at the C-terminus of the peptide. In some instances, the RSV-F 1R₂ peptide does not comprise the amino acid sequence NAGKST (SEQ ID NO:258).

In some instances, the peptide includes an amino acid sequence that has 2 to 10, 2 to 9, 2 to 8, 2 to 7, 2 to 6, 2 to 5, 2 to 4, 2 to 3, or 2 substitutions, insertions, and/or deletions relative to SEQ ID NO: 100. In some instances, the peptides include 2, 3, 4, 5, or 6 substitutions, insertions, and/or deletions relative to SEQ ID NO: 100 or 280. In some instances, a peptide having substitutions, insertions, and/or deletions relative to SEQ ID NO: 100 or 280 as described above has one or more (e.g., 1, 2, 3, 4, 5) of the properties listed below: (i) are alpha-helical; (ii) are protease resistant; (iii) bind to an RSV 5-helix bundle protein; (iv) inhibits infection of a cell by RSV; and/or (v) prevents infection of a cell by RSV. In some instances, the peptides inhibit infection of a cell by RSV in pseudovirus and/or live RSV virus assays and/or prevent infection of a cell by RSV in the pseudovirus and/or the live RSV virus assays.

In some instances, the peptide is 25 to 45 (e.g., 25, 26, 27, 28, 29, 30, 31, 32, 33, 34, 35, 36, 37, 38, 39, 40, 41, 42, 43, 44, 45) amino acids in length. In some instances, the peptide is 28 to 40 (e.g., 25, 26, 27, 28, 29, 30, 31, 32, 33, 34, 35, 36, 37, 38, 39, 40) amino acids in length. In some instances, the peptide is 25 to 35 (e.g., 25, 26, 27, 28, 29, 30, 31, 32, 33, 34, 35) amino acids in length. In some instances, the peptide is 29 or 30 amino acids in length. In certain instances, the peptide is 32 or 33 amino acids in length.

In some instances, the peptides described above have one or more (e.g., 1, 2, 3, 4, 5) of the following properties: (i) are alpha-helical; (ii) are protease resistant; (iii) bind to an RSV 5-helix bundle protein; (iv) inhibits infection of a cell by RSV; and/or (v) prevents infection of a cell by RSV. In some instances, the peptides inhibit infection of a cell by RSV in pseudovirus and/or live RSV virus assays and/or prevent infection of a cell by RSV in the pseudovirus and/or the live RSV virus assays.

In certain instances, each of the RSV-F HR2 peptides described above bind to an RSV 5-helix bundle protein. In certain instances, each of the RSV-F HR2 peptides described above binds to an RSV 5-helix bundle protein and prevents or blocks fusion of an RSV membrane and a host membrane.

Methods of determining whether a peptide (e.g., an RSV-F HR2 peptide, a structurally-stabilized peptide, a structurally-stabilized peptide conjugate described herein) binds to an RSV 5-helix bundle protein are known in the art, such as, high resolution clear native electrophoresis (hrCNE). See, e.g., Example 3 of WO 2013/102211, which is incorporated by reference herein in its entirety.

Methods of determining whether a peptide (e.g., an RSV-F HR2 peptide, a structurally-stabilized peptide or a structurally-stabilized peptide conjugate described herein) prevents or blocks fusion of an RSV membrane and a host membrane are known in the art, such as, e.g., cytotoxicity and immunofluorescence. In some instances, a peptide prevents or blocks fusion of an RSV membrane and a host membrane if less than 1%, less than 5%, less than 10%, less than 15% less than 20%, less than 30%, less than 40%, or less than 50% of cells are infected with RSV or an RSV pseudovirus at a multiplicity of infection of 0.1, 0.5, 1, or 10 in the presence the peptide. In some instances, a peptide prevents or blocks fusion of an RSV membrane and a host membrane if less than 1%, less than 5%, less than 10%, less than 15% less than 20%, less than 30%, less than 40%, or less than 50% of cells exhibit fusion of the RSV membrane and the host membrane after infection with RSV at a multiplicity of infection of 0.1, 0.5, 1, or 10 in the presence the peptide.

Methods of determining whether a peptide (e.g., an RSV-F HR2 peptide, a structurally-stabilized peptide, a structurally-stabilized peptide conjugate described herein) inhibits infection of a cell by RSV are known in the art, such as, e.g., cytotoxicity and immunofluorescence, and described in the working examples. In some instances, a peptide (e.g., an RSV-F HR2 peptide, a structurally-stabilized peptide or a structurally-stabilized peptide conjugate described herein) inhibits infection of a cell by RSV if less than 1%, less than 5%, less than 10%, less than 15% less than 20%, less than 30%, less than 40%, or less than 50% of cells are infected with an RSV or an RSV pseudovirus at a multiplicity of infection of 0.1, 0.5, 1, or 10 in the presence the peptide. In some instances, a peptide (e.g., an RSV-F HR2 peptide, a structurally-stabilized peptide or a structurally-stabilized peptide conjugate described herein) inhibits infection of a cell if the level of RSV infection of a population of cells in the presence of the peptide is at least 20%, at least 30%, at least 40%, at least 50%, at least 60%, at least 70%, at least 80%, at least 90%, or at least 95% less than the level of RSV infection of a population of cells in the absence of the peptide under the same conditions. In some instances, the infection with the RSV is at a multiplicity of infection of 0.1, 0.5, 1, or 10.

In some instances at least two (e.g., 2, 3, 4, or 5) amino acids (e.g., separated by 3 or 6 amino acids) of an RSV-F HR2 peptide described herein (e.g., SEQ ID NO: 100 or a modified version thereof, e.g., described herein) are substituted with a, a-disubstituted non-natural amino acids, each with an olefinic side chain (e.g., a non-natural amino acid substituted with an alpha-methyl group and an alpha-alkenyl group), wherein the α, α-disubstituted non-natural amino acids can be cross-linked to each other to form one or more staples or stitches (see the section “Structurally-Stabilized Peptides” below). The type of substitutions that are made can, e.g., be guided by an alignment of the HR2 peptide of two or more RSV-F protein sequences (see, e.g., FIG. 4). The guidance provided in the “Structurally-Stabilized Peptides” section below regarding the amino acids that can be varied is equally relevant for the RSV-F HR2 peptides described herein.

In some cases, the RSV-F HR2 peptide (or a structurally-stabilized peptide) is lipidated. See the “Structurally-Stabilized Peptide Conjugates” section below. These lipidated peptides are interchangeably referred to herein as “structurally-stabilized peptide conjugates” and “conjugates”. In some cases, the RSV-F HR2 peptide is modified to comprise cholesterol, e.g., via a polyethylene glycol (PEG)-containing linker. In some cases, the RSV-F HR2 peptide is modified to comprise thiocholesterol, e.g., via a PEG-containing linker. In some cases, the RSV-F HR2 peptide (e.g., SEQ ID NO: 100, 280) includes the following formula affixed to the C-terminus of the peptide:

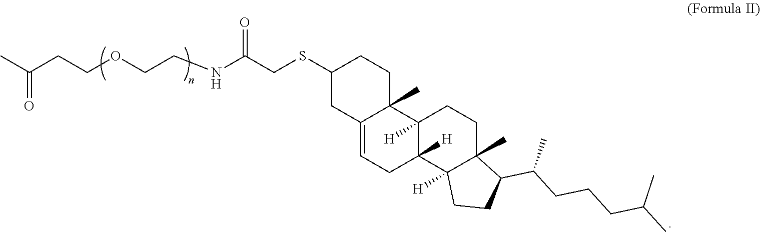

In some instance, the sulfur atom in the Formula II is replaced with an oxygen atom. In some cases, the RSV-F HR2 peptide (e.g., SEQ ID NO: 100, 280) includes the following formula affixed to the C-terminus of the peptide:

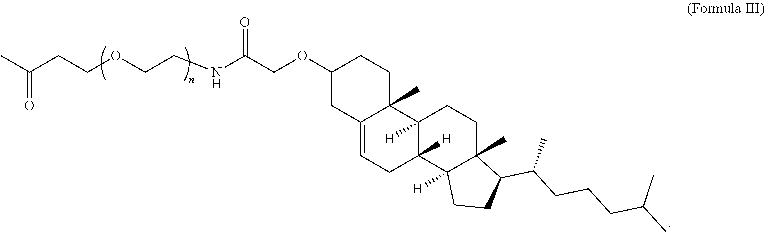

In some instances, n in the above Formula II or Formula II is n=1-36 (n=1, 2, 3, 4, 5, 6, 7,8,9, 10, 11, 12, 13, 14, 15, 16, 17, 18, 19, 20, 21, 22, 23, 24, 25, 26, 27, 28, 29, 30, 31, 32, 33, 34, 35, or 36). In some instances, n=16. In some instances, n=20.

### Structurally-Stabilized Peptides

Also provided herein are structurally-stabilized RSV-F HR2 peptides. In some instances, the structurally-stabilized peptide is a structurally-stabilized (e.g., stapled, e.g., hydrocarbon stapled) version of an RSV-F HR2 peptide described herein (see, e.g., the “RSV Peptides” section above). In some instances, the structurally-stabilized (e.g., stapled, e.g., hydrocarbon stapled) RSV-F HR2 peptides are derived from the sequence FDASISQVNEKINQSLAFIRKSDELLHNV (SEQ ID NO:100) or from SEQ ID NO:280.

In some instances, the structurally-stabilized peptide comprises an internally cross-linked amino acid sequence comprising 25 to 29 contiguous amino acids of the sequence FDASISQVNEKINQSLAFIRKSDELLHNV (SEQ ID NO: 100), except for two or more (e.g., 2, 3, 4, 5, 6, 7, 8, 9, 10) amino acid substitutions relative to the sequence of SEQ ID NO:100; wherein two of the two or more amino acid substitutions are with α, α-disubstituted non-natural amino acids with olefinic side chains cross-linked to each other, wherein the α, α-disubstituted non-natural amino acids with olefinic side chains cross-linked to each other are separated by six amino acids. In some instances, the structurally-stabilized peptide has 2 to 6 amino acid substitutions relative to the sequence of SEQ ID NO:100. In some instances, the structurally-stabilized peptide has 4 amino acid substitutions relative to the sequence of SEQ ID NO:100. In some instances, the structurally-stabilized peptide has 3 amino acid substitutions relative to the sequence of SEQ ID NO:100. In some instances, the structurally-stabilized peptide has 2 amino acid substitutions relative to the sequence of SEQ ID NO:100. In some instances, the substitutions with α, α-disubstituted non-natural amino acids with olefinic side chains cross-linked to each other are at positions (i) 1 and 8 (see, e.g., SEQ ID NO: 27), (ii) 3 and 10 (see, e.g., SEQ ID NO:29), (iii) 10 and 17 (see, e.g., SE QID NO:36), (iv) 17 and 24 (see, e.g., SEQ ID NO: 43), or (v) 20 and 27 (see, e.g., SEQ ID NO:46), numbered relative to SEQ ID NO:100 (wherein position 1 is the N-terminal phenylalanine of SEQ ID NO:100). In some instances, the substitutions with α, α-disubstituted non-natural amino acids with olefinic side chains cross-linked to each other are at positions 3 and 10 (see, e.g., SEQ ID NO:29), numbered relative to SEQ ID NO:100 (wherein position 1 is the N-terminal phenylalanine of SEQ ID NO:100). In some instances, the substitutions with α, α-disubstituted non-natural amino acids with olefinic side chains cross-linked to each other are at 17 and 24 (see, e.g., SEQ ID NO: 43), numbered relative to SEQ ID NO:100 (wherein position 1 is the N-terminal phenylalanine of SEQ ID NO:100). In some instances, the internally cross-linked amino acid sequence further comprises or consists of the sequence XIX2X3 wherein X1 is any amino acid, optionally S; X2 and X3 are any negatively charged amino acid immediately upstream of the first amino acid of SEQ ID NO:100 (i.e., immediately upstream of the first F of SEQ ID NO:100). In some cases X1X2X3=SDE. In one instance, the amino acid sequence comprises or consists of SDEFDASISQVNEKINQSLAFIRKSDELLHNV (SEQ ID NO:280) except for two to six amino acid substitutions relative to the sequence of SEQ ID NO:100 and wherein the internally cross-linked amino acid sequence does not include the sequence NAGKST (SEQ ID NO:258); wherein two of the two to six amino acid substitutions are with α, α-disubstituted non-natural amino acids with olefinic side chains cross-linked to each other, wherein the α, α-disubstituted non-natural amino acids with olefinic side chains cross-linked to each other are separated by three or six amino acids; wherein the conjugate binds to an RSV 5-helix bundle protein and/or wherein the conjugate inhibits infection of a cell by RSV and/or prevents infection of a cell by RSV; and wherein the conjugate is 25 to 45 amino acids in length, optionally wherein the conjugate is 30 amino acids in length.

In some instances, the structurally-stabilized peptide comprises an internally cross-linked amino acid sequence comprising 25 to 29 contiguous amino acids of the sequence FDASISQVNEKINQSLAFIRKSDELLHNV (SEQ ID NO:100), except for two or more (e.g., 2, 3, 4, 5, 6, 7, 8, 9, 10) amino acid substitutions relative to the sequence of SEQ ID NO: 100; wherein two of the two or more amino acid substitutions are with α, α-disubstituted non-natural amino acids with olefinic side chains cross-linked to each other, wherein the α, α-disubstituted non-natural amino acids with olefinic side chains cross-linked to each other are separated by three amino acids. In some instances, the structurally-stabilized peptide has 2 to 6 amino acid substitutions relative to the sequence of SEQ ID NO:100. In some instances, the structurally-stabilized peptide has 4 amino acid substitutions relative to the sequence of SEQ ID NO:100. In some instances, the structurally-stabilized peptide has 3 amino acid substitutions relative to the sequence of SEQ ID NO:100. In some instances, the structurally-stabilized peptide has 2 amino acid substitutions relative to the sequence of SEQ ID NO:100.

In some instances, the structurally-stabilized peptide comprises an internally cross-linked amino acid sequence comprising 30 to 32 contiguous amino acids of the sequence set forth in SEQ ID NO:280, except for two or more (e.g., 2, 3, 4, 5, 6, 7, 8, 9, 10) amino acid substitutions relative to the sequence of SEQ ID NO:280; wherein two of the two or more amino acid substitutions are with α, α-disubstituted non-natural amino acids with olefinic side chains cross-linked to each other, wherein the α, α-disubstituted non-natural amino acids with olefinic side chains cross-linked to each other are separated by three amino acids. In some instances, the structurally-stabilized peptide has 2 to 6 amino acid substitutions relative to the sequence of SEQ ID NO:280. In some instances, the structurally-stabilized peptide has 4 amino acid substitutions relative to the sequence of SEQ ID NO:280. In some instances, the structurally-stabilized peptide has 3 amino acid substitutions relative to the sequence of SEQ ID NO:280. In some instances, the structurally-stabilized peptide has 2 amino acid substitutions relative to the sequence of SEQ ID NO:280

In some instances, the structurally-stabilized peptide has one or more modifications (e.g., substitutions, insertions, additions, or deletions) described in the “RSV Peptides” section above.

In some instances, the structurally-stabilized (e.g., stapled, e.g., hydrocarbon stapled) peptide is a peptide shown in Table 1, below. In certain instances, this disclosure encompasses the structurally stabilized peptides of Table 1 further comprising three N-terminal amino acids X1X2X3, wherein X1 is any amino acid, optionally S; X2 and X3 are any negatively charged amino acid immediately upstream of the first amino acid of SEQ ID NO:100 (i.e., immediately upstream of the first F of SEQ ID NO:100). In some cases X1X2X3=SDE. In some instances, the structurally-stabilized peptide comprises or consists of the amino acid sequence of any one of SEQ ID NOs:27-70. In some instances, the structurally-stabilized peptide comprises or consists of the amino acid sequence of any one of SEQ ID NOs:27, 29, 36, 43, and 46. In some instances, the structurally-stabilized peptide comprises or consists of the amino acid sequence of any one of SEQ ID NOs: 49, 51, 58, 65, and 68. In some instances, the structurally-stabilized peptide comprises or consists of the amino acid sequence set forth in SEQ ID NO:29. In some instances, the structurally-stabilized peptide comprises or consists of the amino acid sequence set forth in SEQ ID NO:51. In some instances, the structurally-stabilized peptide comprises or consists of the amino acid sequence set forth in SEQ ID NO:43. In some instances, the structurally-stabilized peptide comprises or consists of the amino acid sequence set forth in SEQ ID NO:65. In some instances, the structurally-stabilized peptide comprises or consists of the amino acid sequence set forth in SEQ ID NO:281.

TABLE 1
Structurally-Stabilized RSV-F HR2 Peptides

| | SEQ ID |
|---|---|
| Sequence | NO |
| X₁DASISQX₂NEKINQSLAFIRKSDELLHNV | 27 |
| FX₁ASISQVX₂EKINQSLAFIRKSDELLHNV | 28 |
| FDX₁SISQVNX₂KINQSLAFIRKSDELLHNV | 29 |
| FDAX₁ISQVNEX₂INQSLAFIRKSDELLHNV | 30 |
| FDASX₁SQVNEKX₂NQSLAFIRKSDELLHNV | 31 |
| FDASIX₁QVNEKIX₂QSLAFIRKSDELLHNV | 32 |
| FDASISX₁VNEKINX₂SLAFIRKSDELLHNV | 33 |
| FDASISQX₁NEKINQX₂LAFIRKSDELLHNV | 34 |
| FDASISQVX₁EKINQSX₂AFIRKSDELLHNV | 35 |
| FDASISQVNX₁KINQSLX₂FIRKSDELLHNV | 36 |
| FDASISQVNEX₁INQSLAX₂IRKSDELLHNV | 37 |
| FDASISQVNEKX₁NQSLAFX₂RKSDELLHNV | 38 |
| FDASISQVNEKIX₁QSLAFIX₂KSDELLHNV | 39 |
| FDASISQVNEKINX₁SLAFIRX₂SDELLHNV | 40 |
| FDASISQVNEKINQX₁LAFIRKX₂DELLHNV | 41 |
| FDASISQVNEKINQSX₁AFIRKSX₂ELLHNV | 42 |
| FDASISQVNEKINQSLX₁FIRKSDX₂LLHNV | 43 |
| FDASISQVNEKINQSLAX₁IRKSDEX₂LHNV | 44 |
| FDASISQVNEKINQSLAFX₁RKSDELX₂HNV | 45 |
| FDASISQVNEKINQSLAFIX₁KSDELLX₂NV | 46 |
| FDASISQVNEKINQSLAFIRX₁SDELLHX₂V | 47 |
| FDASISQVNEKINQSLAFIRKX₁DELLHNX₂ | 48 |
| 8DASISQXNEKINQSLAFIRKSDELLHNV | 49 |
| F8ASISQVXEKINQSLAFIRKSDELLHNV | 50 |
| FD8SISQVNXKINQSLAFIRKSDELLHNV | 51 |
| FDA8ISQVNEXINQSLAFIRKSDELLHNV | 52 |
| FDAS8SQVNEKXNQSLAFIRKSDELLHNV | 53 |
| FDASI8QVNEKIXQSLAFIRKSDELLHNV | 54 |
| FDASIS8VNEKINXSLAFIRKSDELLHNV | 55 |
| FDASISQ8NEKINQXLAFIRKSDELLHNV | 56 |
| FDASISQV8EKINQSXAFIRKSDELLHNV | 57 |
| FDASISQVN8KINQSLXFIRKSDELLHNV | 58 |
| FDASISQVNE8INQSLAXIRKSDELLHNV | 59 |
| FDASISQVNEK8NQSLAFXRKSDELLHNV | 60 |
| FDASISQVNEKI8QSLAFIXKSDELLHNV | 61 |
| FDASISQVNEKIN8SLAFIRXSDELLHNV | 62 |
| FDASISQVNEKINQ8LAFIRKXDELLHNV | 63 |
| FDASISQVNEKINQS8AFIRKSXELLHNV | 64 |
| FDASISQVNEKINQSL8FIRKSDXLLHNV | 65 |
| FDASISQVNEKINQSLA8IRKSDEXLHNV | 66 |
| FDASISQVNEKINQSLAF8RKSDELXHNV | 67 |
| FDASISQVNEKINQSLAFI8KSDELLXNV | 68 |
| FDASISQVNEKINQSLAFIRSSDELLHXV | 69 |
| FDASISQVNEKINQSLAFIRK8DELLHNX | 70 |

In Table 1, with respect to SEQ ID NOs: 27-48, “X₁″=α, α-disubstituted non-natural amino acid with an olefinic side chain (e.g., a non-natural amino acid substituted with an alpha-methyl group and an alpha-alkenyl group) cross-linked to X2; “X2″=α, α-disubstituted non-natural amino acid with olefinic side chain (e.g., alpha-methyl, alpha-alkenyl non-natural amino acid) cross-linked to X₁. In Table 1, with respect to SEQ ID NOs: 49-70, “8″=(R)-α-(7′-octenyl)alanine; “X”=(S)-α-(4′-pentenyl)alanine.

Note that the bolded residues in Table 1 identify the stapling amino acids. In some instances (e.g., a peptide described in Table 1), the structurally-stabilized peptide is single-stapled peptide.

The disclosure encompasses each and every peptide and structurally-stabilized peptide listed in Table 1 as well as variants thereof (e.g., having 1, 2, 3, 4, 5, 6, 7, 8, 9, 10, 11, 12, 13, 14, 15, 16) amino acid substitutions, insertions, and/or deletions, except at the positions of the staple (i.e., the bolded residues in Table 1). In some instances, the variant has 1 to 10 amino acid substitutions, insertions, and/or deletions, except at the positions of the staple (i.e., the bolded residues in Table 1). In some instances, the variant has 1 to 5 amino acid substitutions, insertions, and/or deletions, except at the positions of the staple, (i.e., the bolded residues in Table 1). In some instances, the variant has 1 to 3 amino acid substitutions, insertions, and/or deletions, except at the positions of the staple, (i.e., the bolded residues in Table 1). In some instances, the variant has 1 to 10, 10 to 5, 1 to 3, 2, or 1 amino acid substitutions, insertions, and/or deletions, except at the positions of the staple (i.e., the bolded residues in Table 1), relative to the amino acid sequence set forth in any one of SEQ ID NOs: 31-35 and 38. In some instances, the variant has 1 to 10, 10 to 5, 1 to 3, 2, or 1 amino acid substitutions, insertions, and/or deletions, except at the positions of the staple (i.e., the bolded residues in Table 1), relative to the amino acid sequence set forth in any one of SEQ ID NOs: 27, 29, 36, 43, and 46 or any one of SEQ ID NOs: 49, 51, 58, 65, and 68. In some instances, the structurally-stabilized peptide does not comprise the amino acid sequence NAGKST (SEQ ID NO:258). In some instances, the structurally-stabilized peptide does not comprise the amino acid sequence NAGKS (SEQ ID NO:259). In some instances, the structurally-stabilized peptide does not comprise the amino acid sequence NAGK (SEQ ID NO:260). In some instances, the structurally-stabilized peptide does not comprise the amino acid sequence NAG. In some instances, the structurally-stabilized peptide does not comprise the amino acid sequence NA. In some instances, the structurally-stabilized peptide does not comprise the amino acids corresponding to positions 517-522, 517-521, 517-520, 517-519, or 517-518 of SEQ ID NO:1 (or the corresponding amino acids of an RSV-F protein of another strain (see, e.g., FIG. 4)). In some instances, the structurally-stabilized peptide is 25 to 34 (e.g., 25, 26, 27, 28, 29, 30, 31, 32, 33, or 34) amino acids in length. In some instances, the structurally-stabilized is 30 amino acids in length. In some instances, the structurally-stabilized peptide has one or more (e.g., 1, 2, 3, 4, 5) of the following properties: (i) are alpha-helical; (ii) are protease resistant; (iii) bind to an RSV 5-helix bundle protein; (iv) inhibits infection of a cell by RSV; and/or (v) prevents infection of a cell by RSV. In some instances, the peptides inhibit infection of a cell by RSV in pseudovirus and/or live RSV virus assays and/or prevent infection of a cell by RSV in the pseudovirus and/or the live RSV virus assays.

In some instances, the structurally-stabilized peptide includes an amino acid sequence that has 2 to 10, 2 to 9, 2 to 8, 2 to 7, 2 to 6, 2 to 5, 2 to 4, 2 to 3, or 2 substitutions, insertions, and/or deletions relative to SEQ ID NO: 100. In some instances, a structurally-stabilized peptide having substitutions, insertions, and/or deletions relative to SEQ ID NO: 100 as described above (i) are alpha-helical; (ii) are protease resistant; (iii) bind to an RSV 5-helix bundle protein; (iv) inhibits infection of a cell by RSV; and/or (v) prevents infection of a cell by RSV. In some instances, the peptides inhibit infection of a cell by RSV in pseudovirus and/or live RSV virus assays and/or prevent infection of a cell by RSV in the pseudovirus and/or the live RSV virus assays.

In some instances, disclosed herein are peptides that comprise 0-10 (0, 1, 2, 3, 4, 5, 6, 7, 8, 9, 10) amino acid substitutions compared to one of the structurally-stabilized peptides in Table 1 (wherein the substitution(s) are not at the staple positions in Table 1). In some instances, disclosed herein are peptides that are at least at least 70%, at least 75%,at least 80%, at least 85%, at least 90%, or at lest 93% identical to one of the structurally-stabilized peptides in Table 1. In some instances, disclosed herein are peptides that are at least 70%, at least 75%,at least 80%, at least 85%, at least 90%, or at lest 93% identical to the amino acid sequence set forth in any one of SEQ ID NOs: 27, 29, 36, 43, 46 or any one of SEQ ID NOs:49, 51, 58, 65, and 68 (wherein the variation is not at the staple positions in SEQ ID NOs: 27, 29, 36, 43, and 46 or any one of SEQ ID NOs:49, 51, 58, 65, and 68, respectively). It is understood that the variation in a particular sequence is not at the staple position (i.e., the bolded residues in Table 1). In some instances, disclosed herein are peptides that are 100% identical to one of the structurally-stabilized peptides in Table 1. In some instances, disclosed herein are peptides that are 100% identical to the amino acid sequence set forth in any one of SEQ ID NOs: 27, 29, 36, 43, and 46 or any one of SEQ ID NOs:49, 51, 58, 65, and 68. In some instances, the structurally-stabilized peptide does not comprise the amino acid sequence NAGKST (SEQ ID NO:258). In some instances, the structurally-stabilized peptide does not comprise the amino acid sequence NAGKS (SEQ ID NO:259). In some instances, the structurally-stabilized peptide does not comprise the amino acid sequence NAGK (SEQ ID NO:260). In some instances, the structurally-stabilized peptide does not comprise the amino acid sequence NAG. In some instances, the structurally-stabilized peptide does not comprise the amino acid sequence NA. In some instances, the structurally-stabilized peptide does not comprise the amino acids corresponding to positions 517-522, 517-521, 517-520, 517-519, or 517-518 of SEQ ID NO:1 (or the corresponding amino acids of an RSV-F protein of another strain (see, e.g., FIG. 4)). In some instances, the structurally-stabilized peptide is 25 to 34 (e.g., 25, 26, 27, 28, 29, 30, 31, 32, 33, or 34) amino acids in length. In some instances, the structurally-stabilized is 30 amino acids in length. In some instances, the structurally-stabilized peptide has one or more (e.g., 1, 2, 3, 4, 5) of the following properties: (i) are alpha-helical; (ii) are protease resistant; (iii) bind to an RSV 5-helix bundle protein; (iv) inhibits infection of a cell by RSV; and/or (v) prevents infection of a cell by RSV. In some instances, the peptides inhibit infection of a cell by RSV in pseudovirus and/or live RSV virus assays and/or prevent infection of a cell by RSV in the pseudovirus and/or the live RSV virus assays.

In some instances, any substitution as described herein can be a conservative substitution. In some instances, any substitution as described herein is a non-conservative substitution.

In some instances, the structurally-stabilized peptide does not comprise the amino acid sequence NAGKST (SEQ ID NO:258). In some instances, the structurally-stabilized peptide does not comprise the amino acid sequence NAGKS (SEQ ID NO:259). In some instances, the structurally-stabilized peptide does not comprise the amino acid sequence NAGK (SEQ ID NO:260). In some instances, the structurally-stabilized peptide does not comprise the amino acid sequence NAG. In some instances, the structurally-stabilized peptide does not comprise the amino acid sequence NA. In some instances, the structurally-stabilized peptide does not comprise the amino acids corresponding to positions 517-522, 517-521, 517-520, 517-519, or 517-518 of SEQ ID NO:1 (or the corresponding amino acids of an RSV-F protein of another strain (see, e.g., FIG. 4)). In some instances, the structurally-stabilized peptide is 25 to 34 (e.g., 25, 26, 27, 28, 29, 30, 31, 32, 33, or 34) amino acids in length. In some instances, the structurally-stabilized is 30 amino acids in length.

In some instances, the non-natural amino acids that may be used as stapling amino acids are: (R)-2-(2′-propenyl)alanine; (R)-2-(4′-pentenyl)alanine; (R)-α-(7′-octenyl)alanine; (S)-α-(2′-propenyl)alanine; (S)-α-(4′-pentenyl)alanine; (S)-2-(7′-octenyl)alanine; α,α-Bis(4′-pentenyl)glycine; and α,α-Bis(7′-octeny)glycine.

In some instances, an internal staple replaces the side chains of 2 amino acids, i.e., each staple is between two amino acids separated by, for example, 6 amino acids. In some instances, the amino acids forming the staple are at each of positions i and i+7 of the staple. For example, where a peptide has the sequence . . . X1, X2, X3, X4, X5, X6, X7, X8, X9 . . . , cross-links between X1 and X8 (i and i+7) are useful hydrocarbon stapled forms of that peptide. The use of an i and i+4 staple, multiple cross-links (e.g., 2, 3, 4, or more), or a tandem stitch is also contemplated. Additional description regarding making and use of hydrocarbon-stapled peptides can be found, e.g., in U.S. Patent Publication Nos. 2012/0172285, 2010/0286057, and 2005/0250680, the contents of all of which are incorporated by reference herein in their entireties.

“Peptide stapling” is a term coined from a synthetic methodology wherein two olefin-containing side-chains (e.g., cross-linkable side chains) present in a peptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed. 37:3281, 1994). The structural-stabilization may be by, e.g., stapling the peptide (see, e.g., Walensky, J. Med. Chem., 57:6275-6288 (2014), the contents of which are incorporated by reference herein in its entirety). In some cases, the staple is a hydrocarbon staple.

In some instances, a staple used herein is an all hydrocarbon staple.

In some instances, a staple used herein is a lactam staple; a UV-cycloaddition staple; an oxime staple; a thioether staple; a double-click staple; a bis-lactam staple; a bis-arylation staple; or a combination of any two or more thereof. Stabilized peptides as described herein include stapled peptides as well as peptides containing multiple staples or any other chemical strategies for structural reinforcement (see. e.g., Balaram P. Cur. Opin. Struct. Biol. 1992; 2:845; Kemp D S, et al., J. Am. Chem. Soc. 1996; 118:4240; Orner B P, et al., J. Am. Chem. Soc. 2001; 123:5382; Chin J W, et al., Int. Ed. 2001; 40:3806; Chapman R N, et al., J. Am. Chem. Soc. 2004; 126:12252; Horne W S, et al., Chem., Int. Ed. 2008; 47:2853; Madden et al., Chem Commun (Camb). 2009 Oct. 7; (37): 5588-5590; Lau et al., Chem. Soc. Rev., 2015,44:91-102; and Gunnoo et al., Org. Biomol. Chem., 2016,14:8002-8013; each of which is incorporated by reference herein in its entirety).

A peptide is “structurally-stabilized” in that it maintains its native secondary structure. For example, stapling allows a peptide, predisposed to have an a-helical secondary structure, to maintain its native a-helical conformation. This secondary structure increases resistance of the peptide to proteolytic cleavage and heat, and may increase target binding affinity, hydrophobicity, plasma membrane binding, and/or cell permeability. Accordingly, the stapled (cross-linked) peptides described herein have improved biological activity and pharmacology relative to a corresponding non-stapled (un-cross-linked) peptide.

In some instances, a structurally-stabilized peptide described herein comprises an amino acid sequence comprising 25 to 29 contiguous amino acids of the sequence of SEQ ID NO:100, except for two to six amino acid substitutions relative to the sequence of SEQ ID NO:100; wherein the structurally-stabilized peptide comprises an internally cross-linked amino acid sequence having the formula:

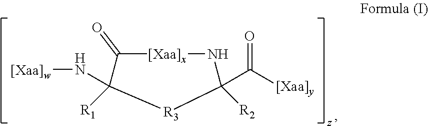

or a pharmaceutically acceptable salt thereof;
wherein each R1 and R2 is H or a C1 to C10 alkyl, alkenyl, alkynyl, arylalkyl, cycloalkylalkyl, heteroarylalkyl, or heterocyclylalkyl, any of which is substituted or unsubstituted;
wherein x is 3 or 6;
wherein each R₃ is independently alkylene, alkenylene, or alkynylene, any of which is substituted or unsubstituted;
wherein z is 1, 2, 3, 4, 5, 6, 7, 8, 9, or 10;
wherein the structurally-stabilized peptide binds to an RSV 5-helix bundle protein and/or
wherein the structurally-stabilized peptide inhibits infection of a cell by RSV and/or prevents infection of a cell by RSV; and
wherein the structurally-stabilized peptide is 25 to 30 amino acids in length, optionally
wherein the structurally-stabilized peptide is 29 amino acids in length. In some instances, each [Xaa]ₓ is an [Xaa]ₓ identified in Table 2, or a variant thereof having one amino acid substitution. In some instances, the amino acid sequence comprises 25 to 30 (e.g., 25, 26, 27, 28, 29, 30) contiguous amino acids of the sequence set forth in SEQ ID NO:100 with 2 to 5 (e.g., 2, 3, 4, 5) amino acid substitutions relative to the sequence set forth in SEQ ID NO:100. In some instances, the amino acid sequence comprises 25 to 30 (e.g., 25, 26, 27, 28, 29, 30) contiguous amino acids of the sequence set forth in SEQ ID NO:100 with 2 to 4 (e.g., 2, 3, 4) amino acid substitutions relative to the sequence set forth in SEQ ID NO:100. In some instances, the amino acid sequence comprises 25 to 30 (e.g., 25, 26, 27, 28, 29, 30) contiguous amino acids of the sequence set forth in SEQ ID NO:100 with 2 or 3 amino acid substitutions relative to the sequence set forth in SEQ ID NO: 100. In some instances, the amino acid sequence comprises 25 to 30 (e.g., 25, 26, 27, 28, 29, 30) contiguous amino acids of the sequence set forth in SEQ ID NO:100 with 2 amino acid substitutions relative to the sequence set forth in SEQ ID NO:100. In some instances, the structurally-stabilized peptide does not comprise the amino acid sequence NAGKST (SEQ ID NO:258). In some instances, the structurally-stabilized peptide does not comprise the amino acid sequence NAGKS (SEQ ID NO:259). In some instances, the structurally-stabilized peptide does not comprise the amino acid sequence NAGK (SEQ ID NO:260). In some instances, the structurally-stabilized peptide does not comprise the amino acid sequence NAG. In some instances, the structurally-stabilized peptide does not comprise the amino acid sequence NA. In some instances, the structurally-stabilized peptide does not comprise the amino acids corresponding to positions 517-522, 517-521, 517-520, 517-519, or 517-518 of SEQ ID NO:1 (or the corresponding amino acids of an RSV-F protein of another strain (see, e.g., FIG. 4)). In some instances, the structurally-stabilized peptide described above has one or more (e.g., 1, 2, 3, 4, 5) of the following properties: (i) are alpha-helical; (ii) are protease resistant; (iii) bind to an RSV 5-helix bundle protein; (iv) inhibits infection of a cell by RSV; and/or (v) prevents infection of a cell by RSV. In some instances, the peptides inhibit infection of a cell by RSV in pseudovirus and/or live RSV virus assays and/or prevent infection of a cell by RSV in the pseudovirus and/or the live RSV virus assays.

In certain instances, substitutions to the contiguous amino acid sequence of SEQ ID NO:100 in Formula I (other than the substitutions to introduce the linking group R₃) are conservative. In certain instances, substitutions to the contiguous amino acid sequence of SEQ ID NO:100 in Formula I (other than the substitutions to introduce the linking group R₃) are non-conservative. Methods for determining the type of substitution are described herein, see, e.g., the “RSV Peptides” section above. In some instances, the structurally-stabilized peptide is 25 to 45 (e.g., 25, 26, 27, 28, 29, 30, 31, 32, 33, 34, 35, 36, 37, 38, 39, 40, 41, 42, 43, 44, 45) amino acids in length. In some instances, the structurally-stabilized peptide is 25 to 40 (e.g., 25, 26, 27, 28, 29, 30, 31, 32, 33, 34, 35, 36, 37, 38, 39, 40) amino acids in length. In some instances, the structurally-stabilized peptide is 25 to 35 (e.g., 25, 26, 27, 28, 29, 30, 31, 32, 33, 34, 35) amino acids in length. In some instances, the structurally-stabilized peptide is 25 to 30 (e.g., 25, 26, 27, 28, 29, 30) amino acids in length. In some instances, the structurally-stabilized peptide is 30 amino acids in length. In some instances, the structurally-stabilized peptide described above has one or more (e.g., 1, 2, 3, 4, 5) of the following properties: (i) are alpha-helical; (ii) are protease resistant; (iii) bind to an RSV 5-helix bundle protein; (iv) inhibits infection of a cell by RSV; and/or (v) prevents infection of a cell by RSV. In some instances, the peptides inhibit infection of a cell by RSV in pseudovirus and/or live RSV virus assays and/or prevent infection of a cell by RSV in the pseudovirus and/or the live RSV virus assays.

In some instances of Formula (I), each R₁ and R₂ are independently H or a C₁ to C₁₀ alkyl, alkenyl, alkynyl, arylalkyl, cycloalkylalkyl, heteroarylalkyl, or heterocyclylalkyl;
R₃ is alkyl, alkenyl, alkynyl; [R₄-K-R₄]ₙ; each of which is substituted with 0-6 R₅;
R₄ is alkyl, alkenyl, or alkynyl;
R₅ is halo, alkyl, OR₆, N(R₆)₂, SR₆, SOR₆, SO₂R₆, CO₂R₆, R₆, a fluorescent moiety, or a radioisotope;
K is O, S, SO, SO₂, CO, CO₂, CONR₆, or

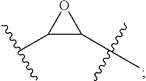 [SMILES: *C1OC1*]

R₆ is H, alkyl, or a therapeutic agent;
n is an integer from 1-4;
x is 3 or 6;
each y is independently an integer from 0-100;
z is an integer from 1-10 (e.g., 1, 2, 3, 4, 5, 6, 7, 8, 9, 10);
and each Xaa is independently an amino acid.

In some instances, each of the [Xaa]w of Formula (I), the [Xaa]ₓ of Formula (I), and the [Xaa]_{y} of Formula (I) is as described for any one of constructs 1-22 of Table 2. For example, for a structurally-stabilized peptide comprising the [Xaa]w, the [Xaa]ₓ, and the [Xaa]_{y} of construct 3 of Table 2, the [Xaa]_{w}, the [Xaa]ₓ, and the [Xaa]_{y} are: FD, SISQVN (SEQ ID NO:220), and KINQSLAFIRKSDELLHNV (SEQ ID NO:242), respectively.

TABLE 2
[Xaa]w, [Xaa]x, and [Xaa]y sequences for Formula (I) constructs 1-22.

| Construct | [Xaa]w | [Xaa]x | [Xaa]y |
|---|---|---|---|
| 1 | Absent | DASISQ (SEQ ID | NEKINQSLAFIRKSDE |
| | | NO: 218) | LLHNV (SEQ ID |
| | | | NO: 240) |
| 2 | F | ASISQV (SEQ ID | EKINQSLAFIRKSDELL |
| | | NO: 219) | HNV (SEQ ID NO: 241) |
| 3 | FD | SISQVN (SEQ ID | KINQSLAFIRKSDELLH |
| | | NO: 220) | NV (SEQ ID NO: 242) |
| 4 | FDA | ISQVNE (SEQ ID | INQSLAFIRKSDELLHN |
| | | NO: 221) | V (SEQ ID NO: 243) |
| 5 | FDAS | SQVNEK (SEQ ID | NQSLAFIRKSDELLHN |
| | (SEQ ID NO: 200) | NO: 222) | V (SEQ ID NO: 244) |
| 6 | FDASI (SEQ ID | QVNEKI (SEQ ID | QSLAFIRKSDELLHNV |
| | NO: 201) | NO: 223) | (SEQ ID NO: 245) |
| 7 | FDASIS (SEQ ID | VNEKIN (SEQ ID | SLAFIRKSDELLHNV |
| | NO: 202) | NO: 224) | (SEQ ID NO: 246) |
| 8 | FDASISQ (SEQ ID | NEKINQ (SEQ ID | LAFIRKSDELLHNV |
| | NO: 203) | NO: 225) | (SEQ ID NO: 247) |
| 9 | FDASISQV (SEQ ID | EKINQS (SEQ ID | AFIRKSDELLHNV |
| | NO: 204) | NO: 226) | (SEQ ID NO: 248) |
| 10 | FDASISQVN (SEQ ID | KINQSL (SEQ ID | FIRKSDELLHNV (SEQ |
| | NO: 205) | NO: 227) | ID NO: 249) |
| 11 | FDASISQVNE (SEQ ID | INQSLA (SEQ ID | IRKSDELLHNV (SEQ |
| | NO: 206) | NO: 228) | ID NO: 250) |
| 12 | FDASISQVNEK (SEQ | NQSLAF (SEQ ID | RKSDELLHNV (SEQ ID |
| | ID NO: 207) | NO: 229) | NO: 251) |
| 13 | FDASISQVNEKI (SEQ | QSLAFI (SEQ ID | KSDELLHNV (SEQ ID |
| | ID NO: 208) | NO: 230) | NO: 252) |
| 14 | FDASISQVNEKIN | SLAFIR(SEQ ID | SDELLHNV (SEQ ID |
| | (SEQ ID NO: 209) | NO: 231) | NO: 253) |
| 15 | FDASISQVNEKINQ | LAFIRK (SEQ ID | DELLHNV (SEQ ID |
| | (SEQ ID NO: 210) | NO: 232) | NO: 254) |
| 16 | FDASISQVNEKINQS | AFIRKS (SEQ ID | ELLHNV (SEQ ID |
| | (SEQ ID NO: 211) | NO: 233) | NO: 255) |
| 17 | FDASISQVNEKINQSL | FIRKSD (SEQ ID | LLHNV (SEQ ID |
| | (SEQ ID NO: 212) | NO: 234) | NO: 256) |
| 18 | FDASISQVNEKINQSL | IRKSDE (SEQ ID | LHNV (SEQ ID |
| | A (SEQ ID NO: 213) | NO: 235) | NO: 257) |
| 19 | FDASISQVNEKINQSL | RKSDEL (SEQ ID | HNV |
| | AF (SEQ ID NO: 214) | NO: 236) | |
| 20 | FDASISQVNEKINQSL | KSDELL (SEQ ID | NV |
| | AFI (SEQ ID NO: 215) | NO: 237) | |
| 21 | FDASISQVNEKINQSL | SDELLH (SEQ ID | V |
| | AFIR (SEQ ID NO: 216) | NO: 238) | |
| 22 | FDASISQVNEKINQSL | DELLHN (SEQ ID | Absent |
| | AFIRK (SEQ ID | NO: 239) | |
| | NO: 217) | | |

In some instances, the structurally-stabilized peptide comprises or consists of construct 3 of Table 2. In some instances, the structurally-stabilized peptide comprises or consists of construct 17 of Table 2. In some instances, the structurally-stabilized peptide comprises or consists of any one of constructs 1-22 of Table 2 except for at least one (e.g., 1, 2, 3, 4, 5, or 6) amino acid substitution or deletion (e.g., up to a total of 2 to 10 (e.g., 2, 3, 4, 5, 6, 7, 8, 9, 10) substitutions or deletions relative to the sequence of any one of Constructs 1-22, respectively).

In certain instances, the sequences set forth above in Table 2 can have at least one (e.g., 1, 2, 3, 4, 5, or 6) amino acid substitution or deletion (e.g., up to a total of 2 to 10 (e.g., 2, 3, 4, 5, 6, 7, 8, 9, 10) substitutions or deletions relative to the sequence of SEQ ID NO: 100). The RSV-F HR2 peptides can include any amino acid sequence described herein.

In some instances, the structurally-stabilized peptide of Formula (I) comprises the sequences of a construct set forth above in Table 2 and has one or more (e.g., 1, 2, 3, 4, 5) of the following properties: (i) are alpha-helical; (ii) are protease resistant; (iii) bind to an RSV 5-helix bundle protein; (iv) inhibits infection of a cell by RSV; and/or (v) prevents infection of a cell by RSV. In some instances, the structurally-stabilized peptide inhibit infection of a cell by RSV in pseudovirus and/or live RSV virus assays and/or prevent infection of a cell by RSV in the pseudovirus and/or the live RSV virus assays.

The tether of Formula (I) can include an alkyl, alkenyl, or alkynyl moiety (e.g., C₅, C₈, C₁₁, or C₁₂ alkyl, a C₅, C₈, or C₁₁ alkenyl, or C₅, C₈, C₁₁, or C₁₂ alkynyl). The tethered amino acid can be alpha disubstituted (e.g., C₁-C₃ or methyl).

In some instances of Formula (I), each y is independently an integer between 0 and 15, or 3 and 15. In some instances of Formula (I), R₁ and R₂ are each independently H or C₁-C₆ alkyl. In some instances of Formula (I), R₁ and R₂ are each independently C₁-C₃ alkyl. In some instances or Formula (I), at least one of R₁ and R₂ are methyl. For example, R₁ and R₂ can both be methyl. In some instances of Formula (I), R₃ is C₁₁ alkyl and x is 6. In some instances of Formula (I), x is 6 and R₃ is C₁₁ alkenyl. In some instances, R₃ is a straight chain alkyl, alkenyl, or alkynyl. In some instances, R₃ is —CH₂—CH₂—CH₂—CH═CH—CH₂—CH₂—CH₂.

In another aspect of Formula (I), the two alpha, alpha disubstituted stereocenters are both in the R configuration or S configuration (e.g., i, i+4 cross-link), or one stereocenter is R and the other is S (e.g., i, i i 7 cross-link). Thus, where Formula (I) is depicted as:

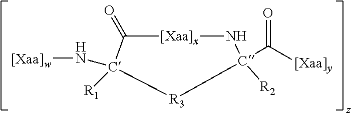

The C′ and C″ disubstituted stereocenters can both be in the R configuration or they can both be in the S configuration. When x is 6 in Formula (I), the C′ disubstituted stereocenter is in the R configuration and the C″ disubstituted stereocenter is in the S configuration. The R₃ double bond of Formula (I) can be in the E or Z stereochemical configuration.

In some instances of Formula (I), R₃ is [R₄ K R₄]ₙ; and R₄ is a straight chain alkyl, alkenyl, or alkynyl.

As used herein, the term “alkyl,” employed alone or in combination with other terms, refers to a saturated hydrocarbon group that may be straight-chain or branched. In some instances, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some instances, the alkyl group is methyl, ethyl, or propyl. The term “alkylene” refers to a linking alkyl group.

As used herein, “alkenyl,” employed alone or in combination with other terms, refers to an alkyl group having one or more carbon-carbon double bonds. In some instances, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some instances, the alkynyl moiety contains 2 to 6 or 2 to 4 carbon atoms.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some instances, the alkynyl moiety contains 2 to 6 or 2 to 4 carbon atoms.

As used herein, the term “cycloalkylalkyl,” employed alone or in combination with other terms, refers to a group of formula cycloalkyl-alkyl-. In some instances, the alkyl portion has 1 to 4, 1 to 3, 1 to 2, or 1 carbon atom(s). In some instances, the alkyl portion is methylene. In some instances, the cycloalkyl portion has 3 to 10 ring members or 3 to 7 ring members. In some instances, the cycloalkyl group is monocyclic or bicyclic. In some instances, the cycloalkyl portion is monocyclic. In some instances, the cycloalkyl portion is a C₃₋₇ monocyclic cycloalkyl group.

As used herein, the term “heteroarylalkyl,” employed alone or in combination with other terms, refers to a group of formula heteroaryl-alkyl-. In some instances, the alkyl portion has 1 to 4, 1 to 3, 1 to 2, or 1 carbon atom(s). In some instances, the alkyl portion is methylene. In some instances, the heteroaryl portion is a monocyclic or bicyclic group having 1, 2, 3, or 4 heteroatoms independently selected from nitrogen, sulfur and oxygen. In some instances, the heteroaryl portion has 5 to 10 carbon atoms.

As used herein, the term “substituted” means that a hydrogen atom is replaced by a non-hydrogen group. It is to be understood that substitution at a given atom is limited by valency.

As used herein, “halo” or “halogen”, employed alone or in combination with other terms, includes fluoro, chloro, bromo, and iodo. In some instances, halo is F or Cl.

While hydrocarbon tethers are provided herein, other tethers can also be employed in the structurally-stabilized RSV-F HR2 peptides described herein. For example, the tether can include one or more of an ether, thioether, ester, amine, or amide, or triazole moiety. In some cases, a naturally occurring amino acid side chain can be incorporated into the tether. For example, a tether can be coupled with a functional group such as the hydroxyl in serine, the thiol in cysteine, the primary amine in lysine, the acid in aspartate or glutamate, or the amide in asparagine or glutamine. Accordingly, it is possible to create a tether using naturally occurring amino acids rather than using a tether that is made by coupling two non-naturally occurring amino acids. It is also possible to use a single non-naturally occurring amino acid together with a naturally occurring amino acid. Triazole-containing (e.g., 1, 4 triazole or 1, 5 triazole) crosslinks can be used (see, e.g., Kawamoto et al. 2012 Journal of Medicinal Chemistry 55:1137; WO 2010/060112). In addition, other methods of performing different types of stapling are well known in the art and can be employed with the RSV-F HR2 peptides described herein (see, e.g., Lactam stapling: Shepherd et al., J. Am. Chem. Soc., 127:2974-2983 (2005); UV-cycloaddition stapling: Madden et al., Bioorg. Med. Chem. Lett., 21:1472-1475 (2011); Disulfide stapling: Jackson et al., Am. Chem. Soc.,113:9391-9392 (1991); Oxime stapling: Haney et al., Chem. Commun., 47:10915-10917 (2011); Thioether stapling: Brunel and Dawson, Chem. Commun., 552-2554 (2005); Photoswitchable stapling: J. R. Kumita et al., Proc. Natl. Acad. Sci. U.S.A, 97:3803-3808 (2000); Double-click stapling: Lau et a/., Chem. Sci., 5:1804-1809 (2014); Bis-lactam stapling: J. C. Phelan et al., J. Am. Chem. Soc., 119:455-460 (1997); and Bis-arylation stapling: A. M. Spokoyny et al., J. Am. Chem. Soc., 135:5946-5949 (2013)).

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

Additionally, while tethers spanning from amino acids i to i+7 are provided herein in order to provide a tether that is primarily on a single face of the alpha helix, the tethers can be synthesized to span any combinations of numbers of amino acids and also used in combination to install multiple tethers.

In some instances, the hydrocarbon tethers (i.e., cross links) described herein can be further manipulated. In one instance, a double bond of a hydrocarbon alkenyl tether, (e.g., as synthesized using a ruthenium-catalyzed ring closing metathesis (RCM)) can be oxidized (e.g., via epoxidation, aminohydroxylation or dihydroxylation) to provide one of compounds below.

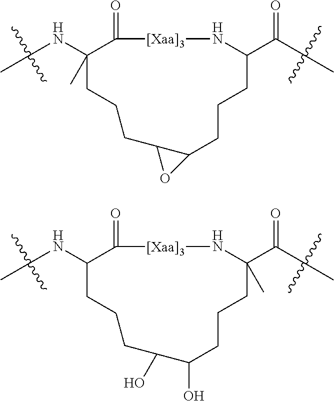

Either the epoxide moiety or one of the free hydroxyl moieties can be further functionalized. For example, the epoxide can be treated with a nucleophile, which provides additional functionality that can be used, for example, to attach a therapeutic agent. Such derivatization can alternatively be achieved by synthetic manipulation of the amino or carboxy-terminus of the peptide or via the amino acid side chain. Other agents can be attached to the functionalized tether, e.g., an agent that facilitates entry of the peptide into cells.

In some instances, alpha disubstituted amino acids are used in the peptide to improve the stability of the alpha helical secondary structure. However, alpha disubstituted amino acids are not required, and instances using mono-alpha substituents (e.g., in the tethered amino acids) are also envisioned.

The structurally-stabilized (e.g., stapled) peptides can include a drug, a toxin, a derivative of polyethylene glycol; a second peptide; a carbohydrate, etc. Where a polymer or other agent is linked to the structurally-stabilized (e.g., stapled) peptide, it can be desirable for the composition to be substantially homogeneous.

The structurally-stabilized (e.g., stapled) peptides can also be modified, e.g., to further facilitate mucoadhesion, membrane binding, or increase in vivo stability, in some instances. For example, acylating or PEGylating a structurally-stabilized peptide increases bioavailability, increases blood circulation, alters pharmacokinetics, alters immunogenicity and/or decreases the needed frequency of administration.

In some instances, the structurally-stabilized (e.g., stapled) peptides disclosed herein have an enhanced ability to bind to or penetrate cell membranes (e.g., relative to non-stabilized peptides). See, e.g., International Publication No. WO 2017/147283, which is incorporated by reference herein in its entirety.

In some instances, the structurally-stabilized peptide is a peptide described in the figures or in the working examples.

In some instances, a structurally-stabilized peptide described herein (e.g., in Table 1) forms part of a structurally-stabilized peptide conjugate described in the “Structurally-Stabilized Peptide Conjugates” section below.

### Structurally-Stabilized Peptide Conjugates

Also provided herein are conjugates comprising a structurally-stabilized peptide described herein (see “Structurally-Stabilized Peptides” section above, e.g., a peptide of Table 1 or a construct of Table 2, or a variant thereof) and cholesterol (or a cholesterol variant, e.g., thiocholesterol), wherein the cholesterol (or a cholesterol variant, e.g., thiocholesterol) is linked, directly or via a linker (e.g., PEG(n), wherein n 4, 5, 6, 7, 8,9, 10, 11, 12, 13, 14, 15, 16, 17, 18, 19, 20), to the C-terminal amino acid of the structurally-stabilized peptide. “Conjugates” and “structurally-stabilized peptide conjugates” are referred to interchangeably herein. In some instances, the conjugate comprises the structurally-stabilized peptide and the cholesterol, wherein the cholesterol is linked to, directly or via a linker (e.g., PEG(n), wherein n 4, 5, 6, 7, 8,9, 10, 11, 12, 13, 14, 15, 16, 17, 18, 19, 20). In some instances, the conjugate comprises the structurally-stabilized peptide and the thiocholesterol, wherein the thiocholesterol is linked to, directly or via a linker (e.g., PEG(n), wherein n 4, 5, 6, 7, 8,9, 10, 11, 12, 13, 14, 15, 16, 17, 18, 19, 20). The conjugates have one or more (e.g., 1, 2, 3, 4, 5) of the properties listed below: (i) are alpha-helical; (ii) are protease resistant; (iii) bind to an RSV 5-helix bundle protein; (iv) inhibits infection of a cell by RSV; and/or (v) prevents infection of a cell by RSV. In some instances of the conjugates, the structurally-stabilized peptide is conjugated to thiocholesterol. In some instances of the conjugates, the structurally-stabilized peptide is conjugated to thiocholesterol via a linker comprising PEG (e.g., PEG(n), wherein n 4, 5, 6, 7, 8,9, 10, 11, 12, 13, 14, 15, 16, 17, 18, 19, 20). In some instances of the conjugates, the structurally-stabilized peptide is conjugated to cholesterol. In some instances of the conjugates, the structurally-stabilized peptide is conjugated to cholesterol via a linker comprising PEG (e.g., PEG(n), wherein n 4, 5, 6, 7, 8,9, 10, 11, 12, 13, 14, 15, 16, 17, 18, 19, 20). In some instances, the structurally-stabilized peptide of the conjugate is a structurally-stabilized version of a peptide described in the “RSV Peptides” section above. In some instances, the structurally-stabilized peptide of the conjugate is a structurally-stabilized peptide described in the “Structurally-Stabilized Peptides” section above. In some instances, the structurally-stabilized peptide of the conjugate is a structurally-stabilized peptide described in the working examples or figures herein.

In some instances, the structurally-stabilized peptide of the conjugate comprises an internally cross-linked amino acid sequence comprising 25 to 29 contiguous amino acids of the sequence FDASISQVNEKINQSLAFIRKSDELLHNV (SEQ ID NO:100), except for two to six amino acid substitutions relative to the sequence of SEQ ID NO:100 and wherein the internally cross-linked amino acid sequence does not include the sequence NAGKST (SEQ ID NO:258); wherein two of the two to six amino acid substitutions are with α, α-disubstituted non-natural amino acids with olefinic side chains cross-linked to each other, wherein the α, α-disubstituted non-natural amino acids with olefinic side chains cross-linked to each other are separated by three or six amino acids; wherein the conjugate binds to an RSV 5-helix bundle protein and/or wherein the conjugate RSV 5-helix bundle protein and/or prevents infection of a cell by RSV; and wherein the conjugate is 25 to 45 amino acids (e.g., 25, 26, 27, 28, 29, 30, 31, 32, 33, 34, 35, 36, 37, 38, 39, 40, 41, 42, 43, 44, 45) in length.

In some instances, the structurally-stabilized peptide of the conjugate comprises or consists of an amino acid sequence in Table 1 (e.g., any one SEQ ID NOs:27-70). In some instances, the structurally-stabilized peptide of the conjugate comprises or consists of the amino acid sequence of any one of SEQ ID NOs:27, 29, 36, 43, and 46 or any one of SEQ ID NOs:49, 51, 58, 65, and 68. In some instances, the structurally-stabilized peptide of the conjugate comprises or consists of the amino acid sequence of SEQ ID NO:29. In some instances, the structurally-stabilized peptide of the conjugate comprises or consists of the amino acid sequence of SEQ ID NO:51. In some instances, the structurally-stabilized peptide of the conjugate comprises or consists of the amino acid sequence of SEQ ID NO:43. In some instances, the structurally-stabilized peptide of the conjugate comprises or consists of the amino acid sequence of SEQ ID NO:65. In some instances, the structurally-stabilized peptide of the conjugate comprises or consists of any one of Constructs of Table 2. In some instances, the structurally-stabilized peptide of the conjugate comprises or consists of any one of constructs 1, 3, 10, 17, or 20 of Table 2. In some instances, the structurally-stabilized peptide of the conjugate comprises or consists of construct 3 of Table 2. In some instances, the structurally-stabilized peptide of the conjugate comprises or consists of construct 17 of Table 2. In some instances, the structurally-stabilized peptide of the conjugate comprises or consists of the amino acid sequence of any one of the peptides of Table 1 except for 1 to 10, 1 to 5, 1 to 3, 2, or 1 amino acid substitutions, insertions, and/or deletions (except at the positions of the staple). In some instances, the structurally-stabilized peptide of the conjugate comprises or consists of any one of the constructs of Table 2 except for 1 to 10, 1 to 5, 1 to 3, 2, or 1 amino acid substitutions, insertions, and/or deletions (except at the position of the staple).

The addition of PEG molecules can improve the pharmacokinetic and pharmacodynamic properties of the structurally-stabilized peptide. For example, PEGylation can reduce renal clearance and can result in a more stable plasma concentration. PEG is a water soluble polymer and can be represented as linked to the peptide as formula:

XO—(CH₂CH₂O)ₙ—CH₂CH₂—Y

where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the structurally-stabilized peptide. Y may also be a maleimide linkage to a thiol group (including but not limited to, the thiol group of cysteine). Other methods for linking PEG to a peptide, directly or indirectly, are known to those of ordinary skill in the art. The PEG can be linear or branched. Various forms of PEG including various functionalized derivatives are commercially available.

PEG as used herein in some instances functions as a linker or spacer between one of the peptides (e.g., structurally-stabilized peptides of Table 1 or constructs of Table 2) and a cholesterol or thiocholesterol moiety.

In some instances, the PEG molecule includes a cholesterol moiety. In some instances, the cholesterol moiety is thiocholesterol. In some instances, the sulfur of the thioether moiety in thiocholesterol is replaced by an oxygen atom to produce an ether moiety in the cholesterol derivatization.

In some instances, the PEG molecule comprises the following formula (Formula II), wherein n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36):

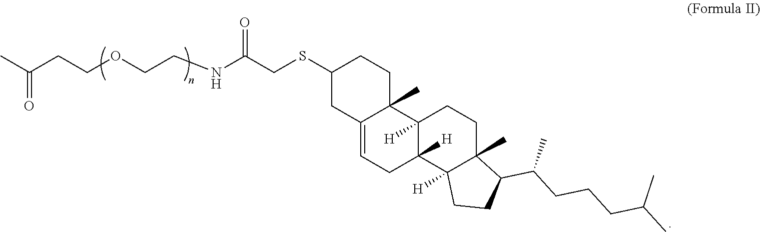

In some instances, the PEG molecule comprises the following formula (Formula III), wherein n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36):

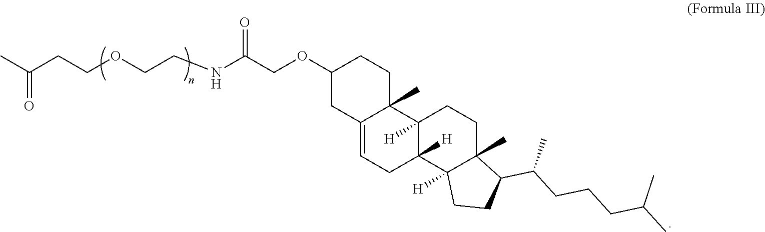

In some instances for each of Formula II and III, n=16. In some instances for each of Formula II and III, n=17. In some instances for each of Formula II and III, n =18. In some instances for each of Formula II and III, n=19. In some instances for each of Formula II and III, n=20.

PEG having degradable linkages in the backbone can be used. For example, PEG can be prepared with ester linkages that are subject to hydrolysis. Degradable PEG linkages are described in WO 99/34833; WO 99/14259, and U.S. Pat. No. 6,348,558, each of which is incorporated by reference herein in its entirety.

In certain instances, macromolecular polymer (e.g., PEG) is attached to a structurally-stabilized (e.g., stapled) peptide described herein through an intermediate linker. In certain instances, the linker is made up of from I to 20 amino acids linked by peptide bonds, wherein the amino acids are selected from the 20 naturally occurring amino acids. Some of these amino acids may be glycosylated, as is well understood by those in the art. In other instances, the 1 to 20 amino acids are selected from glycine, alanine, proline, asparagine, glutamine, and lysine. In other instances, a linker is made up of a majority of amino acids that are sterically unhindered, such as glycine and alanine. Non-peptide linkers are also possible. For example, alkyl linkers such as —NH(CH₂)ₙC(O)—, wherein n=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. U.S. Pat. No. 5,446,090 describes a bifunctional PEG linker and its use in forming conjugates having a peptide at each of the PEG linker termini.

Exemplary structurally-stabilized peptide conjugates are provided in Table 3, below. It is noted that the disclosure also encompasses structurally stabilized peptide conjugates of Tables 3further comprising three N-terminal amino acids X1X2X3, wherein X1 is any amino acid, optionally S; X2 and X3 are any negatively charged amino acid immediately upstream of the first amino acid of the sequences listed in Table 3. In some cases XIX2X3=SDE. In some instances, the structurally-stabilized peptide conjugate comprises or consists of the amino acid sequence set forth in any one of SEQ ID NOs:71-92. In some instances, the structurally-stabilized peptide conjugate comprises or consists of the amino acid sequence set forth in any one of SEQ ID NOs:6-26. In some instances, the structurally-stabilized peptide conjugate comprises or consists of the sequence set forth in SEQ ID NO: 5 or SEQ ID NO:73. In some instances, the structurally-stabilized peptide conjugate comprises or consists of the sequence set forth in SEQ ID NO: 21 or SEQ ID NO:87. In some instances, the structurally-stabilized peptide conjugate comprises or consists of the amino acid sequence set forth in any one of SEQ ID NOs:71-92 except for 1 to 10, 1 to 5, 1 to 3, 2, or 1 amino acid substitutions, insertions, and/or deletions (except at the position of the staple and at the position of the conjugation). In some instances, the structurally-stabilized peptide conjugate comprises or consists of the amino acid sequence set forth in any one of SEQ ID NOs:6-26 except for 1 to 10, 1 to 5, 1 to 3, 2, or 1 amino acid substitutions, insertions, and/or deletions (except at the position of the staple and at the position of the conjugation). In some instances, the structurally-stabilized peptide conjugate has one or more (e.g., 1, 2, 3, 4, 5) of the properties listed below: (i) are alpha-helical; (ii) are protease resistant; (iii) bind to an RSV 5-helix bundle protein; (iv) inhibits infection of a cell by RSV; and/or (v) prevents infection of a cell by RSV.

TABLE 3
Structurally-Stabilized RSV-F HR2 Peptide Conjugates

| | SEQ ID | | SEQ ID |
|---|---|---|---|
| Sequence | NO | Sequence | NO |
| X₁DASISQX₂NEKINQSLAFIRKSDELLHNV* | 71 | 8DASISQXNEKINQSLAFIRKSDELLHNV* | 6 |
| FX₁ASISQVX₂EKINQSLAFIRKSDELLHNV* | 72 | F8ASISQVXEKINQSLAFIRKSDELLHNV* | 7 |
| FDX₁SISQVNX₂KINQSLAFIRKSDELLHNV* | 73 | FD8SISQVNXKINQSLAFIRKSDELLHNV* | 5 |
| FDAX₁ISQVNEX₂INQSLAFIRKSDELLHNV* | 74 | FDA8ISQVNEXINQSLAFIRKSDELLHNV* | 8 |
| FDASX₁SQVNEKX₂NQSLAFIRKSDELLHNV* | 75 | FDAS8SQVNEKXNQSLAFIRKSDELLHNV* | 9 |
| FDASIX₁QVNEKIX₂QSLAFIRKSDELLHNV* | 76 | FDASI8QVNEKIXQSLAFIRKSDELLHNV* | 10 |
| FDASISX₁VNEKINX₂SLAFIRKSDELLHNV* | 77 | FDASIS8VNEKINXSLAFIRKSDELLHNV* | 11 |
| FDASISQX₁NEKINQX₂LAFIRKSDELLHNV* | 78 | FDASISQ8NEKINQXLAFIRKSDELLHNV* | 12 |
| FDASISQVX₁EKINQSX₂AFIRKSDELLHNV* | 79 | FDASISQV8EKINQSXAFIRKSDELLHNV* | 13 |
| FDASISQVNX₁KINQSLX₂FIRKSDELLHNV* | 80 | FDASISQVN8KINQSLXFIRKSDELLHNV* | 14 |
| FDASISQVNEX₁INQSLAX₂IRKSDELLHNV* | 81 | FDASISQVNE8INQSLAXIRKSDELLHNV* | 15 |
| FDASISQVNEKX₁NQSLAFX₂RKSDELLHNV* | 82 | FDASISQVNEK8NQSLAFXRKSDELLHNV* | 16 |
| FDASISQVNEKIX₁QSLAFIX₂KSDELLHNV* | 83 | FDASISQVNEKI8QSLAFIXKSDELLHNV* | 17 |
| FDASISQVNEKINX₁SLAFIRX₂SDELLHNV* | 84 | FDASISQVNEKIN8SLAFIRXSDELLHNV* | 18 |
| FDASISQVNEKINQX₁LAFIRKX₂DELLHNV* | 85 | FDASISQVNEKINQ8LAFIRKXDELLHNV* | 19 |
| FDASISQVNEKINQSX₁AFIRKSX₂ELLHNV* | 86 | FDASISQVNEKINQS8AFIRKSXELLHNV* | 20 |
| FDASISQVNEKINQSLX₁FIRKSDX₂LLHNV* | 87 | FDASISQVNEKINQSL8FIRKSDXLLHNV* | 21 |
| FDASISQVNEKINQSLAX₁IRKSDEX₂LHNV* | 88 | FDASISQVNEKINQSLA8IRKSDEXLHNV* | 22 |
| FDASISQVNEKINQSLAFX₁RKSDELX₂HNV* | 89 | FDASISQVNEKINQSLAF8RKSDELXHNV* | 23 |
| FDASISQVNEKINQSLAFIX₁KSDELLX₂NV* | 90 | FDASISQVNEKINQSLAFI8KSDELLXNV* | 24 |
| FDASISQVNEKINQSLAFIRX₁SDELLHX₂V* | 91 | FDASISQVNEKINQSLAFIR8SDELLHXV* | 25 |
| FDASISQVNEKINQSLAFIRKX₁DELLHNX₂* | 92 | FDASISQVNEKINQSLAFIRK8DELLHNX* | 26 |

In Table 3, with respect to SEQ ID NOs: 71-92, “X₁″=α, α-disubstituted non-natural amino acids with olefinic side chain cross-linked to X₂; “X₂″=α, α-disubstituted non-natural amino acids with olefinic side chain cross-linked to X₁, and * comprises Z-PEG(n)-cholesterol or Z-PEG(n)-thiocholesterol, wherein n=1-36, and wherein in Z is a diamino acid (e.g., lysine or ornithine). In Table 3, with respect to SEQ ID NOs: 5-26, “8″=(R)-α-(7′-octenyl)alanine; “X”=(S)-α-(4′-pentenyl)alanine, and * =Lys(epsilon-(PEG)4-thiocholesterol). In some instances of SEQ ID NOs:71-92 or 5-26, the * is Lys(epsilon-(PEG)4-cholesterol) or Lys(epsilon-(PEG)4-thiocholesterol), wherein n=1-36. In some instances of SEQ ID NOs: 71-92, the * =the one of the Formula II or III below, wherein n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36). In some instances of SEQ ID NOs: 71-92, the * =the one of Formula II or III below, wherein n=16. In some instances of SEQ ID NOs: 71-92, the * =the one Formula II or III below, wherein n=20. The two formulae indicated by the “*” include:

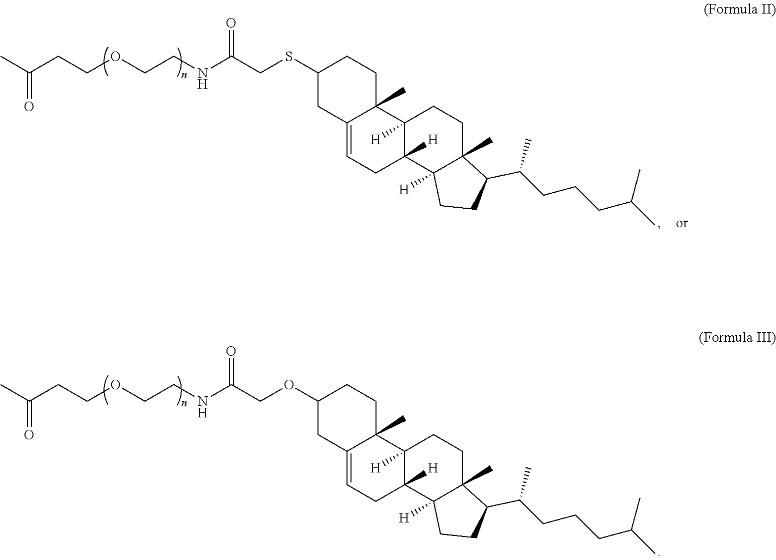

In some instances of SEQ ID NOs:71-92, the * =Formula II, wherein n=1-36 (e.g., n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36). In some instances of SEQ ID NOs:71-92, the * =Formula II, wherein n=16. In some instances of SEQ ID NOs:71-92, the * =Formula II, wherein n=20. In some instances of SEQ ID NOs:71-92, the * =Formula III, wherein n=1-36 (e.g., n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36). In some instances of SEQ ID NOs:71-92, the * =Formula III, wherein n=16. In some instances of SEQ ID NOs:71-92, the * =Formula III, wherein n=20. In some instances of SEQ ID NOs:5-26, the * =the one of the Formula IV or V below, wherein n=1-36 (n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36). In some instances of SEQ ID NOs: 5-26, the * =the one of Formula IV or V below, wherein n=16. In some instances of SEQ ID NOs: 5-26, the * =the one Formula IV or V below, wherein n=20. The two formulae indicated by the “*” include:

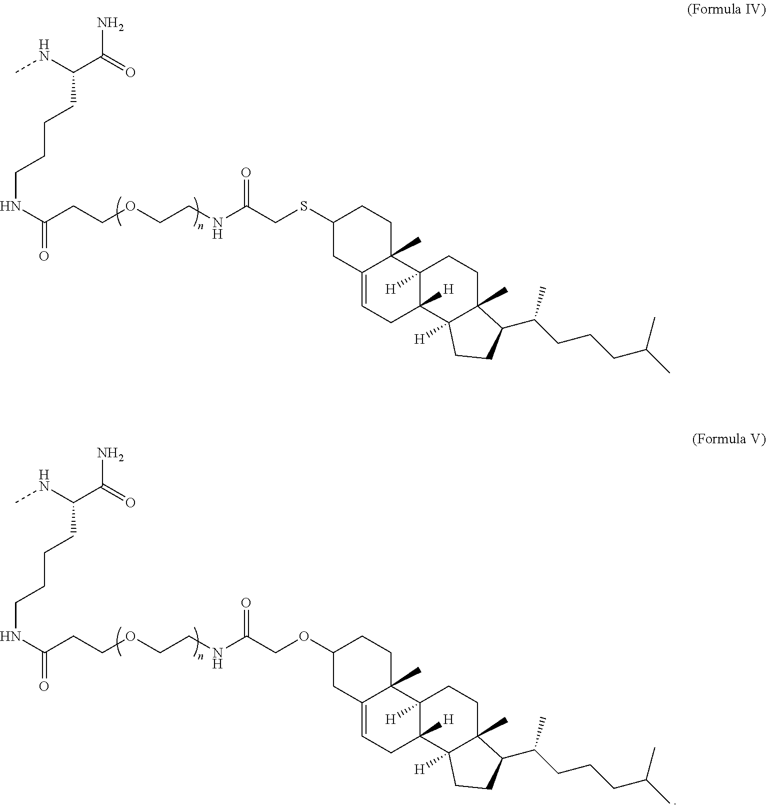

In some instances of SEQ ID NOs:5-26, the * =Formula IV, wherein n=1-36 (e.g., n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36). In some instances of SEQ ID NOs: 5-26, the * =Formula IV, wherein n=16. In some instances of SEQ ID NOs: 5-26, the * =Formula IV, wherein n=20. In some instances of SEQ ID NOs: 5-26, the * =Formula V, wherein n=1-36 (e.g., n=1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36). In some instances of SEQ ID NOs: 5-26, the * =Formula V, wherein n=16. In some instances of SEQ ID NOs: 5-26, the * =Formula V, wherein n=20. It should be understood that the above conjugates can be modified to include additional amino acids (e.g., 1, 2, 3, 4, 5, 6, 7, 8, 9, 10 amino acids added) at the N- and/or C-terminus, and/or to have N- and/or C-terminal deletions (e.g., 1, 2, 3, 4, 5, 6, 7, 8, 9, 10 amino acids deleted). In some instances, the conjugates are derived from SEQ ID NO: 100 (e.g. comprise or consist of the amino acid sequence of SEQ ID NO: 100 or an internally cross-linked version thereof or a variant thereof). In some instances, the conjugate does not comprise the amino acid sequence NAGKST (SEQ ID NO:258). In some instances, the conjugate does not comprise the amino acid sequence NAGKS (SEQ ID NO:259). In some instances, the conjugate does not comprise the amino acid sequence NAGK (SEQ ID NO:260). In some instances, the conjugate does not comprise the amino acid sequence NAG. In some instances, the conjugate does not comprise the amino acid sequence NA. In some instances, the conjugate does not comprise the amino acids corresponding to positions 517-522, 517-521, 517-520, 517-519, or 517-518 of SEQ ID NO:1 (or the corresponding amino acids of an RSV-F protein of another strain (see, e.g., FIG. 4)). In some instances, the conjugate is 25 to 34 (e.g., 25, 26, 27, 28, 29, 30, 31, 32, 33, or 34) amino acids in length. In some instances, the conjugate is 30 amino acids in length.

The disclosure encompasses each and structurally-stabilized peptide conjugate listed in Table 3 as well as variants thereof (e.g., having 1, 2, 3, 4, 5, 6, 7, 8, 9, 10, 11, 12, 13, 14, 15, 16) amino acid substitutions, insertions, and/or deletions, except at the positions of the staple, i.e., the bolded residues in Table 3, and except at the position of the lipidation (i.e., the * in Table 3)). In some instances, the variant has 1 to 10 amino acid substitutions, insertions, and/or deletions, except at the positions of the staple, (i.e., the bolded residues in Table 3), and except at the position of the lipidation (i.e., the * in Table 3)). In some instances, the variant has 1 to 5 amino acid substitutions, insertions, and/or deletions, except at the positions of the staple, (i.e., the bolded residues in Table 3), and except at the position of the lipidation (i.e., the * in Table 3)). In some instances, the variant has 1 to 3 amino acid substitutions, insertions, and/or deletions, except at the positions of the staple (i.e., the bolded residues in Table 3), and except at the position of the lipidation (i.e., the * in Table 3)). In some instances, the variant has 1 to 10, 10 to 5, 1 to 3, 2, or 1 amino acid substitutions, insertions, and/or deletions, except at the positions of the staple (i.e., the bolded residues in Table 3), and except at the position of the lipidation (i.e., the * in Table 3)).

In some instances, the structurally-stabilized peptide conjugate is 25 to 34 (e.g., 25, 26, 27, 28, 29, 30, 31, 32, 33, or 34) amino acids in length. In some instances, the structurally-stabilized peptide conjugate is 25 to 30 (e.g., 25, 26, 27, 28, 29, or 30) amino acids in length. In some instances, the structurally-stabilized peptide conjugate is 30 amino acids in length. In some instances, the structurally-stabilized peptide conjugate described above has one or more (1, 2, 3, 4, 5) of the properties listed below: (i) are alpha-helical; (ii) are protease resistant; (iii) bind to an RSV 5-helix bundle protein; (iv) inhibits infection of a cell by RSV; and/or (v) prevents infection of a cell by RSV.

In some instances, the conjugate has at least 70%, at least 75%, at least 80%, at least 85%, at least 90%, or at least 95% sequence identity to one of the single-stapled peptides in Table 3 (wherein the variation in the sequence is not at the staple positions nor at lipidation position). It is understood that the variation is not at the staple position (i.e., the bolded residues in Table 3), nor at the site of lipidation (i.e., the * in Table 3). In some instances, the conjugate is 100% identical to a conjugate in Table 3. In some instances, the conjugate is 25 to 34 (e.g., 25, 26, 27, 28, 29, 30, 31, 32, 33, or 34) amino acids in length. In some instances, the conjugate is 25 to 30 (e.g., 25, 26, 27, 28, 29, or 30) amino acids in length. In some instances, the conjugate is 30 amino acids in length. In some instances, the conjugate described above has one or more (1, 2, 3, 4, 5) of the properties listed below: (i) are alpha-helical; (ii) are protease resistant; (iii) bind to an RSV 5-helix bundle protein; (iv) inhibits infection of a cell by RSV; and/or (v) prevents infection of a cell by RSV.

In some instances, any substitution described herein is a conservative substitution. In some instances, any substitution described herein is a non-conservative substitution.

### Pharmaceutical Compositions

One or more of any of the structurally-stabilized (e.g., stapled) peptides or structurally-stabilized (e.g., stapled) peptide conjugates described herein can be formulated for use as or in pharmaceutical compositions. The pharmaceutical compositions may be used in the methods of treatment or prevention described herein. In some instances, the pharmaceutical composition comprises a structurally-stabilized peptide described herein and a pharmaceutically acceptable carrier. In some instances, the pharmaceutical composition comprises a structurally-stabilized peptide conjugate described herein and a pharmaceutically acceptable carrier. In certain instances, the pharmaceutical composition comprises a structurally-stabilized (e.g., stapled) peptide or a structurally-stabilized (e.g., stapled) peptide conjugate comprising or consisting of an amino acid sequence that is identical to an amino acid sequence set forth in Table 1 or Table 3 or a construct set forth in Table 2. In certain instances, the pharmaceutical composition comprises a structurally-stabilized (e.g., stapled) peptide or a structurally-stabilized (e.g., stapled) peptide conjugate comprising an amino acid sequence that is identical to an amino acid sequence set forth in Table 1 or Table 3 or a construct set forth in Table 2, except that the peptide further comprises three N-terminal amino acids, X1X2X3, where X1 is any amino acid, optionally S; and X2 and X3 are any negatively charged amino acid. In certain instances, the pharmaceutical composition comprises a structurally-stabilized peptide conjugate comprising or consisting of the amino acid sequence of any one of SEQ ID NOs: 27, 29, 36, 43, and 46 or any one of SEQ ID NOs: 49, 51, 58, 65, and 68 and a pharmaceutically acceptable carrier. In certain instances, the pharmaceutical composition comprises a structurally-stabilized peptide comprising or consisting of the amino acid sequence of SEQ ID NO: 29 or SEQ ID NO:51 and a pharmaceutically acceptable carrier. In certain instances, the pharmaceutical composition comprises a structurally-stabilized peptide comprising or consisting of the amino acid sequence of SEQ ID NO: 43 or SEQ ID NO:65 and a pharmaceutically acceptable carrier. In certain instances, the pharmaceutical composition comprises a structurally-stabilized peptide comprising or consisting of the amino acid sequence of SEQ ID NO: 281 and a pharmaceutically acceptable carrier. In certain instances, the pharmaceutical composition comprises a structurally-stabilized peptide conjugate comprising or consisting of the amino acid sequence of any one of SEQ ID NOs: 71, 73, 80, 87, and 90 or any one of SEQ ID NOs: 5, 6, 14, 21, and 24 and a pharmaceutically acceptable carrier. In certain instances, the pharmaceutical composition comprises a structurally-stabilized peptide conjugate comprising or consisting of the amino acid sequence of SEQ ID NO: 5 or SEQ ID NO:73 and a pharmaceutically acceptable carrier. In certain instances, the pharmaceutical composition comprises a structurally-stabilized peptide conjugate comprising or consisting of the amino acid sequence of SEQ ID NO: 21 or SEQ ID NO:87 and a pharmaceutically acceptable carrier. In certain instances, the pharmaceutical composition comprises a structurally-stabilized (e.g., stapled) peptide or a structurally-stabilized (e.g., stapled) peptide conjugate comprising or consisting of an amino acid sequence that is identical to an amino acid sequence set forth in Table 1, Table 2, or Table 3, except for 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, or 1 amino acid substitution, insertion, or deletion. It will be appreciated that the amino acid substitution, insertion, or deletion is not at a stapling position (e.g., is not at position 1 or 8 of the amino acid sequence of SEQ ID NO:6). In certain instances, the pharmaceutical composition comprises a structurally-stabilized (e.g., stapled) peptide or a structurally-stabilized (e.g., stapled) peptide conjugate comprising or consisting of an amino acid sequence that is identical to the amino acid sequence of any one of SEQ ID NOs: 27, 29, 36, 43, 46, 49, 51, 58, 65, 68, 71, 73, 80, 87, 90, 5, 6, 14, 21, and 24 except for 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, or 1 amino acid substitution, insertion, or deletion. These changes to the amino acid sequences can be made on the non-interacting alpha-helical face of these peptides (i.e., to the amino acids that do not interact with the 5 helix bundle of RSV-F protein) and/or on the interacting alpha-helical face (i.e., to the amino acids that interact with the 5 helix bundle of RSV-F protein), so long as they are not at a stapling position (e.g., at positions 1 and 8 of the amino acid sequence of SEQ ID NO:6). Such compositions can be formulated or adapted for administration to a subject via any route, e.g., any route approved by the Food and Drug Administration (FDA). Exemplary methods are described in the FDA's CDER Data Standards Manual, version number 004 (which is available at fda.give/cder/dsm/DRG/drg00301.htm). For example, compositions can be formulated or adapted for administration by inhalation (e.g., oral and/or nasal inhalation (e.g., via nebulizer or spray)), injection (e.g., intravenously, intra-arterial, subdermally, intraperitoneally, intramuscularly, and/or subcutaneously); and/or for oral administration, transmucosal administration, and/or topical administration (including topical (e.g., nasal) sprays, eye drops, and/or solutions).

In some instances, the pharmaceutical compositions are formulated or adapted for administration by nasal spray/drop, nebulization, subcutaneous administration, intravenous administration. In some instances, the pharmaceutical compositions are formulated or adapted for administration by topical modes (e.g., nasal spray, nebulization).

In some instances, pharmaceutical compositions can include an effective amount of one or more structurally-stabilized (e.g., stapled) peptides or structurally-stabilized (e.g., stapled) peptide conjugates. The terms “effective amount” and “effective to treat,” as used herein, refer to an amount or a concentration of the described agent (e.g., the structurally-stabilized (e.g., stapled) peptide or structurally-stabilized (e.g., stapled) peptide conjugate) or a pharmaceutical composition described herein utilized for a period of time (including acute or chronic administration and periodic or continuous administration) that is effective within the context of its administration for causing an intended effect or physiological outcome (e.g., treatment of infection).

Pharmaceutical compositions of this disclosure can include one or more structurally-stabilized (e.g., stapled) peptides or structurally-stabilized (e.g., stapled) peptide conjugates described herein and any pharmaceutically acceptable carrier and/or vehicle. In some instances, pharmaceutical compositions can further include one or more additional therapeutic agents in amounts effective for achieving a modulation of disease or disease symptoms.

The term “pharmaceutically acceptable carrier or adjuvant” refers to a carrier or adjuvant that may be administered to a patient or a subject from another species provided herein, together with a compound of this disclosure (e.g., a structurally-stabilized (e.g., stapled) peptide or structurally-stabilized (e.g., stapled) peptide conjugate), and which does not destroy the pharmacological activity thereof and is nontoxic when administered in doses sufficient to deliver a therapeutic amount of the compound.

In some instances, the pharmaceutical compositions of this disclosure include one or more of acetate, citrate and/or maleate. In some instances, the pharmaceutical compositions can include water or phosphate buffer saline (PBS). In some instance, the pharmaceutical compositions can include chitosan.

The pharmaceutical compositions disclosed herein can include one or more pharmaceutically acceptable salts. In some instances, the pharmaceutically acceptable salts include salts comprising hydrochloride, sodium, sulfate, acetate, phosphate or diphosphate, chloride, potassium, maleate, calcium, citrate, mesylate, nitrate, tartrate, aluminum, gluconate, and any combination thereof.

The pharmaceutical compositions of this disclosure may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intra-cutaneous, intra-venous, intra-muscular, intra-articular, intra-arterial, intra-synovial, intra-sternal, intra-thecal, intra-lesional and intra-cranial injection or infusion techniques.

In some instances, one or more structurally-stabilized (e.g., stapled) peptide or structurally-stabilized (e.g., stapled) peptide conjugate disclosed herein can be further conjugated, for example, to a carrier protein. Such conjugated compositions can be monovalent or multivalent. For example, conjugated compositions can include one structurally-stabilized (e.g., stapled) peptide conjugate disclosed herein conjugated to a carrier protein. Alternatively, as another example, conjugated compositions can include two or more structurally-stabilized (e.g., stapled) peptide conjugates disclosed herein further conjugated to a carrier.

When two entities are “conjugated” to one another, they are linked by a direct or indirect covalent or non-covalent interaction. In certain instances, the association is covalent. In other instances, the association is non-covalent. Non-covalent interactions include hydrogen bonding, van der Waals interactions, hydrophobic interactions, magnetic interactions, electrostatic interactions, etc. An indirect covalent interaction occurs when two entities are covalently connected, optionally through a linker group.

Carrier proteins can include any protein that increases or enhances stability, half-life, tissue exposure, and/or immunogenicity in a subject. Exemplary carrier proteins are described in the art (see, e.g., Fattom et al., Infect. Immun., 58:2309-2312, 1990; Devi et al., Proc. Natl. Acad. Sci. USA 88:7175-7179, 1991; Li et al., Infect. Immun. 57:3823-3827, 1989; Szu et al., Infect. Immun. 59:4555-4561, 1991; Szu et al., J. Exp. Med. 166:1510-1524, 1987; and Szu et al., Infect. Immun. 62:4440-4444, 1994). Polymeric carriers can be a natural or a synthetic material containing one or more primary and/or secondary amino groups, azido groups, or carboxyl groups. Carriers can be water soluble.

Methods of Making Stapled or Stitched Peptides Derivatized with PEG(n)-Thiocholesterol or PEG(n)-Cholesterol Moieties

In one aspect this disclosure features a method of making a structurally-stabilized peptide derivatized with PEG(n)-thiocholesterol or PEG(n)-cholesterol Moieties. The fully on-resin synthetic method involves (a) providing a peptide comprising at least two non-natural amino acids with olefinic side chains (e.g., SEQ ID NO: 27-70), (b) cross-linking the peptide, in some instances by a ruthenium catalyzed metathesis reaction, and (c) derivatizing the C-terminus on resin with a PEG linker of variable length connected to a thiocholesterol or cholesterol moiety.

In some instances, the methods include cleaving the structurally-stabilized peptide from the resin. Cleaving a structurally-stabilized resin is known in the art. In some instances, cleaving occurs prior to derivatizing the C-terminus on resin with a PEG linker of variable length connected to a thiocholesterol or cholesterol moiety. See e.g., de Vries et al., Science, 2021 Mar 26;371(6536):1379-138, and Figueira et al., J. Virol. 91, e01554-16 (2016), each of which is incorporated by reference in its entirety. In other aspects, cleaving is performed after the step of derivatizing the C-terminus on resin with a PEG linker of variable length connected to a thiocholesterol or cholesterol moiety.

In instances in which the cleaving step is performed prior to the derivatizing step, the methods include use of a compound having one of the following formulae:

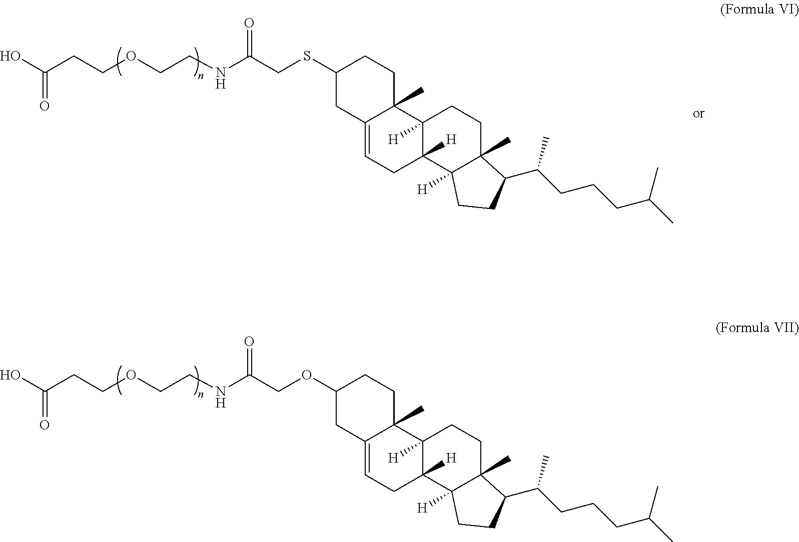

wherein n is 1-36. In some instances, n is 4, 5, 6, 7, 8, 10, 12, 14, 16, 18, 20, 22, 24, 26, 28, 30, 32, 34, or 36. Thus, also disclosed herein are compounds having one of the above formulae.

Stapled peptide synthesis: Fmoc-based solid-phase peptide synthesis was used to synthesize stapled peptide fusion inhibitors in accordance with our reported methods for generating all-hydrocarbon stapled peptides (Bird et al., Curr. Protocol. Chem, Biol., 3(3):99-117 (2011; Bird et al., Methods Enzymol., 446:369-86(2008). To achieve the various staple lengths, α-methyl, α-alkenyl amino acids were installed in specific pairings at discrete positions, such as for i, i+7 positioning the use of one S-pentenyl alanine residue (S5) and one R-octenyl alanine residue (R8). For the stapling reaction, Grubbs 1st generation ruthenium catalyst dissolved in dichloroethane was added to the resin-bound peptides. To ensure maximal conversion, three to five rounds of stapling were performed. After appending the PEG(n)-thiocholesterol or PEG(n)-cholesterol moiety (see below), the peptides were then cleaved off of the resin using trifluoroacetic acid, precipitated using a hexane:ether (1:1) mixture, air dried, and purified by LC-MS. All peptides were quantified by amino acid analysis.

Stitchedpeptide synthesis: Methods of synthesizing the stitched peptides described herein are known in the art. Nevertheless, the following exemplary method may be used. Synthetic chemistry transformations and protecting group methodologies (protection and deprotection) useful in synthesizing the compounds described herein are known in the art and include, for example, those such as described in Bird et al., ACS Chem Biol. (2020) 15(6):1340-1348; Hilinski et al., J Am Chem Soc. (2014) 136(35):12314-22; R. Larock, Comprehensive Organic Transformations, VCH Publishers (1989); T. W. Greene and P. G. M. Wuts, Protective Groups in Organic Synthesis, 3d. Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

C-terminal derivatization of stapled or stitched peptides with PEG(n)-thiocholesterol or PEG(n)-cholesterol using an on-resin synthetic approach: To generate the carboxy thiocholesterol or carboxy cholesterol reagent for peptide derivatization by solid phase synthesis, thiocholesterol was dissolved in dichloromethane (DCM) or cholesterol was dissolved in tetrahydrofuran (THF) at 0.1 M and added to a round bottom flask. 3 eq of a base (diisopropylethylamine for thiocholesterol or sodium hydride or potassium t-butoxide for cholesterol) was added with stirring. 5 eq of the t-butyl ester of bromoacetic acid was added next and the reaction was stirred for 2 hours at room temperature followed by 30 min at 40° C. Two volumes of trifluoroacetic acid (relative to solvent) was added and the reaction was stirred at room temperature for 30 min. The reaction progress was monitored by TLC (19:1 Hex:EtOAc for thiocholesterol and 3:1 Hex:EtOAc for cholesterol) with KMnO₄ staining. (Thio)cholesterol, for example, migrated with the solvent front with (thio)ether slowing migration by ˜20% and TFA hydrolysis brought the spot to baseline. The reaction mixture was added to 5 vol water and the 1 vol DCM was added. The solvent layer was washed with 0.1M HCl, brine and dried with sodium sulfate. Removal of the solvent by Rotovap yielded an orange heavy oil that was used without further purification. The yield was near quantitative. Purity was determined to be greater than 90% by NMR of the olefin proton vs the new CH₂ singlet. For peptide derivatization with thiocholesterol or cholesterol, the completed resin bound peptide sequence was treated with 20% piperidine/DMF followed by capping with acetic anhydride to block the N-terminal amine before the C-terminal side chain lysine amine was revealed by treatment with 2% hydrazine in DMF, 5× for 10 min each. The amine was acylated with an Fmoc-protected PEG(n) amino acid (e.g., n=1-36 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36)) at which point the olefins were crosslinked by treating with Grubbs(I) catalyst, 3× for 2 h each. Upon completion, the Fmoc was removed from the C-terminal NH of the PEG reagent and the amine was acylated with carboxy-thiocholesterol (or carboxy-cholesterol) for 30 min. TFA cleavage yielded a crude product of excellent purity that was further purified using semi-prep HPLC.

The peptide sequences of this invention can be made by chemical synthesis methods, which are well known to the ordinarily skilled artisan. See, for example, Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the α-NH₂ protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Insertion of a linking amino acid may be performed as described in, e.g., Young and Schultz, J Biol Chem. 2010 Apr. 9; 285(15): 11039-11044. Alternatively, the longer synthetic peptides can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimal for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from, e.g., Advanced Chemtech or Gyros Protein Technologies. Peptide bonds can be replaced, e.g., to increase physiological stability of the peptide, by: a retro-inverso bonds (C(O)—NH); a reduced amide bond (NH-CH₂); a thiomethylene bond (S—CH₂ or CH₂—S); an oxomethylene bond (O—CH₂ or CH₂—O); an ethylene bond (CH₂—CH₂); a thioamide bond (C(S)—NH); a trans-olefin bond (CH═CH); a fluoro substituted trans-olefin bond (CF═CH); a ketomethylene bond (C(O)—CHR) or CHR—C(O) wherein R is H or CH3; and a fluoro-ketomethylene bond (C(O)-CFR or CFR—C(O) wherein R is H or F or CH3.

The peptides can be further modified by: acetylation, amidation, biotinylation, cinnamoylation, farnesylation, fluoresceination, formylation, myristoylation, palmitoylation, and other lipidation, specifically including thiocholesterol or cholesterol modification using the on-resin method disclosed herein, phosphorylation (Ser, Tyr or Thr), stearoylation, succinylation and sulfurylation. As indicated above, peptides can be conjugated to or contain linker atoms or moieties of variable length, for example, polyethylene glycol (PEG) moieties of variable length; alkyl groups (e.g., Cl-C20 straight or branched alkyl groups); fatty acid radicals; and combinations thereof. a, α-Disubstituted non-natural amino acids containing olefinic side chains of varying length can be synthesized by known methods (Williams et al. J. Am. Chem. Soc., 113:9276, 1991; Schafmeister et al., J. Am. Chem Soc., 122:5891, 2000; and Bird et al., Methods Enzymol., 446:369, 2008; Bird et al, Current Protocols in Chemical Biology, 2011). In some instances the stitched peptide comprises a kinkage between i, i+4, and i+4 and i+8. Such stitched peptides can be made in the context of SEQ ID NO:100. In some instances, the amino acids forming the staple or stitch are (R)-2-(4′-pentenyl)Alanine, 2,2-bis(4-pentenyl)glycine, and (S)-2-(4′-pentenyl)Alanine at positions i, i+4, and i+8, respectively, of the stitch. In some instances for peptides where an i linked to i+7, i+7 linked to i+14 stitch is used (four turns of the helix stabilized): one R-octenyl alanine (e.g., (R)-α-(7′-octenyl)alanine), one bis-pentenyl glycine (e.g., α,α-Bis(4′-pentenyl)glycine), and one R-octenyl alanine (e.g., (R)-α-(7′-octenyl)alanine) is used. In some instances for peptides where an i linked to i+7, i+7 linked to i+14 stitch is used (four turns of the helix stabilized ): one S-octenyl alanine (e.g., (S)-α-(7′-octenyl)alanine), one bis-pentenyl glycine (e.g., α,α-Bis(4′-pentenyl)glycine), and one R-octenyl alanine (e.g., (R)-α-(7′-octenyl)alanine) is used. In some instances for peptides where an i linked to i+7, i+7 linked to i+14 stitch is used (four turns of the helix stabilized): one S-octenyl alanine (e.g., (S)-α-(7′-octenyl)alanine), one bis-pentenyl glycine (e.g., α,α-Bis(4′-pentenyl)glycine), and one S-octenyl alanine (e.g., (S)-α-(7′-octenyl)alanine) is used. In some instances for peptides where an i linked to i+7, i+7 linked to i+14 stitch is used (four turns of the helix stabilized): one R-pentenyl alanine (e.g., (R)-α-(4′-pentenyl)alanine), one bis-octenyl glycine (e.g., α,α-Bis(7′-octenyl)glycine), and one S-pentenyl alanine (e.g., (S)-α-(4′-pentenyl)alanine) is used. In some instances for peptides where an i linked to i+7, i+7 linked to i+14 stitch is used (four turns of the helix stabilized): one R-pentenyl alanine (e.g., (R)-α-(4′-pentenyl)alanine), one bis-octenyl glycine (e.g., α,α-Bis(7′-octenyl)glycine), and one R-pentenyl alanine (e.g., (R)-α-(4′-pentenyl)alanine) is used. In some instances for peptides where an i linked to i+7, i+7 linked to i+14 stitch is used (four turns of the helix stabilized): one S-pentenyl alanine (e.g., (S)-α-(4′-pentenyl)alanine), one bis-octenyl glycine (e.g., α,α-Bis(7′-octenyl)glycine), and one R-pentenyl alanine (e.g., (R)-α-(4′-pentenyl)alanine) is used. In some instances for peptides where an i linked to i+7, i+7 linked to i+14 stitch is used (four turns of the helix stabilized): one S-pentenyl alanine (e.g., (S)-α-(4′-pentenyl)alanine), one bis-octenyl glycine (e.g., α,α-Bis(7′-octenyl)glycine), and one S-pentenyl alanine (e.g., (S)-α-(4′-pentenyl)alanine) is used. R-octenyl alanine is synthesized using the same route, except that the starting chiral auxiliary confers the R-alkyl-stereoisomer. Also, 8-iodooctene is used in place of 5-iodopentene. Inhibitors are synthesized on a solid support using solid-phase peptide synthesis (SPPS) on MBHA resin or Rink Amide AM resin (see, e.g., WO 2010/148335).

Fmoc-protected α-amino acids (other than the olefinic amino acids N-Fmoc-α,α-Bis(4′-pentenyl)glycine, (S)—N-Fmoc-α-(4′-pentenyl)alanine, (R)—N-Fmoc-α-(7′-octenyl)alanine, (R)—N-Fmoc-α-(7′-octenyl)alanine, and (R)—N-Fmoc-α-(4′-pentenyl)alanine), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, CA). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is reported in the art (Williams et al., Org. Synth., 80:31, 2003).

Again, methods suitable for obtaining (e.g., synthesizing), stitching, and purifying the peptides disclosed herein are also known in the art (see, e.g., Bird et. al., Methods inEnzymol., 446:369-386 (2008); Bird et al, Current Protocols in Chemical Biology, 2011; Walensky et al., Science, 305:1466-1470 (2004); Schafmeister et al., J. Am. Chem. Soc., 122:5891-5892 (2000); U.S. patent application Ser. No. 12/525,123, filed Mar. 18, 2010; and U.S. Pat. No. 7,723,468, issued May 25, 2010, each of which are hereby incorporated by reference in their entirety).

In some instances, the peptides are substantially free of non-stitched or non-stapled peptide contaminants or are isolated. Methods for purifying peptides include, for example, synthesizing the peptide on a solid-phase support. Following cyclization, multiple alternative solvent and purification schemes are known in the art for peptide and stapled peptide isolation and purification and may use solvents that include, but are not limited to, DMSO, DMSO/dichloromethane mixture, DMSO/NMP mixture, or a mixture/solution that does not include DMSO. The DMSO/dichloromethane or DMSO/NMP mixture may comprise about 30%, 40%, 50% or 60% DMSO. In a specific instance, a 50%/50% DMSO/NMP solution is used. The solution may be incubated for a period of 1, 6, 12 or 24 hours, following which the resin may be washed, for example with dichloromethane or NMP. In one instance, the resin is washed with NMP. Shaking and bubbling an inert gas into the solution may be performed.

Properties of the stitched or stapled peptides derivatized with a C-terminal PEG(n)-thiocholesterol or PEG(n)-cholesterol of the disclosure can be assayed, for example, using the methods described below and in the Examples.

Assays to Determine Characteristics and Anti-RSV Activity of Stapled HR2 Peptides Derivatized with PEG(n)-Thiocholesterol or PEG(n)-Cholesterol Moieties

Assays to Determine α-Helicity: Compounds are dissolved in an aqueous solution (e.g. 5 μM potassium phosphate solution at pH 7, or distilled H₂O, to concentrations of 25-50 μM). Circular dichroism (CD) spectra are obtained on a spectropolarimeter (e.g., Jasco J-710, Aviv) using standard measurement parameters (e.g. temperature, 20° C.; wavelength, 190-260 nm; step resolution, 0.5 nm; speed, 20 nm/see; accumulations, 10; response, I see; bandwidth, I nm; path length, 0.1 cm). The α-helical content of each peptide is calculated by dividing the mean residue ellipticity by the reported value for a model helical decapeptide (Yang et al., Methods Enzymol., 1986).

Assays to Determine Melting Temperature (Tm): Cross-linked or the unmodified template peptides are dissolved in distilled H₂O or other buffer or solvent (e.g. at a final concentration of 50 M) and Tm is determined by measuring the change in ellipticity over a temperature range (e.g. 4 to 95° C.) on a spectropolarimeter (e.g., Jasco J-710, Aviv) using standard parameters (e.g. wavelength 222 nm; step resolution, 0.5 nm; speed, 20 nm/see; accumulations, 10; response, 1 sec; bandwidth, 1 nm; temperature increase rate: 1° C./min; path length, 0.1 cm).

In Vitro Protease Resistance Assays: The amide bond of the peptide backbone is susceptible to hydrolysis by proteases, thereby rendering peptidic compounds vulnerable to rapid degradation in vivo. Peptide helix formation, however, typically buries and/or twists and/or shields the amide backbone and therefore may prevent or substantially retard proteolytic cleavage. The peptidomimetic macrocycles of the present invention may be subjected to in vitro enzymatic proteolysis (e.g. trypsin, chymotrypsin, pepsin) to assess for any change in degradation rate compared to a corresponding uncrosslinked or alternatively stapled polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 nm. Briefly, the peptidomimetic macrocycle and peptidomimetic precursor (5 mcg) are incubated with trypsin agarose (Pierce) (S/E ˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of In[S] versus time.

Peptidomimetic macrocycles and/or a corresponding uncrosslinked polypeptide can be each incubated with fresh mouse, rat and/or human serum (e.g. 1-2 mL) at 37° C. for, e.g., 0, 1, 2, 4, 8, and 24 hours. Samples of differing macrocycle concentration may be prepared by serial dilution with serum. To determine the level of intact compound, the following procedure may be used: The samples are extracted, for example, by transferring 100 μL of sera to 2 ml centrifuge tubes followed by the addition of 10 μL of 50% formic acid and 500 μL acetonitrile and centrifugation at 14,000 RPM for 10 min at 4+/−2° C. The supernatants are then transferred to fresh 2 ml tubes and evaporated on Turbovap under N₂<10 psi, 37° C. The samples are reconstituted in 100 μL of 50:50 acetonitrile:water and submitted to LC-MS/MS analysis. Equivalent or similar procedures for testing ex vivo stability are known and may be used to determine stability of macrocycles in serum.

Plasma Stability Assay: Stapled peptide stability can be tested in freshly drawn mouse plasma collected in lithium heparin tubes. Triplicate incubations are set up with 500 μl of plasma spiked with 10 M of the individual peptides. Samples are gently shaken in an orbital shaker at 37° C. and 25 μl aliquots are removed at 0, 5, 15, 30, 60, 240, 360 and 480 min and added to 100 μl of a mixture containing 10% methanol:10% water:80% acetonitrile to stop further degradation of the peptides. The samples are allowed to sit on ice for the duration of the assay and then transferred to a MultiScreen Solvinert 0.45 μm low-binding hydrophilic PTFE plate (Millipore). The filtrate is directly analyzed by LC-MS/MS. The peptides are detected as double or triple charged ions using a Sciex 5500 mass spectrometer. The percentage of remaining peptide is determined by the decrease in chromatographic peak area and log transformed to calculate the half-life.

In Vivo Protease Resistance Assays: A key benefit of peptide stapling is the translation of in vitro protease resistance into markedly improved pharmacokinetics in vivo. Liquid chromatography/mass spectrometry-based analytical assays are used to detect and quantitate stapled peptide levels in plasma. For pharmacokinetic analysis, peptides are dissolved in sterile aqueous 5% dextrose (1 mg/mL) and administered to C57BL/6 mice (Jackson Laboratory) by bolus tail vein or intraperitoneal injection (e.g. 5, 10, 25, 50 mg/kg). Blood is collected by retro-orbital puncture at 5, 30, 60, 120, and 240 minutes after dosing 5 animals at each time point. Plasma is harvested after centrifugation (2,500×g, 5 minutes, 4° C.) and stored at −70° C. until assayed. Peptide concentrations in plasma are determined by reversed-phase high performance liquid chromatography with electrospray ionization mass spectrometric detection (Aristoteli et al., Journal of Proteome Res., 2007; Walden et al., Analytical and Bioanalytical Chem., 2004). Study samples are assayed together with a series of 7 calibration standards of peptide in plasma at concentrations ranging from 1.0 to 50.0 μg/mL, drug-free plasma assayed with and without addition of an internal standard, and 3 quality control samples (e.g. 3.75, 15.0, and 45.0 μg/mL). Standard curves are constructed by plotting the analyte/internal standard chromatographic peak area ratio against the known drug concentration in each calibration standard. Linear least squares regression is performed with weighting in proportion to the reciprocal of the analyte concentration normalized to the number of calibration standards. Values of the slope and y-intercept of the best-fit line are used to calculate the drug concentration in study samples. Plasma concentration-time curves are analyzed by standard noncompartmental methods using WinNonlin Professional 5.0 software (Pharsight Corp., Cary, NC), yielding pharmacokinetic parameters such as initial and terminal phase plasma half-life, peak plasma levels, total plasma clearance, and apparent volume of distribution.

Persistence of stapled peptides of the invention in the nasal mucosa after topical administration (i.e. nose drops) and in the respiratory mucosa after intranasal application or nebulization is examined in the context of pre- and post-infection blockade of viral fusion and dissemination. Mice are exposed to single treatment by nose drop or nebulizer at a series of intervals preceding intranasal infection with RSV, and the duration of protection from mucosal infection (assessed histologically as described above or by PCR as describe below) is used to measure the relative mucosal stability and prophylactic efficacy of the stapled peptide constructs derivatized with PEG(n)-thiocholesterol or PEG(n)-cholesterol described herein.

In Vitro BindingAssays: To assess the binding and affinity of peptidomimetic macrocycles and peptidomimetic precursors to acceptor proteins, a fluorescence polarization assay (FPA) can be used, for example. The FPA technique measures the molecular orientation and mobility using polarized light and fluorescent tracer. When excited with polarized light, fluorescent tracers (e.g., FITC) attached to molecules or peptides and then bound to proteins of high apparent molecular weights (e.g. FITC-labeled peptides bound to a large protein) emit higher levels of polarized fluorescence due to their slower rates of rotation upon protein binding as compared to fluorescent tracers attached to smaller molecules or peptides alone (e.g. FITC-labeled peptides that are free in solution).

In Vitro Displacement Assays to Characterize Antagonists of Peptide-Protein Interactions: To assess the binding and affinity of compounds that antagonize the interaction between a peptide and an acceptor protein, a fluorescence polarization assay (FPA) utilizing a fluoresceinated peptide or peptidomimetic macrocycle derived from a template peptide sequence is used, for example. The FPA technique measures the molecular orientation and mobility using polarized light and fluorescent tracer. When excited with polarized light, fluorescent tracers (e.g., FITC) attached to molecules that are then bound to proteins with high apparent molecular weights (e.g. FITC-labeled peptides bound to a large protein) emit higher levels of polarized fluorescence due to their slower rates of rotation as compared to the FITC-derivatized molecules alone (e.g. FITC-labeled peptides that are free in solution). Compounds such as unlabeled stapled peptides and their conjugates that antagonize the interaction between the fluoresceinated peptide and an acceptor protein will be detected in a competitive binding FPA experiment and the differential potency of compounds in disrupting the interaction can be quantified and compared.

Five helix bundle protein production and fluorescence polarization assay: A C-terminal Hexa-His tagged (SEQ ID NO: 279) recombinant 5-helix bundle (5HB) protein is designed containing 5 of the 6 helices that comprise the core of the RSV-F trimer of hairpins, connected by short peptide linkers in accordance with the design of the gp41 5-HB (Root et al. Science, 291(5505):884-8 (2001); Bird et al., J Clin Invest. 2014 May; 124(5):2113-24). The plasmid is transformed into Escherichia coli BL21 (DE3), cultured in Luria broth, and induced with 0.1 M isopropyl 3-D-thiogalactoside overnight at 37° C. The cells are harvested by centrifugation for 20 minutes at 5,000 g, resuspended in buffer A (100 mM NaH2PO4, 20 mM Tris, 8 M urea; pH 7.4), and lysed by agitation at 4° C. overnight. The mixture is clarified by centrifugation (35,000 g for 30 minutes) before binding to a nickel-nitrilotriacetate (Ni-NTA) agarose (Qiagen) column at room temperature. The bound 5-HB is washed with buffer A (pH 6.3), eluted with buffer A (pH 4.5), renatured by diluting (1:2) with PBS (50 mM sodium phosphate, 100 mM NaCl; pH 7.5), and concentrated in a 10-kDa Amicon centricon (diluting and reconcentrating 7 times), yielding approximately 1 mg/ml protein solution. Purity of the protein is assessed by SDS-PAGE and determined to be >90%. Fluoresceinated derivatives of the peptides of the invention (25 nM) are incubated with 5-HB protein at the indicated concentrations in room temperature binding buffer (50 mM sodium phosphate, 100 mM NaCl; pH 7.5). Direct binding activity at equilibrium (e.g. 10 minutes) is measured by fluorescence polarization using a SpectraMax M5 microplate reader (BMG Labtech). For a competitive binding assay, a fixed concentration of FITC-peptide and 5-HB protein reflecting the EC90 for direct binding is then incubated with a serial dilution of acetylated SAH-RSV peptides to generate competition curves for comparative analyses. Binding assays are run in triplicate, and K is are calculated by nonlinear regression analysis of the competition binding isotherms using Prism software (GraphPad).

Assay to screen for binding activity to the RSV 5 helix bundle: In some instances, the methods disclosed herein include direct and competitive screening assays. For example, methods can include determining whether an agent alters (e.g., reduces) binding of one or more of the peptides and conjugates thereof disclosed herein to RSV (e.g., to RSV 5-helix bundle). In some instances, methods include (i) determining a level of binding between one or more of the peptides and conjugates thereof disclosed herein and RSV (e.g., to RSV 5-helix bundle) (e.g., in the absence of an agent); and (ii) detecting the level of binding between one or more peptides (e.g., the one or more peptides of (i)) and RSV (e.g., to RSV 5-helix bundle) in the presence of an agent, wherein a change (e.g., reduction) in the level of binding between the one or more peptides and RSV (e.g., to RSV 5-helix bundle) indicates that the agent is a candidate agent that binds to RSV; and (iii) selecting the candidate agent. In some instances, step (i) includes contacting one or more peptides with RSV (e.g., to RSV 5-helix bundle) and detecting the level of binding between one or more peptides with RSV (e.g., to RSV 5-helix bundle). In some instances, step (ii) includes contacting the one or more peptides and the agent with RSV (e.g., to RSV 5-helix bundle) and detecting the level of binding between one or more peptides with RSV (e.g., to RSV 5-helix bundle). RSV (e.g., to RSV 5-helix bundle) can be contacted with the one or more peptides and the agent at the same time or at different times (e.g., the one or more peptides can be contacted with RSV (e.g., to RSV 5-helix bundle) before or after the agent). In some embodiments, candidate agents are administered to a suitable animal model (e.g., an animal model of RSV) to determine if the agent reduces a level of RSV infection in the animal.

In some instances, one or both of the peptide and the RSV helix bundle can include a label, allowing detection of the peptide and/or the RSV helix bundle. In some instances, the peptide includes a label. In some instances, the RSV helix bundle includes a label. In some instances, both the peptide and the RSV helix bundle include a label. A label can be any label known in the art, including but not limited to a fluorescent label, a radioisotope label, or an enzymatic label. In some instances, the label is directly detectable by itself (e.g., radioisotope labels or fluorescent labels). In some instances, (e.g., in the case of an enzymatic label), the label is indirectly detectable, e.g., by catalyzing chemical alterations of a chemical substrate compound or composition, which chemical substrate compound or composition is directly detectable.

Conmpetitive RSV 5-HB binding assay by ELISA: Microwells are coated overnight at 4° C. with 50 μl of PBS containing neutravidin (4 μg/ml). Wells are washed twice with PBS containing 0.05% Tween 20 (PBS-T), and blocked with 4% BSA in PBS-T for 45 min at 37° C. Next, 50 1 of 250 nM biotinylated-PEG2-RSV HR2 (SEQ ID NO: 100) is added in PBS-T with 1% BSA and incubated with shaking for 1 hr followed by 4× washes with 300 μl of PBS-T. Then, a 1:2 serial dilution of stapled peptides of the invention starting at 10 μM containing 50 nM of recombinant 5-HB in 50 μL of PBS-T with 1% BSA is added to the plate and shaken at room temperature for 2 hr followed by 4× washes with 300 μl of PBS-T. Finally, 50 μL of a 1:5000 dilution of goat polyclonal to 6× His tag-HRP (“HHHHHH” disclosed as SEQ ID NO: 279) conjugated is added. Following incubation at RT for 40 min, the wells are washed five times, and developed by adding 50 μl of tetramethylbenzidine (TMB) solution. After 20 min, wells containing TMB solution are stopped by adding 50 μl of H₂SO₄ (2 M), and the absorbance at 450 nm is read on a microplate reader (Molecular Devices). The concentration of competitor peptide corresponding to a half-maximal signal (IC50) is determined by interpolation of the resulting binding curve using Prism software (Graphpad). Each peptide competitor is tested in triplicate in at least two separate experiments.

Cellular Localization Assays: To measure the localization of peptides or crosslinked polypeptides on or in cells, intact cells are incubated with fluoresceinated crosslinked polypeptides derivatized with PEG(n)-thiocholesterol or PEG(n)-cholesterol (5 μM) for 4 hours in serum-free media or in media supplemented with human serum at 37° C., washed twice with media and incubated with trypsin (0.25%) for 10 min at 37° C. The cells are washed again and resuspended in PBS. Cellular fluorescence is analyzed, for example, by using either a FACSCalibur flow cytometer or Cellomics' KineticScan^{RTM}″ HCS Reader.

Antiviral Efficacy Assays: The efficiency of the peptide-cholesterol conjugates of the invention in preventing and treating infection of live human Respiratory Syncytial Virus with Green Fluorescent Protein (RSV-GFP1) are evaluated in monolayer cell cultures. A549 cells plated in 384-well format are treated for 30 min with a serial dilution of stapled peptides (e.g. 1-5 M starting dose or a fixed dose, e.g. 2 M), performed in quadruplicate, followed by addition of GFP-RSV live virus assay (0.75-1.25 μL of virus per well) and incubation for 48-72 hours. Infected cells are then washed with PBS. Hoechst 33342 (cell permeable nuclear dye) and DRAQ7 (cell impermeable nuclear dye) are added and the plate imaged on a Molecular Devices ImageXpress Micro Confocal Laser at 4× magnification. GFP (+) cells are counted and total GFP(+) cells or percent GFP(+) cells are plotted using Prism software (Graphpad). Cytotoxicity is determined by the ratio of DRAQ7 (+) Hoechst 33342 (+) to DRAQ7 (−) Hoechst 33342 (+) cells.

### Methods of Use

The disclosure features methods of using any of the structurally-stabilized (e.g., stapled) peptides or structurally-stabilized peptide conjugates (or pharmaceutical compositions comprising said structurally-stabilized peptides or structurally-stabilized peptide conjugates) described herein for treating or preventing an RSV infection in a subject (e.g., human) in need thereof. In some instances, the treating alleviates, inhibits, or ameliorates the infection from which the subject (e.g., human) is suffering. In some instances, the subject is an animal. In some instances, the subject is a mammal such as a non-primate (e.g., cow, pig, horse, cat, dog, rat, etc.) or a primate (e.g., monkey or human). In some instances, the subject is a domesticated animal (e.g., a dog or cat). In some instances, the subject is a human. In certain instances, such terms refer to a non-human animal (e.g., a non-human animal such as a pig, horse, cow, cat or dog). In some instances, such terms refer to a pet or farm animal. In some instances, such terms refer to a human.

The structurally-stabilized (e.g., stapled) peptides or structurally-stabilized peptide- conjugates (or pharmaceutical compositions comprising the same) described herein are useful for treating a subject (e.g., human) having an RSV infection. In some instances, the structurally-stabilized peptide (or a pharmaceutical composition comprising the same) is used in treatment of an RSV infection in a subject (e.g., human). In some instances, the structurally-stabilized peptide conjugate (or a pharmaceutical composition comprising the same) is used in treatment of an RSV infection in a subject (e.g., human). In some instances, the subject is a human.

The structurally-stabilized (e.g., stapled) peptides or structurally-stabilized peptide- conjugates (or pharmaceutical compositions comprising the same) described herein are useful for preventing a subject (e.g., human) from having an RSV infection. In some instances, the structurally-stabilized peptide (or a pharmaceutical composition comprising the same) is used in prevention of an RSV infection in a subject (e.g., human). In some instances, the structurally-stabilized peptide conjugate (or a pharmaceutical composition comprising the same) is used in prevention of an RSV infection in a subject (e.g., human). In some instances, the subject is a human.

Thus, provided herein is a method of treating an RSV infection in a subject (e.g., a human) in need thereof, the method comprising administering to the subject a therapeutically effective amount of a structurally-stabilized peptide described herein (or a pharmaceutical composition comprising the structurally-stabilized peptide).

Also provided herein is a method of preventing an RSV infection in a subject (e.g., a human) in need thereof, the method comprising administering to the subject a therapeutically effective amount of a structurally-stabilized peptide described herein (or a pharmaceutical composition comprising the structurally-stabilized peptide).

Thus, provided herein is a method of treating an RSV infection in a subject (e.g., a human) in need thereof, the method comprising administering to the subject a therapeutically effective amount of a structurally-stabilized peptide conjugate described herein (or a pharmaceutical composition comprising the structurally-stabilized peptide conjugate).

Also provided herein is a method of preventing an RSV infection in a subject (e.g., a human) in need thereof, the method comprising administering to the subject a therapeutically effective amount of a structurally-stabilized peptide conjugate described herein (or a pharmaceutical composition comprising the structurally-stabilized peptide conjugate).

In certain instances, the foregoing methods comprise administering to the subject (e.g., human) a peptide described in Table 1, or a variant thereof (e.g., having 1, 2, 3, 4, 5, 6, 7, 8, 9, 10, 11, 12, 13, 14, 15 substitutions, insertions, or deletions), a construct described in Table 2, or a variant thereof (e.g., having 1, 2, 3, 4, 5, 6, 7, 8, 9, 10, 11, 12, 13, 14, 15 substitutions, insertions, or deletions), or a conjugate described in Table 3, or a variant thereof (e.g., having 1, 2, 3, 4, 5, 6, 7, 8, 9, 10, 11, 12, 13, 14, 15 substitutions, insertions, or deletions). In some cases, the peptides or constructs of Tables 1-3, further comprise three N-terminal amino acids, X1X2X3, wherein X₁ is any amino acid, optionally S; and X2 and X3 are any negatively charged amino acid.

In certain instances, the method comprises administering to the subject (e.g., human) a structurally-stabilized peptide comprising or consisting of the amino acid sequence of any one of SEQ ID NOs: 27, 29, 36, 43, and 46 or any one of SEQ ID NOs: 49, 51, 58, 65, and 68 (or a pharmaceutical composition comprising the same). In certain instances, the method comprises administering to the subject (e.g., human) a structurally-stabilized peptide comprising or consisting of the amino acid sequence of SEQ ID NO: 29 or SEQ ID NO:51 (or a pharmaceutical composition comprising the same). In certain instances, the method comprises administering to the subject (e.g., human) a structurally-stabilized peptide comprising or consisting of the amino acid sequence of SEQ ID NO: 43 or SEQ ID NO:65 (or a pharmaceutical composition comprising the same).

In certain instances, the method comprises administering to the subject (e.g., human) a structurally-stabilized peptide conjugate comprising or consisting of the amino acid sequence of any one of SEQ ID NOs: 71, 73, 80, 87, and 90 or any one of SEQ ID NOs: 5, 6, 14, 21, and 24 (or a pharmaceutical composition comprising the same). In certain instances, the method comprises administering to the subject (e.g., human) a structurally-stabilized peptide conjugate comprising or consisting of the amino acid sequence of SEQ ID NO: 5 or 73 (or a pharmaceutical composition comprising the same). In certain instances, the method comprises administering to the subject (e.g., human) a structurally-stabilized peptide conjugate comprising or consisting of the amino acid sequence of SEQ ID NO: 21, 281, or 87 (or a pharmaceutical composition comprising the same).

In certain instances, the method comprises administering to the subject (e.g., human) a variant of a structurally-stabilized peptide comprising or consisting of the amino acid sequence of any one of SEQ ID NOs: 27, 29, 36, 43, and 46 or any one of SEQ ID NOs: 49, 51, 58, 65, and 68 (e.g., having 1 to 10, 1 to 5, 1 to 3, 2, or 1 amino acid substitutions, insertions, and/or deletions relative to the amino acid sequence set forth in any one of any one of SEQ ID NOs: 27, 29, 36, 43, and 46 or any one of SEQ ID NOs: 49, 51, 58, 65, and 68, respectively, wherein the amino acid substitution(s) and/or deletion(s) is/are not at the staple positions and wherein the structurally-stabilized peptide binds to an RSV 5-helix bundle protein and/or inhibits infection of a cell by RSV and/or prevents infection of a cell by RSV) (or a pharmaceutical composition comprising the same).

In certain instances, the method comprises administering to the subject (e.g., human) a variant of a structurally-stabilized peptide conjugate comprising or consisting of the amino acid sequence of any one of SEQ ID NOs: 71, 73, 80, 87, and 90 or any one of SEQ ID NOs: 5, 6, 14, 21, and 24 (e.g., having 1 to 10, 1 to 5, 1 to 3, 2, or 1 amino acid substitutions, insertions, and/or deletions relative to the amino acid sequence set forth in any one of any one of SEQ ID NOs: 71, 73, 80, 87, and 90 or any one of SEQ ID NOs: 5, 6, 14, 21, and 24, respectively, wherein the amino acid substitution(s) and/or deletion(s) is/are not at the staple positions and wherein the structurally-stabilized peptide conjugate binds to a RSV 5-helix bundle protein and/or inhibits infection of a cell by RSV and/or prevents infection of a cell by RSV) (or a pharmaceutical composition comprising the same).

In certain instances, the method comprises administering to the subject (e.g., human) a structurally-stabilized peptide described in the section “Structurally-Stabilized Peptides” above (or a pharmaceutical composition comprising the same). In certain instances, the method comprises administering to the subject (e.g., human) a structurally-stabilized peptide conjugate described in the section “Structurally-Stabilized Peptide Conjugates” above (or a pharmaceutical composition comprising the same). In some instances, the method comprises administering to the subject (e.g., human) a structurally-stabilized peptide or a structurally-stabilized peptide conjugate described in the figures or working examples (or a pharmaceutical composition comprising the same). In one instance, the methods described herein involve administering a peptide or conjugate comprising the amino acid sequence of SEQ ID NO:281.

In some instances, the subject (e.g., human) is infected with an RSV In some instances, the subject (e.g., human) is at risk of being infected with an RSV (e.g., has been exposed to a subject infected with an RSV). In some instances, the subject (e.g., human) is suspected of being infected with an RSV (e.g., has been exposed to a subject infected with an RSV and displays one or more symptoms of RSV). Methods for determining if a subject is infected with an RSV are known in the art.

In some instances, the methods of treating or preventing an RSV infection further include repeatedly administering to the subject (e.g., human) a therapeutically effective amount of the structurally-stabilized peptide or structurally stabilized peptide conjugate described herein (or a pharmaceutical composition comprising the same) as required for the treatment or prevention of the RSV infection. In some instances, the methods of treating or preventing an RSV infection further include testing the subject (e.g., human) to determine that the subject has an RSV infection and subsequently administering the therapeutically effective amount of the structurally-stabilized peptide or structurally stabilized peptide conjugate described herein (or a pharmaceutical composition comprising the same). A subject can be selected for treatment based on, e.g., determining that the subject is at risk to acquire or has an RSV infection.

The structurally-stabilized peptide or structurally stabilized peptide conjugate described herein (or a pharmaceutical composition comprising the same) can be administered orally, intranasally, intravenously, subcutaneously, intramuscularly, or topically, including skin, nasal, sinus, ocular, oropharynx, respiratory tree, and lung administration. In some instances, the administration is by a topical respiratory application which includes application to the nasal mucosa, sinus mucosa, oropharyngeal mucosa, or respiratory tree, including the lungs. In some instances, topical application includes application to the skin or eyes. In some instances, the conjugates described herein increase bioavailability, increase blood circulation, alter pharmacokinetics, decrease immunogenicity and/or decrease the needed frequency of administration.

Specific dosage and treatment regimens for any particular patient or subject will depend upon a variety of factors, including the activity of the specific compound employed, the age, body weight, general health status, sex, diet, time of administration, rate of excretion, drug combination, the severity and course of the disease, condition or symptoms, the patient's or subject's disposition to the disease, condition or symptoms, and the judgment of the treating physician or veterinarian.

An effective amount can be administered in one or more administrations, applications or dosages. A therapeutically effective amount of a therapeutic compound (e.g., structurally-stabilized peptide or structurally-stabilized peptide conjugate) (i.e., an effective dosage) depends on the therapeutic compounds selected. The compositions can be administered from one or more times per day to one or more times per week, including once every other day. The skilled artisan will appreciate that certain factors may influence the dosage and timing required to effectively treat a subject, including but not limited to the risk to acquire or severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, effective amounts can be administered at least once.

Also provided herein is use of a structurally-stabilized peptide or structurally-stabilized peptide conjugate described herein (or a pharmaceutical composition comprising the same) in the manufacture of a medicament for treating an RSV infection in a subject (e.g., human).

Also provided herein is use of a structurally-stabilized peptide or structurally-stabilized peptide conjugate described herein (or a pharmaceutical composition comprising the same) in the manufacture of a medicament for preventing an RSV infection in a subject (e.g., human).

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Design and Synthesis of Stapled Lipopeptides of the RSV HR2 Domain to Block RSV Infection by Inhibiting Viral Fusion with the Host Membrane

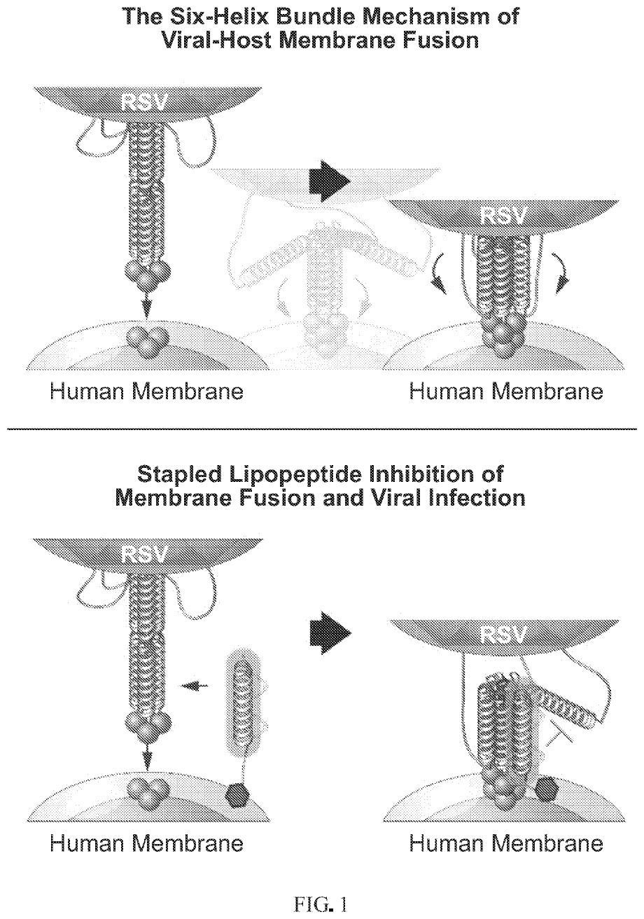

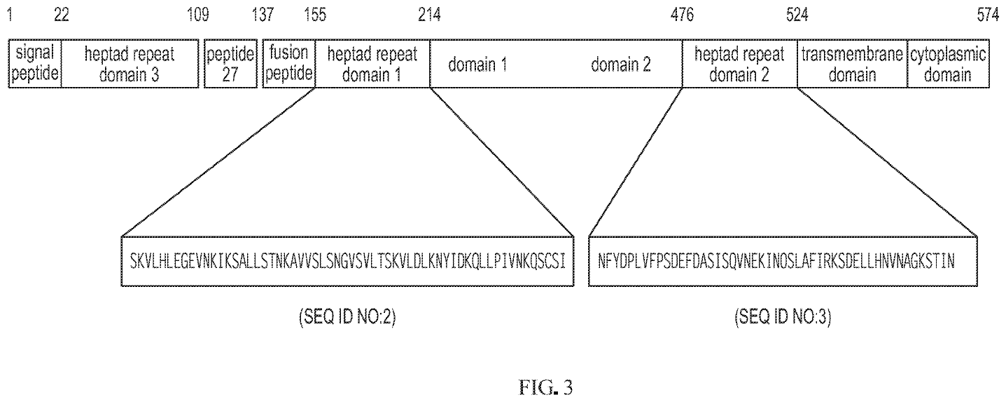

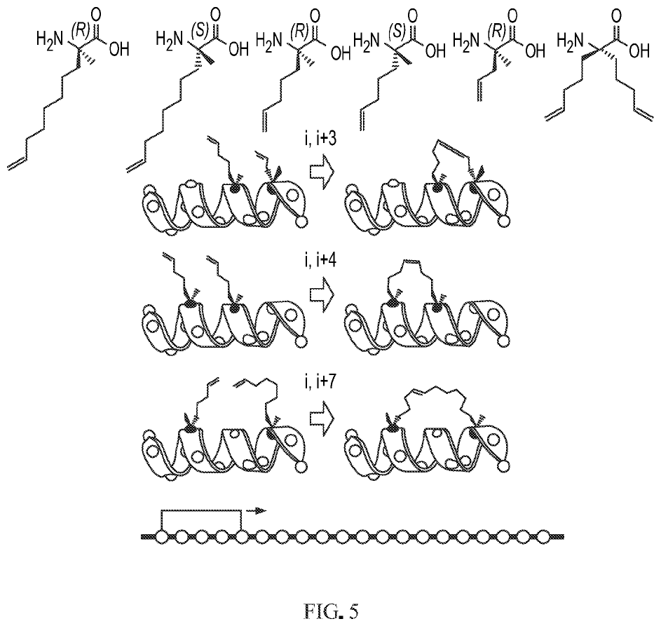

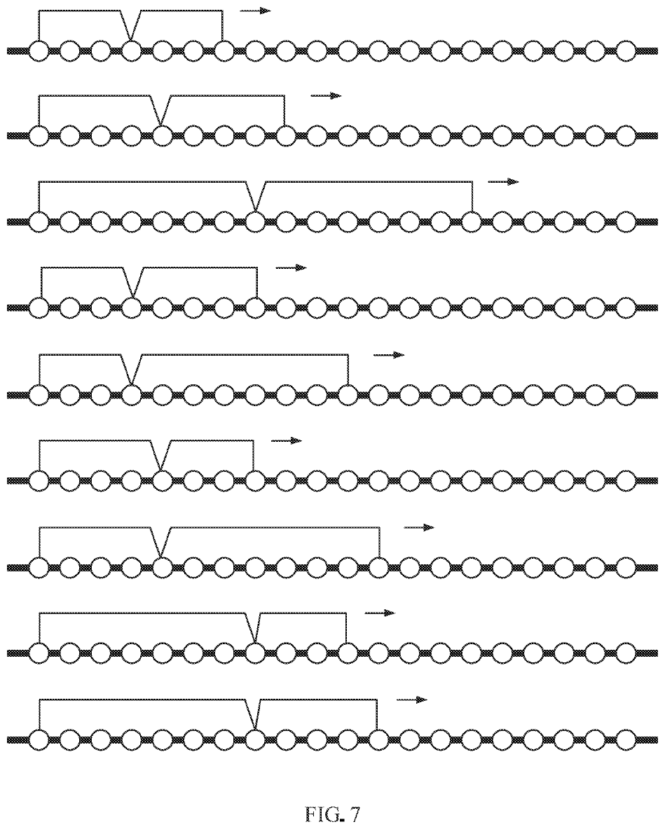

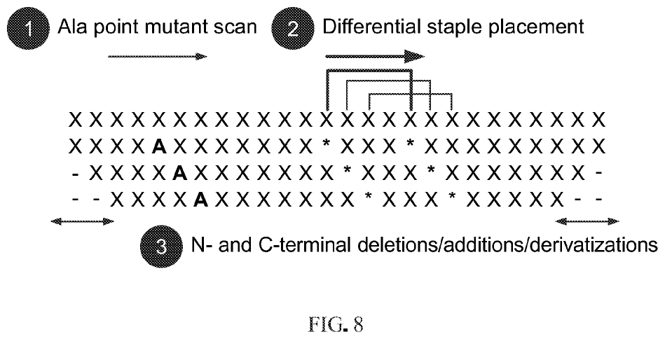

To design peptides that potently block the fusion of RSV to a host cell (FIG. 1), a series of stapled peptides bearing differentially localized chemical staples and derivatized with PEG(n)-thiocholesterol or PEG(n)-cholesterol moieties at the C-termini were designed. The designed peptides are synthesized on resin by solid phase synthesis. The differentially localized chemical staples are located within the RSV 1R₂ domain (i.e., amino acids 488-516 of SEQ ID NO:1) of the sequence of the surface (F) glycoprotein of the human RSV (see, FIG. 2-4) by replacing native residues with α, α-disubstituted non-natural olefinic residues (e.g., “X” for (S)-α-(4′-pentenyl)alanine and “8″ for (R)-α-(7′-octenyl)alanine installed at select i, i+7 positions or “X” for (S)-α-(4′-pentenyl)alanine installed at each of select i, i+4 positions) and combinations thereof in the form of double staples or stitches, followed by ruthenium-catalyzed olefin metathesis (see, FIGS. 5-7). The approach to designing, synthesizing, and identifying optimal stapled peptide constructs to target the RSV fusion apparatus includes the generation of Ala scan (e.g. mutants), staple scan, and variable N- and C-terminal deletion, addition, and derivatization libraries for conjugation to PEG-thiocholesterol or PEG-cholesterol moieties (see, FIG. 8). Stapled RSV HR2 peptides are constructed by replacing two naturally occurring amino acids with the non-natural (R)-2-(((9H-fluoren-9-yl)methoxy)carbonylamino)-2-methyl-dec-9-enoic acid/(R)-α-(7′-octenyl)alanine /(Fmoc-R8) and (S)-2-(((9H-fluoren-9-yl)methoxy)carbonylamino)-2-methyl-hept-6-enoic acid/(S)-α-(4′-pentenyl)alanine /(Fmoc-S5) amino acids at i, i+7 positions (i.e. flanking 7 amino acids) to generate a staple spanning two α-helical turns, or with two S5 non-natural amino acids at i, i+4 positions to generate a staple spanning one α-helical turn. Asymmetric syntheses of α, α-disubstituted amino acids are performed as previously described in detail (Schafmeister et al., J. Am. Chem. Soc., 2000; Walensky et al., Science, 2004; Bird et al. Current Protocols in Chemical Biology, 2011, each of which is incorporated by reference in its entirety).

### Example 2: Identifying optimally stapled RSV HR2 peptides bearing a C-terminal PEG4-thiocholesterol to achieve anti-viral activity in pseudotype and live virus assays

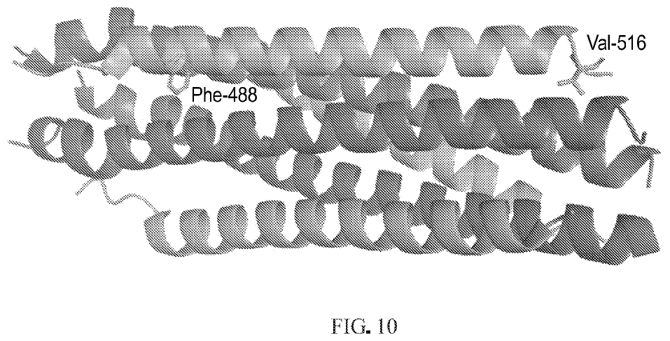

To enhance the potency and, thus, drug-like properties of RSV HR2 helices stabilized by insertion of an all-hydrocarbon staple alone, differentially stapled RSV HR2 constructs were further derivatized at the C-terminus with PEG(n)-thiocholesterol moieties. For example, the singly i, i+7 stapled peptide of SEQ TD NO: 4 blocked infection by the RSV virus with an IC50 of >5 μM. Thus, an iterative optimization campaign by “staple scanning” the alpha-helical region of the RSV HR2 domain spanning residues Phe 488 and Val 516 was initiated (see, FIG. 10).

Staple scanning was performed to respectively identify residues and binding surfaces critical for interaction, which dictates the design of optimized constructs and negative control mutants (see, FIG. 5). The peptide N-termini are capped with acetyl or a fluorophore (e.g. FITC, rhodamine), depending upon the experimental application. N-terminal acetyl-capped peptides were generated.

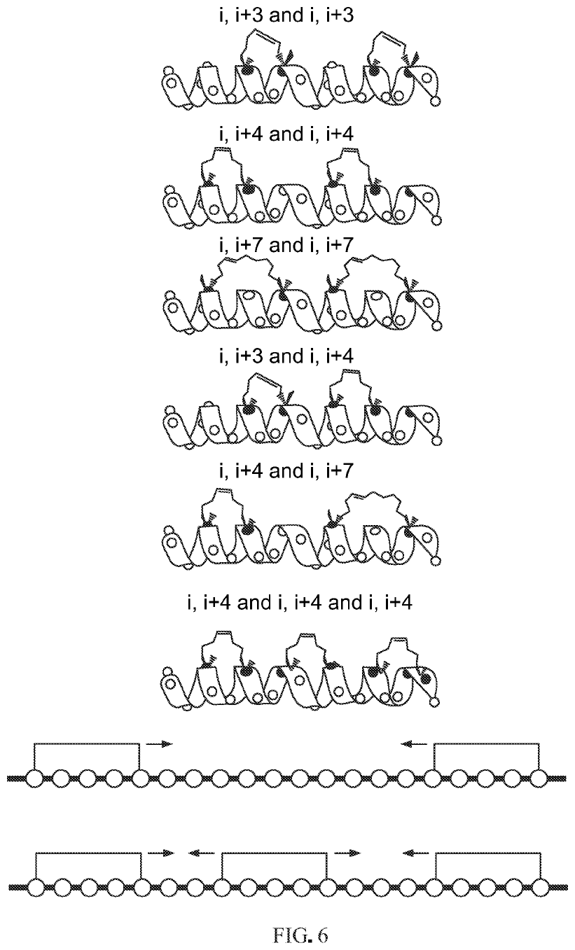

Doubly stapled peptides are generated by installing two-S5-S5, two R8-S5, or other combinations of crosslinking non-natural amino acids; multiply stapled or stitched peptides are generated using similar principles (see, FIG. 6).

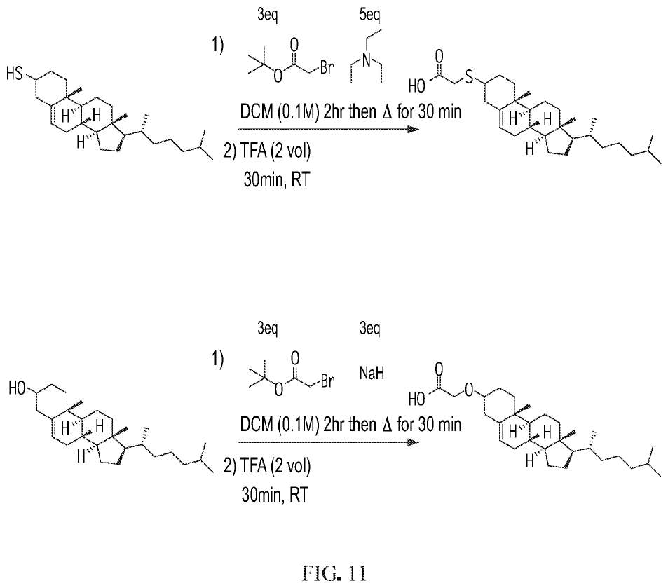

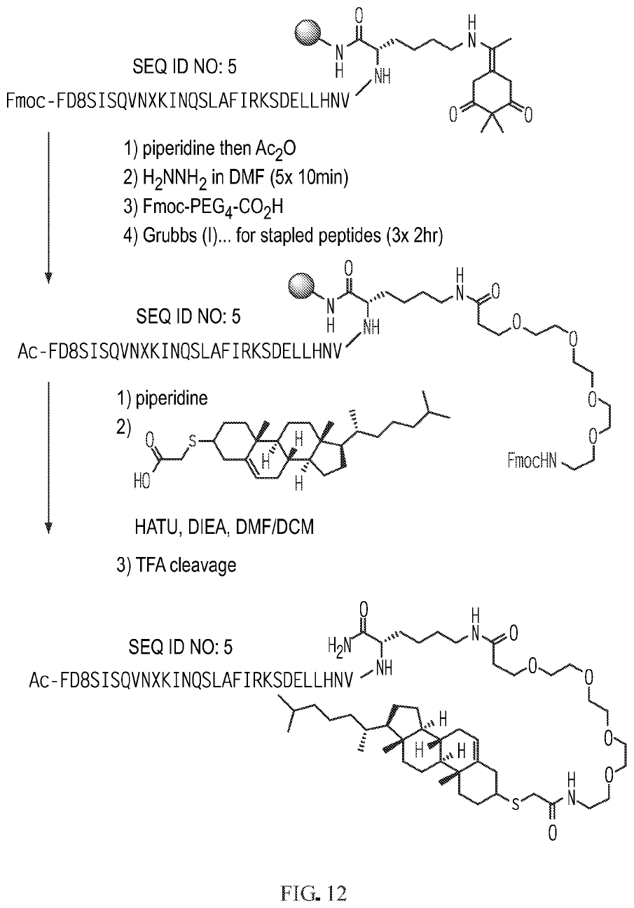

To enable peptide derivatization with thiocholesterol on resin, carboxy-thiocholesterol were synthesized according to the schematic presented in FIG. 11. To generated stapled lipopeptides of the RSV HR2 domain (see schema in FIG. 12), the completed resin-bound peptide (e.g., SEQ ID NO: 5) was capped with an acetyl group (by use of acetic anhydride) followed by deprotection of the C-terminal side chain lysine amine by treatment with 2% hydrazine. The amine was acylated with an Fmoc-protected PEG(n) amino acid (e.g., n=1-36) at which point the olefins were crosslinked by treating with Grubbs(T) catalyst. The Fmoc was removed from the C-terminal NH of the PEG(n) amino acid and the amine acylated with carboxy-thiocholesterol. The final peptide product was obtained after peptide deprotection and cleavage, and purification by reverse phase high performance liquid chromatography/mass spectrometry (LC/MS). The same procedure may be performed for derivatization with cholesterol, using cholesterol moieties instead of thiocholesterol moieties.

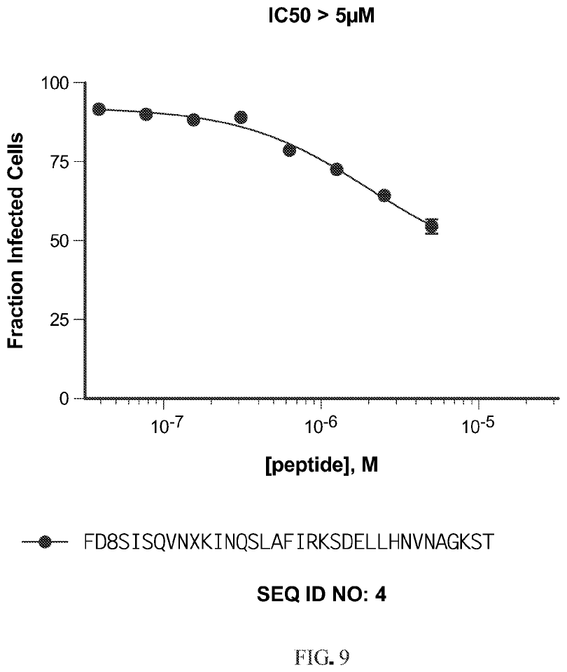

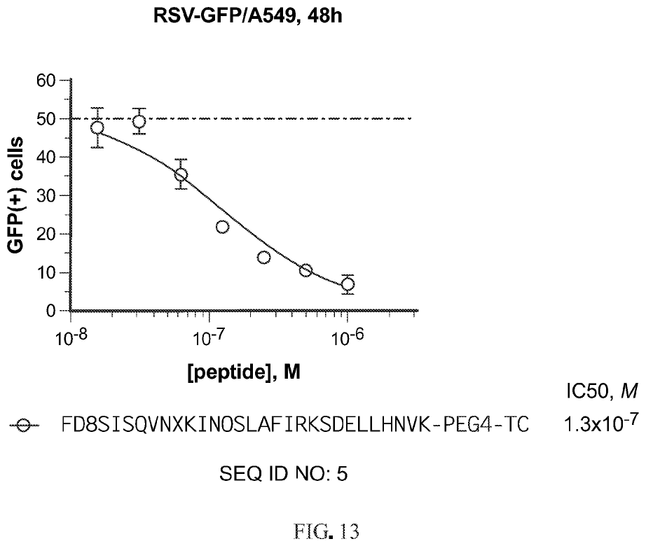

This chemical schema was applied to append a PEG4-thiocholesterol moiety to the C-terminus of the sequence set forth in SEQ ID NO: 51, thereby generating a conjugate having the sequence of SEQ ID NO:5. The resulting conjugate of SEQ ID NO:5 exhibited nanomolar antiviral activity (IC50=130 nM; see, FIG. 13) in an infectivity assay using live RSV virus, with an IC50 of 1.3×10⁻⁷M. Thus, removal of the 6 C-terminal residues of the sequence of SEQ ID NO:4 and the addition of a C-terminal PEG-thiocholesterol moiety improved the antiviral activity of the peptide by an order of magnitude (compare FIG. 9 to FIG. 13).

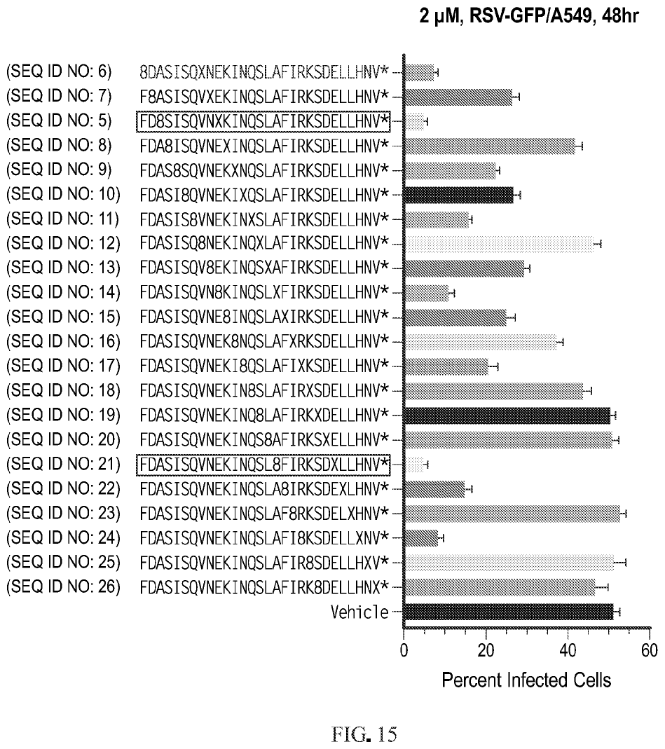

To identify the optimal location(s) for staple insertion, an exemplary i, i+7 staple scanning library of RSV HR2 (aa 488-516 of SEQ ID NO:1) derivatized with PEG(n)-thiocholesterol was generated by use of the synthetic schema depicted in FIGS. 11-12 (thereby generating SEQ ID NOs: 5-26). The differential antiviral activity of this library was evaluated in an RSV infectivity essay. Peptides having the sequences of SEQ ID NOs: 5, 6, 14, 21, and 24 exhibited potent activity among the various stapled RSV HR2 peptides in the GFP-RSV live virus assay (see, FIG. 15). Taken together, these data show that (1) appending a PEG4-thiocholesterol moiety using on-resin methodology to the C-terminus of a structurally-stabilized RSV HR2 peptide can endow potent antiviral activity among a panel of differentially i, i+7 stapled peptides with lesser or no activity (compare FIG. 9 and FIG. 13).

### Example 3: Determining the Optimal PEG Linker Length within Stapled RSV HR2 Peptides Bearing a C-Terminal PEG(n)-Thiocholesterol to Achieve Anti-Viral Activity in Pseudotype and Live Virus Assays

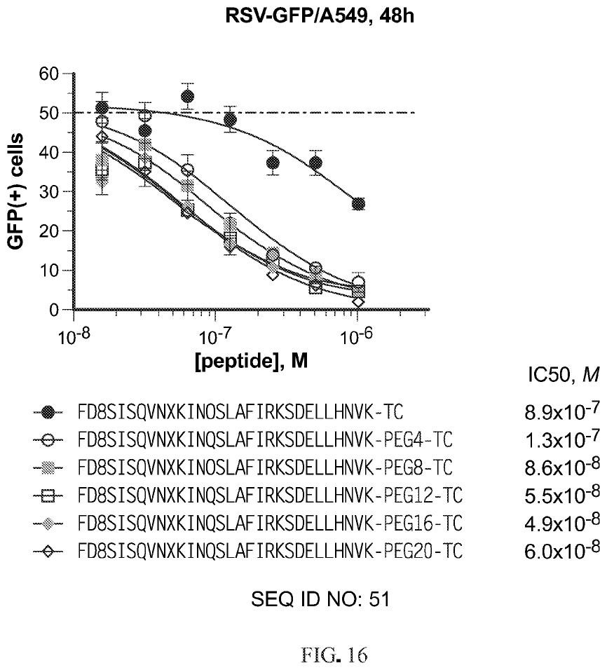

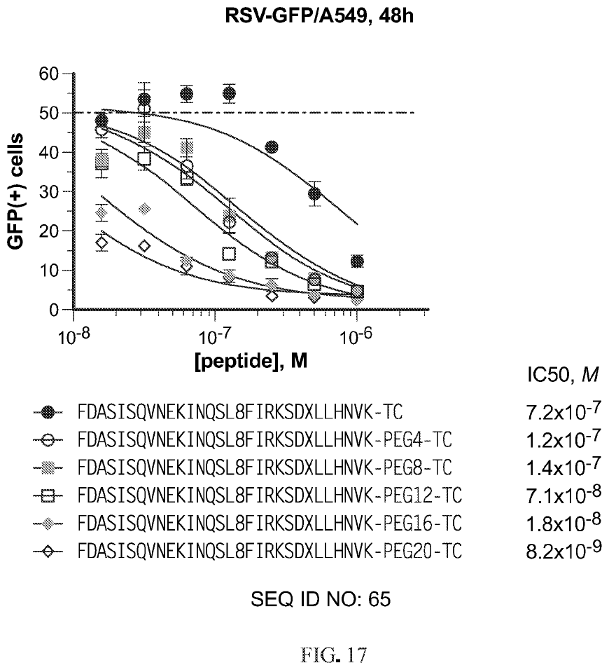

To determine the optimal PEG-chain length to link the stapled RSV HR2 peptide to the thiocholesterol moiety, a series of PEG(n) analogs were generated according to the above-described synthetic method, where n equals 0, 4, 8, 12, 16, or 20. The antiviral activity of this series of i, i+7 stapled RSV HR2 peptides bearing SEQ ID NO: 51 and 65 and thiocholesterol spaced by variable length PEG-linkers was examined in the GFP-RSV live virus assay (0.75-1.25 μL of virus per well; cells: A549; peptide doses of 2 μM; read-out at 48 hours). See FIG. 16 and FIG. 17. The superiority of the PEG16 and PEG20 linkers in particular, as compared to PEG4, PEG8, and PEG12, for example, was unexpected, requiring experimental determination in RSV antiviral assays. The PEG16-thiochol and PEG20-thiochol C-terminal derivatizations of SEQ IQ NO: 5 produced compounds with IC50s of 49 and 60 nM, respectively (see, FIG. 16), reflecting a greater than two orders of magnitude improvement in anti-viral activity compared to an RSV HR2 peptide bearing the corresponding single staple alone and with an additional 6 HR2 amino acids at the C-terminus (see, FIG. 4). Notably, the PEG16-thiochol and PEG20-thichol C-terminal derivations of an alternatively single-stapled peptide of SEQ ID NO: 21 produced compounds with IC50s of 18 and 8.2 nM (see, FIG. 17). Thus, the combination of staple scanning, differential peptide truncation, and PEG scanning revealed a staple location, sequence length, and PEG chain length that produced a stapled lipopeptide of SEQ ID NO:21 with a strikingly high increase in anti-RSV activity (a single digit nanomolar anti-RSV activity), reflecting a three orders of magnitude improvement over the singly stapled RSV HR2 peptide of SEQ ID NO: 4 and corresponding superiority to previously reported double-stapled anti-RSV peptides of longer length and lacking the PEG-thiocholesterol derivatization (Bird et al. JCI, 2014, 124(5):2113-2124).

### Example 4: Rescuing Poor Solubility by Appending Three Native N-Terminal Residues

Poor solubility was observed for the stapled lipopeptide with SEQ ID NO:21 but installing three native N-terminal residues “SDE,” which included two negatively charged amino acids, fully resolved the insolubility challenge, resulting in complete solubility at 15 mg/mL in 15% DMSO, PBS pH 7.4. The sequence optimization shifted the pI from 8, which corresponds to poor solubility at neutral pH, to a pI of 4.7 with complete solubilization.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patents, patent applications, internet sites, and accession numbers/database sequences including both polynucleotide and polypeptide sequences cited herein are hereby incorporated by reference in their entirety for all purposes to the same extent as if each individual publication, patent, patent application, internet site, or accession number/database sequence were specifically and individually indicated to be so incorporated by reference.

What is claimed is:

1. A conjugate comprising (i) a structurally-stabilized peptide and (ii) cholesterol or thiocholesterol;
wherein the cholesterol or thiocholesterol are linked, directly or via a linker, to the C-terminal amino acid of the structurally-stabilized peptide;
wherein the structurally-stabilized peptide comprises an internally cross-linked amino acid sequence comprising (i) 25 to 29 contiguous amino acids of the sequence FDASISQVNEKINQSLAFIRKSDELLHNV (SEQ ID NO:100), or (ii) the sequence SDEFDASISQVNEKINQSLAFIRKSDELLHNV (SEQ ID NO:280), except for two to six amino acid substitutions relative to the sequence of SEQ ID NO:100 or 281 and wherein the internally cross-linked amino acid sequence does not include the sequence NAGKST (SEQ ID NO:258);
wherein two of the two to six amino acid substitutions are with α, α-disubstituted non-natural amino acids with olefinic side chains cross-linked to each other, wherein the α, α-disubstituted non-natural amino acids with olefinic side chains cross-linked to each other are separated by three or six amino acids;
wherein the conjugate binds to an RSV 5-helix bundle protein and/or wherein the conjugate inhibits infection of a cell by RSV and/or prevents infection of a cell by RSV; and
wherein the conjugate is 25 to 45 amino acids in length, optionally wherein the conjugate is 30 amino acids in length.

2. A conjugate comprising (i) a structurally-stabilized peptide and (ii) cholesterol or thiocholesterol;
wherein the cholesterol or thiocholesterol are linked, directly or via a linker, to the C-terminal amino acid of the structurally-stabilized peptide;
wherein the structurally-stabilized peptide comprises an internally cross-linked amino acid sequence having the formula: 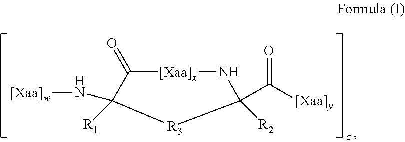
or a pharmaceutically acceptable salt thereof;
wherein each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted;
wherein x is 3 or 6;
wherein each R₃ is independently alkylene, alkenylene, or alkynylene, any of which is substituted or unsubstituted;
wherein z is 1, 2, 3, 4, 5, 6, 7, 8, 9, or 10;
wherein the internally cross-linked amino sequence comprises (i) 25 to 29 contiguous amino acids of the sequence of SEQ ID NO:100, or (ii) the sequence SDEFDASISQVNEKINQSLAFIRKSDELLHNV (SEQ ID NO:280), except for two to six amino acid substitutions relative to the sequence of SEQ ID NO:100 or 281 and wherein the internally cross-linked amino acid sequence does not include the sequence NAGKST (SEQ ID NO:258);
wherein the conjugate binds to an RSV 5-helix bundle protein and/or wherein the conjugate inhibits infection of a cell by RSV and/or prevents infection of a cell by RSV; and
wherein the conjugate is 25 to 45 amino acids in length, optionally wherein the conjugate is 30 amino acids in length.

3. The conjugate of claim 1 or 2, comprising the cholesterol, optionally wherein the linker comprises PEG.

4. The conjugate of claim 1 or 2, comprising the thiocholesterol, optionally wherein the linker comprises PEG.

5. The conjugate of claim 1 or 2, wherein the conjugate comprises PEG(n)-cholesterol directly linked to the C-terminal amino acid of the structurally-stabilized peptide, wherein n is 1-36, optionally wherein n is 4, 5, 6, 7, 8, 12, 16, or 20.

6. The conjugate of claim 1 or 2, wherein the conjugate comprises PEG(n)-thiocholesterol directly linked to the C-terminal amino acid of the structurally-stabilized peptide, wherein n is 1-36, optionally wherein n is 4, 5, 6, 7, 8, 12, 16, or 20.

7. The conjugate of claim 1 or 2, wherein the conjugate comprises Formula III directly linked to the C-terminal amino acid of the structurally-stabilized peptide: 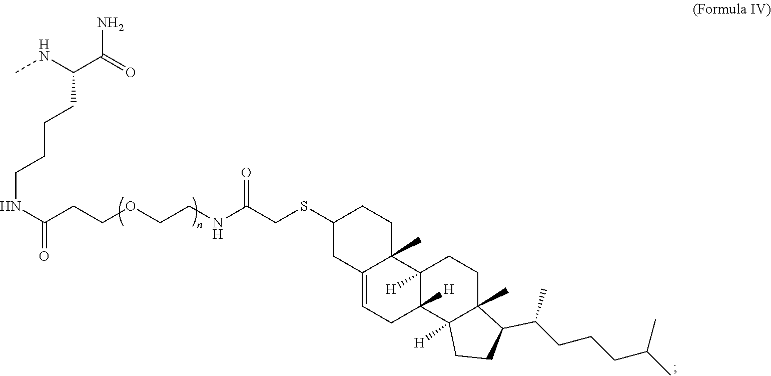
wherein n is 1-36, optionally wherein n is 4, 5, 6, 7, 8, 12, 16, or 20.

8. The conjugate of claim 1 or 2, wherein the conjugate comprises Formula II directly linked to the C-terminal amino acid of the structurally-stabilized peptide: 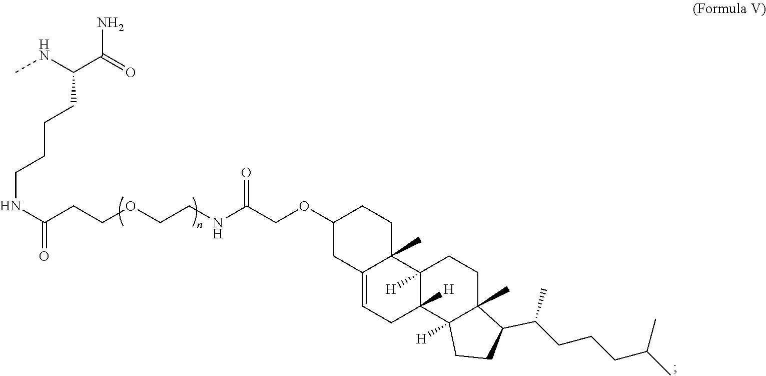
wherein n is 1-36, optionally wherein n is 4, 5, 6, 7, 8, 12, 16, or 20.

9. The conjugate of claim 1, wherein the α, α-disubstituted non-natural amino acids with olefinic side chains cross-linked to each other are separated by three amino acids, optionally wherein each of the α, α-disubstituted non-natural amino acids with olefinic side chains cross-linked to each other is (S)-α-(4′-pentenyl)alanine.

10. The conjugate of claim 1, wherein the α, α-disubstituted non-natural amino acids with olefinic side chains cross-linked to each other are separated by six amino acids, optionally wherein the α, α-disubstituted non-natural amino acids with olefinic side chains cross-linked to each other are (R)-α-(7′-octenyl)alanine and (S)-α-(4′-pentenyl)alanine.

11. The conjugate of claim 1, wherein the two substitutions with α, α-disubstituted non-natural amino acids with olefinic side chains cross-linked to each other are at the amino acids corresponding to positions 1 and 8 of the sequence set forth in SEQ ID NO:100, at the amino acids corresponding to positions 3 and 10 of the sequence set forth in SEQ ID NO:100, or at the amino acids corresponding to positions 17 and 24 of the sequence set forth in SEQ ID NO:100.

12. The conjugate of claim 2, wherein R₃ is an internal cross-link between the amino acids corresponding to positions 1 and 8 of the sequence set forth in SEQ ID NO:100, between the positions 3 and 10 of the sequence set forth in SEQ ID NO:100, or between the positions 17 and 24 of the sequence set forth in SEQ ID NO:100.

13. The conjugate of any one of claims 1 to 8, wherein the conjugate comprises the sequence set forth in any one of SEQ ID NOs: 5-26 and 71-92.

14. The conjugate of any one of claims 1 to 8, wherein the structurally-stabilized peptide comprises the sequence set forth in any one of SEQ ID NOs:27, 29, 36, 43, and 46.

15. The conjugate of any one of claims 1 to 8, wherein the structurally-stabilized peptide comprises the sequence set forth in any one of SEQ ID NOs:49, 51, 58, 65, 68, and 281.

16. The conjugate of claim 1 or 2, wherein the conjugate comprises the sequence set forth in any one of SEQ ID NOs:71, 73, 80, 87, and 90.

17. The conjugate of claim 1 or 2, wherein the conjugate comprises the sequence set forth in any one of SEQ ID NOs:5, 6, 14, 21, and 24.

18. The conjugate of any one of claims 1 to 17, wherein the conjugate is 25 to 34, 26 to 33, 27 to 32, 28 to 31, 29, or 30 amino acids in length.

19. A conjugate comprising or consisting of 8DASISQXNEKINQSLAFIRKSDELLHNV* (SEQ ID NO:265), wherein 8 is internally cross-linked to X, wherein 8 is (R)-α-(7′-octenyl)alanine, wherein X is (S)-α-(4′-pentenyl)alanine, wherein * is the formula 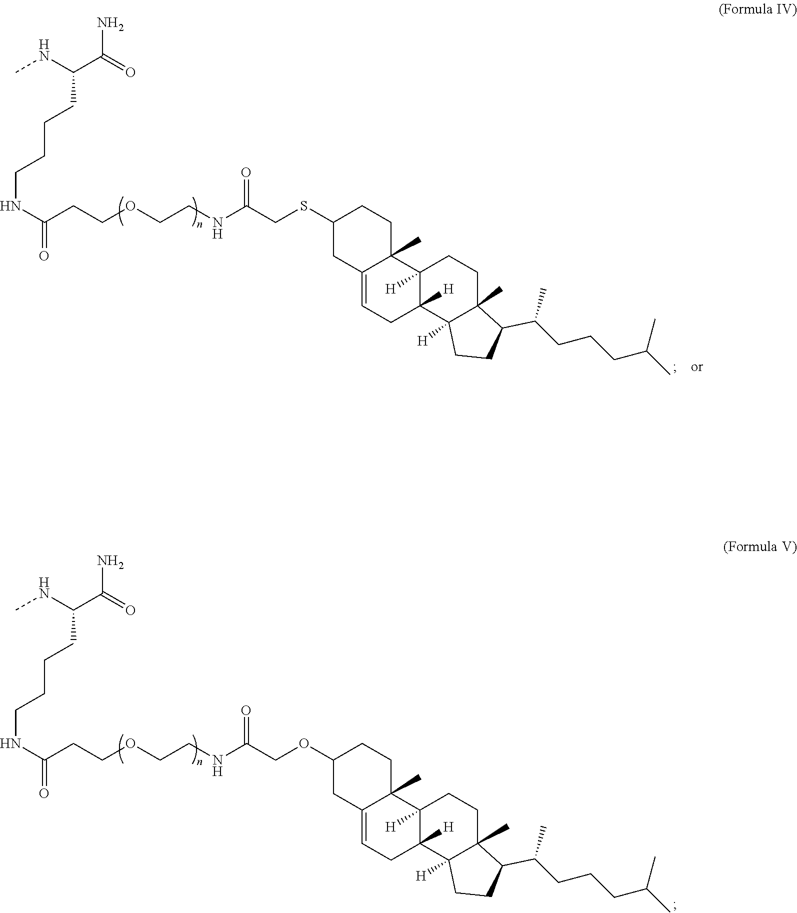
wherein n is 1-36, optionally wherein n is 16 or 20.

20. A conjugate comprising or consisting of FD8SISQVNXKINQSLAFIRKSDELLHNV * (SEQ ID NO:261), wherein 8 is internally cross-linked to X, wherein 8 is (R)-α-(7′-octenyl)alanine, wherein X is (S)-α-(4′-pentenyl)alanine, wherein * is the formula 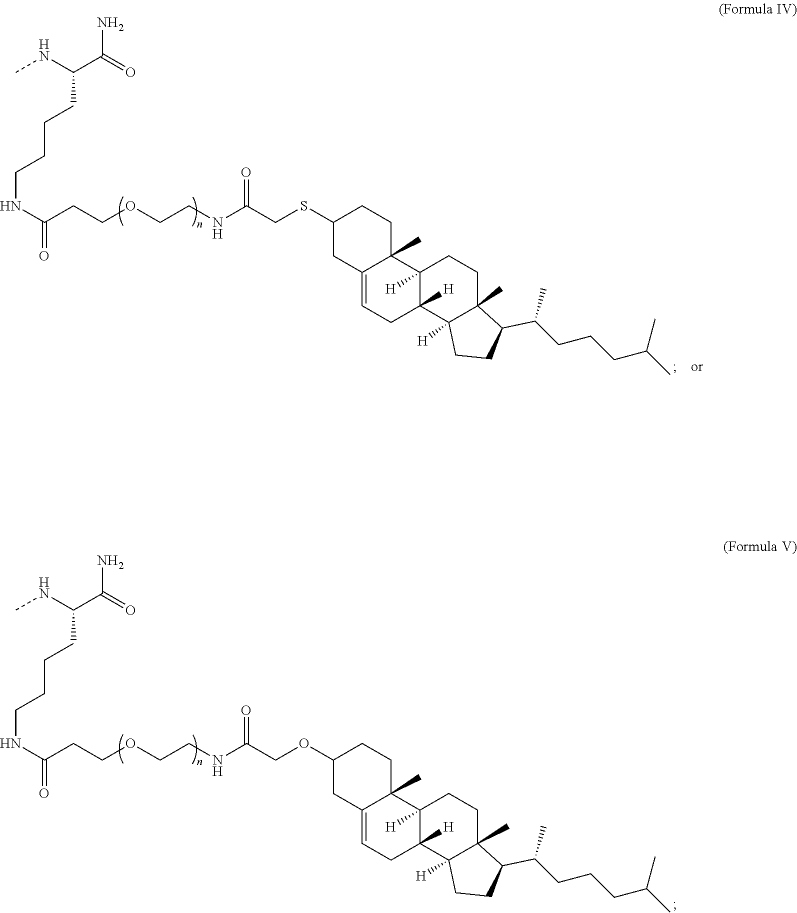
wherein n is 1-36, optionally wherein n is 16 or 20.

21. A conjugate comprising or consisting of FDASISQVNEKINQSL8FIRKSDXLLHNV* (SEQ ID NO:266), wherein 8 is internally cross-linked to X, wherein 8 is (R)-α-(7′-octenyl)alanine, wherein X is (S)-α-(4′-pentenyl)alanine, wherein * is the formula 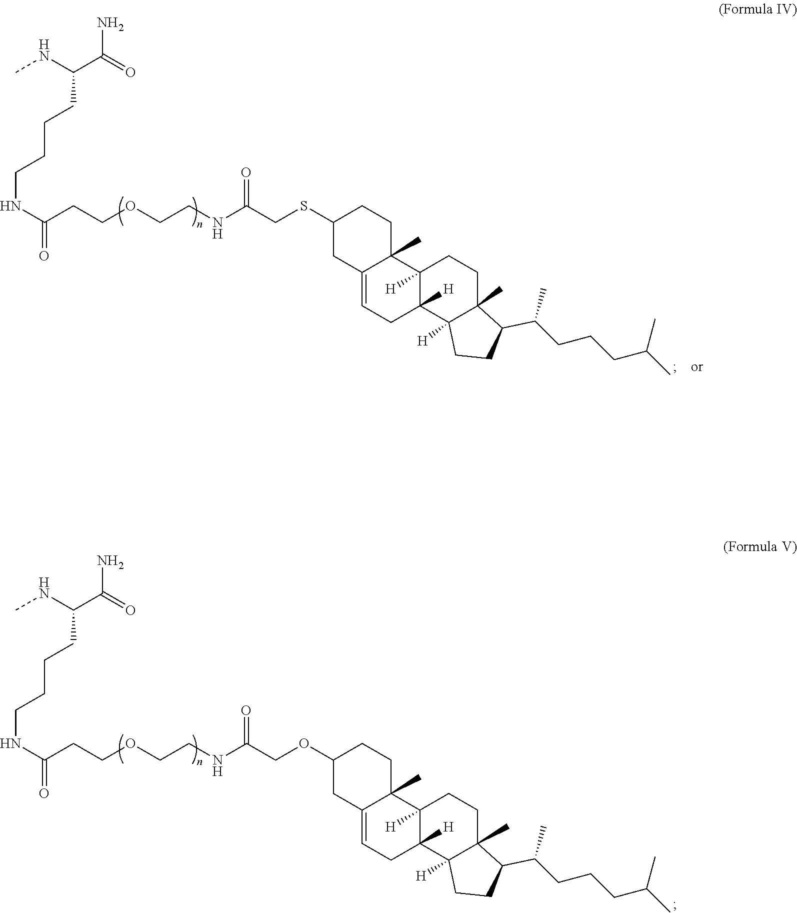
wherein n is 1-36, optionally wherein n is 16 or 20, optionally wherein the conjugate comprises three N-terminal amino acids X1X2X3 immediately upstream of the first F of SEQ ID NO:266, wherein X₁ is any amino acid, optionally S, and X2 and X3 are negatively charged amino acids.

22. A structurally-stabilized peptide, comprising:
an internally cross-linked amino acid sequence comprising 25 to 29 contiguous amino acids of the sequence FDASISQVNEKINQSLAFIRKSDELLHNV (SEQ ID NO:100), except for two to six amino acid substitutions relative to the sequence of SEQ ID NO:100;
wherein two of the two to six amino acid substitutions are with α, α-disubstituted non-natural amino acids with olefinic side chains cross-linked to each other, wherein the α, α-disubstituted non-natural amino acids with olefinic side chains cross-linked to each other are separated by three or six amino acids;
wherein the structurally-stabilized peptide binds to an RSV 5-helix bundle protein and/or wherein the structurally-stabilized peptide inhibits infection of a cell by RSV and/or prevents infection of a cell by RSV; and
wherein the structurally-stabilized peptide is 25 to 32 amino acids in length, optionally wherein the structurally-stabilized peptide is 32 amino acids in length, optionally, wherein the structurally-stabilized peptide comprises three N-terminal amino acids X1 X2X3 immediately upstream of the first F of SEQ ID NO: 100, wherein X1 is any amino acid, optionally S, and X2 and X3 are negatively charged amino acids.

23. A structurally-stabilized peptide, comprising:
an internally cross-linked amino acid sequence having the formula: 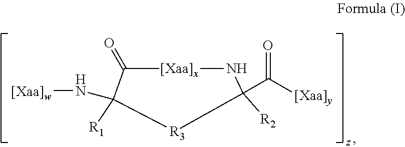
or a pharmaceutically acceptable salt thereof;
wherein each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted;
wherein x is 3 or 6;
wherein each R₃ is independently alkylene, alkenylene, or alkynylene, any of which is substituted or unsubstituted;
wherein z is 1, 2, 3, 4, 5, 6, 7, 8, 9, or 10; and
wherein the internally cross-linked amino sequence comprises 25 to 29 contiguous amino acids of the sequence of SEQ ID NO:100, except for two to six amino acid substitutions relative to the sequence of SEQ ID NO:100;
wherein the structurally-stabilized peptide binds to an RSV 5-helix bundle protein and/or wherein the structurally-stabilized peptide inhibits infection of a cell by RSV and/or prevents infection of a cell by RSV; and
wherein the structurally-stabilized peptide is 25 to 30 amino acids in length, optionally wherein the structurally-stabilized peptide is 29 amino acids in length.

24. The structurally-stabilized peptide of claim 22 or 23, which does not comprise the amino acids corresponding to positions 517-522, 517-521, 517-520, 517-519, or 517-518 of an RSV-F protein (numbered according to the sequence set forth in SEQ ID NO:1).

25. The structurally-stabilized peptide of claim 22 or 23, wherein the internally cross-linked amino acid sequence does not include the sequence NAGKST (SEQ ID NO:258).

26. The structurally-stabilized peptide of claim 22 or 23, wherein the structurally-stabilized peptide is 29 amino acids in length and comprises 29 contiguous amino acids of the sequence of SEQ ID NO:100, except for two to six amino acid substitutions relative to the sequence of SEQ ID NO:100.

27. The structurally-stabilized peptide of any one of claims 22 to 26, wherein the structurally-stabilized peptide comprises the amino acid sequence set forth in any one of SEQ ID NOs: 27-70.

28. The structurally-stabilized peptide of any one of claims 22 to 26, wherein the structurally-stabilized peptide comprises the amino acid sequence set forth in any one of SEQ ID NOs: 27, 29, 36, 43, and 46.

29. The structurally-stabilized peptide of any one of claims 22 to 26, wherein the structurally-stabilized peptide comprises the amino acid sequence set forth in any one of SEQ ID NOs: 49, 51, 58, 65, 281, 285, and 68.

30. A peptide, comprising the amino acid sequence of any one of SEQ ID NOs:27-70, except for zero to six additional substitutions, wherein the peptide does not include the sequence NAGKST (SEQ ID NO:258).

31. A pharmaceutical composition comprising the conjugate of any one of claims I to 21, the structurally-stabilized peptide of any one of claims 22 to 29, or the peptide of claim 30, and a pharmaceutically acceptable carrier.

32. A method of treating an RSV infection in a subject in need thereof, the method comprising administering to the subject a therapeutically-effective amount of the conjugate of any one of claims 1 to 21, the structurally-stabilized peptide of any one of claims 22 to 29, or the peptide of claim 30.

33. A method of preventing an RSV infection in a subject in need thereof, the method comprising administering to the subject a therapeutically-effective amount of the conjugate of any one of claims 1 to 21, the structurally-stabilized peptide of any one of claims 22 to 29, or the peptide of claim 30.

34. The method of claim 32 or 33, wherein the subject is a human.

35. A method of making a structurally-stabilized peptide, the method comprising:
I. (a) providing a peptide having an amino acid sequence comprising 25 to 29 contiguous amino acids of the sequence FDASISQVNEKINQSLAFIRKSDELLHNV (SEQ ID NO:100), except for two to six amino acid substitutions relative to the sequence of SEQ ID NO:100, ;
wherein two of the two to six amino acid substitutions are with α, α-disubstituted non-natural amino acids with olefinic side chains, wherein the α, α-disubstituted non-natural amino acids with olefinic side chains are separated by three or six amino acids; and
(b) cross-linking the peptide, thereby making the structurally-stabilized peptide, and optionally purifying the structurally-stabilized peptide; or
II. (a′) providing a peptide comprising the sequence SDEFDASISQVNEKINQSLAFIRKSDELLHNV (SEQ ID NO:280), except for two to six amino acid substitutions relative to the sequence of SEQ ID NO:280;
wherein two of the two to six amino acid substitutions are with α, α-disubstituted non-natural amino acids with olefinic side chains, wherein the α, α-disubstituted non-natural amino acids with olefinic side chains are separated by three or six amino acids; and
(b′) cross-linking the peptide, thereby making the structurally-stabilized peptide, and optionally purifying the structurally-stabilized peptide.

36. The method of claim 35, wherein the cross-linking is by a ruthenium catalyzed metathesis reaction.

37. The method of claim 35 or 36, further comprising derivatizing a resin bound amine of the structurally-stabilized peptide with PEG and/or cholesterol or thiocholesterol containing a carboxylic acid on a resin.

38. The method of any one of claims 35 to 37, further comprising formulating the structurally-stabilized peptide as a sterile pharmaceutical composition.
